(19) United States
(12) Patent Application Publication
(10) Pub. No.: US 2003/0129262 A1
(43) Pub. Date: Jul. 10, 2003

(54) Methionine restriction for cancer therapy

(76) Inventors: Daniel E. Epner, Houston, TX (US); Jeffrey H. Baxter, Galena, OH (US); Jeffrey T. Kivi, Westerville, OH (US); Robert G. Hards, Delaware, OH (US)

Correspondence Address: FULBRIGHT & JAWORSKI, LLP, 1301 MCKINNEY, SUITE 5100, HOUSTON, TX, 77010-3095, US

(21) Appl. No.: 10/231,562

(22) Filed: Aug. 30, 2002

Related U.S. Application Data
(60) Provisional application No. 60/316,142, filed on Aug. 30, 2001.

(51) Int. Cl. A61K 38/48; A61K 35/78

(52) U.S. Cl. 424/757; 424/94.63

(57) ABSTRACT

The present invention is directed to methods and compositions regarding induction of apoptosis in a cell and treatment of cancer in an individual wherein methionine deprivation of the cell is followed by methionine repletion of the cell. The methionine deprivation of the cell results in G2 arrest in the cell, leading to DNA damage in the cell. Upon readministration of methionine to the cell, the damaged cell exits G2 arrest and enters into apoptosis. In a specific embodiment, a therapeutic effect is elicited by cycling the patient between a methionine-depleted diet and a normal, methionine-replete diet. The present invention also regards chronic methionine deprivation for treatment of cancer in a human, in addition to methods for methionine restriction as adjunctive therapy for other cancer treatment methods.

This application claims priority to U.S. Provisional Patent Application Serial No. 60/316,142, filed Aug. 30, 2001, incorporated by reference herein in its entirety.

The United States Government may own certain rights in the invention pursuant to funding from the U.S. Department of Veterans Affairs.

### FIELD OF THE INVENTION

The present invention is directed to the fields of cancer biology and nutrition. Specifically, the present invention regards induction of apoptosis in a cancer cell. More specifically, the present invention is directed to use of methionine level-reducing agents, such as methionine-restricted diets, for cancer treatment.

### BACKGROUND OF THE INVENTION

Hundreds of thousands of individuals die each year of the most common malignancies-such as those originating in the prostate, breast, lung, and gastrointestinal tract. Surgery and radiation treatment save many lives, but far too many people develop metastatic cancer, which is often resistant to chemotherapy and therefore lethal. Smoking cessation, consumption of diets rich in fruits and vegetables, avoidance of excessive sun exposure, and other lifestyle measures can prevent many cancers (“Food, Nutrition and the Prevention of Cancer: A Global Perspective,” American Institute for Cancer Research, 1997). Unfortunately, implementation of these conceptually simple measures remains elusive.

Chemotherapy cures only a few types of metastatic cancer, including certain hematological malignancies, germ cell tumors, and a small fraction of other tumors. Unfortunately, the vast majority of common metastatic cancers, including those originating in the breast, prostate, gastrointestinal tract, and lung, are lethal. Therefore, novel treatment strategies for metastatic cancer are desperately needed. Fortunately, advances in molecular biology in recent years have led to the discovery of several promising targets for cancer treatment: dietary amino acid restriction is one such strategy.

Animal experiments published in the early 1900s focused on the potential antitumor activity of dietary protein or amino acid restriction (Drummond, 1917). These initial studies demonstrated no antitumor activity, leading Drummond to conclude “it is not possible to bring about an inhibition of tumor growth by an employment of dietary restrictions”. This pessimistic conclusion, however, was not justified in light of later, more sophisticated studies involving chemically defined diets. In 1959, Sugimara and colleagues studied the antitumor activity in tumor-bearing animals of diets lacking one essential amino acid. They found that tumor growth in animals was considerably slowed by diets lacking methionine, isoleucine, or valine. Of the three diets, the methionine free diet was the least toxic, which strongly indicated that dietary methionine restriction has highly specific effects on tumors and host tissues and does not represent indiscriminate tumor “starvation”.

Beginning in the early 1970's, many studies focused on the potential antitumor activity of methionine restriction. Mammalian cells cannot synthesize methionine from any of the other standard amino acids but can remethylate homocysteine to methionine (Hoffman and Erbe, 1976) (FIG. 1). Normal cells can therefore grow in culture when methionine is replaced by homocysteine in the growth medium (Stem and Hoffman, 1986), and animals fed diets in which methionine has been replaced by homocysteine suffer no ill effects and grow normally (Du Vigneaud et al., 1939; Breillout et al., 1987). In contrast, a wide variety of cancer cell lines are methionine dependent even in the presence of homocysteine.

Numerous cell culture studies using normal and malignant cell lines (e.g., leukemia, prostate) demonstrated that methionine restriction suppresses cancer cell growth, with little or no deleterious effect on normal cells (Kreis, 1979; Tisdale and Eridani, 1981; Mecham et al., 1983; Stem et al., 1984; Breillout et al., 1990; Poirson-Bichat et al., 1997). Likewise, tumors are methionine dependent in vivo. Dietary methionine restriction causes regression of a variety of animal tumors and inhibits metastasis in animal models (Breillout et al., 1987; Breillout et al., 1990; Guo et al., 1993; Goseki et al., 1987; Milllis et al., 1996; Millis et al., 1998). For example, altering the dietary arginine-methionine balance inhibited tumor growth without causing cachexia in rats with subcutaneously transplanted Morris hepatoma Millis et al., 1996; Millis et al., 1998). Also, substituting homocysteine for methionine reduced tumor growth and metastasis in a rat rhabdomyosarcoma cell line (Breillout et al., 1987; Breillout et al., 1990). In addition, many fresh patient tumors in primary histoculture and human tumor xenografts in nude mice are methionine dependent (Guo et al., 1993; Hoshiya et al., 1995).

Methioninase, an enzyme that specifically degrades methionine, inhibits growth of a variety of cancer cells in culture as well as solid tumors and leukemia in animals (Kreis et al., 1979; Tan et al., 1999; Yoshioka et al., 1998; Tan et al., 1996; Kreis and Hession, 1973; Tisdale et al., 1983; Yoshioka et al., 1998; Kokkinakis et al., 1997; Kreis et al., 1980; Miki et al., 2000). Animal studies also support an anti-tumor effect of methioninase (Tan et al., 1996; Kreis and Hession, 1973; Yoshioka et al., 1998). In laboratory animals with Lewis lung carcinoma, synergistic antitumor activity was demonstrated between methioninase and the chemotherapy drug 5-fluorouracil (Yoshioka et al., 1998). In an orthotopic lung cancer model, recombinant methioninase plus methioninase gene therapy was effective in suppressing cancer (Miki et al., 2000). The fact that methioninase inhibits tumor growth in preclinical models further supports the concept of dietary methionine restriction as cancer treatment. Recombinant methioninase was recently tested in a phase I clinical trial in Mexico. Patients in the trial experienced no significant toxicity, and plasma methionine levels fell dramatically as expected (Tan et al., 1996; Tan et al., 1997), although the antitumor activity of methioninase was not assessed in this trial. A polyethylene glycol conjugation of methioninase has been developed to reduce the potential antigenicity and lengthen the half-life of the recombinant enzyme (Tan et al., 1998a; Tan et al., 1998b).

Determining the molecular mechanisms for the tumor specific growth inhibitory effects of methionine restriction requires an understanding of the specialized functions of methionine. Methionine is the major methyl donor for methylation of DNA, RNA, proteins, and other molecules. Overall rates of methylation are much higher in tumors than in normal tissues (Stem and Hoffman, 1984; Tisdale, 1980; Judde et al., 1989). Cytosine methylation within CpG islands is one of the mechanisms by which gene expression is regulated (Baylin et al., 1998). Several growth inhibitory and pro-apoptotic genes are transcriptionally silenced in tumors as a result of focal DNA hypermethylation. DNA methylation also compacts and stabilizes chromatin structure and decreases its susceptibility to DNA-damaging agents. Loss of methylated cytosines reduces the stability of chromatin by decreasing binding sites for methyl-specific DNA-binding proteins (Nan et al., 1997). In the absence of methyl-directed protein binding, affected DNA sequences are rendered more accessible to oxidant and/or enzyme-induced DNA strand breakage (Pogribny et al., 1995; Davey et al., 1997; Razin, 1998; Wolffe, 1998). Animal studies have demonstrated that severe, prolonged methyl deficiency induced by dietary restriction of methionine, choline, homocysteine, and folate leads to global demethylation of normal liver DNA and resultant increased susceptibility to DNA strand breaks (Pogribny et al., 1995). Inhibition of DNA methylation by methionine restriction may therefore make cancer cell DNA susceptible to damage.

Methionine is also required for synthesis of polyamines, which have far-ranging effects on nuclear structure and cell division (“Polyamines in Cancer: Basic Mechanisms and Clinical Approaches, 1996), and for glutathione homeostasis. Glutathione (γ-glutamylcysteinylglycine) is a ubiquitous tripeptide that reduces oxidative stress in cells. Oxidative stress is primarily due to reactive oxygen species generated from mitochondrial respiration that are known to damage nuclear and mitochondrial DNA, as well as many other molecules (Beckman and Ames, 1997; Davies, 1995). Certain toxins and drugs, such as cancer chemotherapy drugs, also cause oxidative stress. Many tumors contain elevated levels of glutathione that confer resistance to a variety of chemotherapy drugs (Chen et al., 1998; Calvert et al., 1998). Methionine maintains intracellular glutathione levels by acting as a sulfur donor for synthesis of cysteine in the liver and by preventing efflux of glutathione from within cells (Fernandez-Checa et al., 1990; Aw et al., 1984). Therefore, methionine restriction potentially could inhibit tumor growth by inducing oxidative DNA damage in cancer cells.

Furthermore, that methionine restriction can potentially function as a chemotherapy adjuvant by sensitizing tumor cells to the cytotoxic effects of various chemotherapy agents is also known. As examples, a synergistic effect between methionine-deprivation and cisplatin (Hoshiya et al., 1996), nimustine (Goseki and Endo, 1990), and 5-fluorouracil (Yoshioka et al., 1998) have all been demonstrated in mice models. There are many putative molecular mechanisms by which this synergy is mediated. Lowered intratumoral levels of glutathione is one likely mechanism. Decreased global methylation status of DNA and resultant decreased compactness/stability of chromatin structure is another mechanism. A looser chromatin structure would cause DNA to be more susceptible to damage by chemotherapy agents. Furthermore, Lu et al. (2002) demonstrated that overexpression of c-jun N-terminal kinase 1 (JNK1)-enhanced apoptosis in response to methionine restriction, and they concluded that JNK1 plays a critical role in signaling cancer cells to undergo apoptosis in response to methionine restriction.

The absence of exogenous sources of methionine is known to arrest tumor cells in the G2 phase of the cell cycle. This effect has been demonstrated both in vitro and, more recently, in vivo (rats) and often reported in the scientific literature. This effect has been used in animal studies (Nagahama et al., 1998) to induce cell-cycle arrest for the purpose of cell-cycle alignment. Once aligned, these tumor cells are again fed methionine to allow them to escape cell-cycle arrest. At the same time, a cell-cycle specific chemotherapeutic agent is introduced. Its efficiency is enhanced due to the tumor cells predominantly being aligned in the same phase of the cell cycle and matched to an appropriate chemotherapeutic agent that exerts its effects most potently during a particular cell-cycle phase.

In the above example, the purpose of the dietary cycling is solely for the purpose of cell-cycle alignment as a means of increasing the effectiveness of a cell-cycle specific chemotherapeutic agent. This stands in contrast to the present invention, which is premised on the concept that cycling with a methionine level-reducing agent, particularly a plasma-methionine level-reducing agent and a methionine replete diet, alone, is sufficient to exert a therapeutic effect by first arresting tumor cells and then pushing those tumor cells down the pathway of apoptosis.

U.S. Pat. No. 5,817,695 is directed to a nutritional product for cancer patients with a below normal concentration of L-phenylalanine, L-tyrosine, and L-methionine and L-leucine is present in excess. The product is also used as an adjunct to conventional cancer therapies.

U.S. Pat. No. 5,571,510 regards selective methionine starvation of tumor cells which are methylthioadenosine phosphorylase negative. The cells are treated with methioninase, and in some embodiments, the methioninase is conjugated to polyethylene glycol. In some embodiments, the methioninase is administered via regional chemotherapeutic administrations, and in some embodiments, the methioninase is administered parenterally, preferably by intra-arterial infusion.

U.S. Pat. No. 5,658,895 is directed to an anticancer enteral feeding composition lacking the sulfur-containing amino acids methionine and cyst(e)ine. The composition comprises a powder obtained by emulsifying a fat in an aqueous solution of amino acids and spray-drying the resultant emulsion. Subsequently, granulated dextrin is dry-blended into the final product.

U.S. Pat. No. 5,208,039 addresses a nutrient composition deprived of methionine and supplemented with homocysteine for treatment of tumors. In some embodiments the composition is administered enterally, whereas in other embodiments the composition is administered parenterally. The patent teaches that “of course, the treatment . . . will generally be an additional treatment associated with other methods of therapy such as chemotherapy or radiotherapy.”

U.S. Pat. No. 6,017,962 is directed to administering to an individual for cancer a pharmacologically effective amount of a methionine scavenger, such as methioninase, and treating the individual, preferably intravenously or intraperitoneally, with homocystine while excluding dietary methionine, homocysteine and choline. U.S. Pat. No. 5,690,929 regards administration of methioninase in combination with chemotherapeutic agents to increase the therapeutic effectiveness of the agent.

Although the prior art is directed to methods and compositions for reducing methionine levels in a tumor cell for its treatment, none regard reducing the levels with an agent, such as with a diet, and administering a methionine-rich diet in succession (i.e. a cycled regimen); in specific embodiments, the alternating regimen is repeated. Additionally, where a chronic regimen is involved, other references are directed to dietary methods/compositions which are much more complex and/or severe than the chronic regimen disclosed herein. Where dietary strategies are involved, other references teach depletion of various dietary components such as choline, cyst(e)ine, cobalamine, and/or folate (in addition to depletion of methionine). Many known references specify the replacement of methionine by the addition of homocysteine to the diet, whereas others involve the intravenous administration of the enzyme methioninase. All of these additional complexities are the result of extrapolating results from animal studies (i.e. mice) to expectations for human studies. However, it is much more difficult to reduce plasma methionine levels in mice than it is in humans, and this is amply demonstrated by clinical results reported herein. Thus, known methods and compositions teach away from simple methionine restriction as being adequate to achieve therapeutic reductions in circulating plasma methionine levels.

The simplified dietary regimen/compositions disclosed herein are vastly preferable to known compositions. For example, depletion of the diet of choline, cyst(e)ine, cobalamin, and/or folate is more likely to produce deleterious side-effects than depletion of methionine alone. Also, homocysteine is a known strong risk factor for the induction of atherosclerosis. Finally, intravenous administration of the enzyme methioninase poses the risk of an antigenic response. Thus, the present invention is directed to overcome these deficiencies and disadvantages of known methods and compositions regarding methionine restriction and cancer.

### SUMMARY OF THE INVENTION

In an object of the present invention there are methods and compositions directed to induction of apoptosis in a cell and treatment of cancer in an individual wherein methionine deprivation of the cell is followed by methionine repletion of the cell.

In an object of the present invention, there is a cycled dietary regimen for accelerated induction of apoptosis in tumor cells. The cycled dietary regimen comprises, in some embodiments, delivery of a diet substantially lacking in methionine and/or a methionine-restricted diet to a cancer-stricken mammal, such as a human, followed by delivery of a methionine replete diet.

In another object of the present invention, there is a chronic diet for tumor growth inhibition and extension of life, particularly wherein the diet consists essentially of a methionine-reduced composition. In a specific embodiment of the chronic diet, methionine is the sole amino acid present at reduced levels in a balanced elemental amino acid composition. In a specific embodiment, methionine is present at reduced levels in a naturally occurring dietary composition incorporating intact protein(s).

In an additional object of the present invention, there is adjuvant therapy with the diet of the present invention to improve the efficacy of a specific chemotherapeutic agent for a particular cancer type.

In another object of the present invention, there are formulations of a methionine level-reduced diet with improved organoleptic characteristics to increase patient acceptance and compliance for cancer therapy. In a specific embodiment, there is an elemental diet having flavoring/masking agents to improve organoleptic characteristics. In another specific embodiment, there is a specialized protein for a diet having zero or low levels of methionine. This intact protein provides for drastically improved organoleptic properties when compared to an elemental diet.

Thus, in accordance with the foregoing objects of the present invention, the following summary of the present invention will highlight the salient issues involved.

Most metastatic tumors, such as those originating in the prostate, lung, and gastrointestinal tract, respond poorly to conventional chemotherapy. Novel treatment strategies for advanced cancer are therefore desperately needed. Dietary restriction of the essential amino acid methionine offers promise as such a strategy, either alone or in combination with chemotherapy or other treatments. Numerous in vitro and animal studies demonstrate the effectiveness of dietary methionine restriction in inhibiting growth and eventually causing death of cancer cells. In contrast, normal host tissues are relatively resistant to methionine restriction.

In some embodiments, the present invention relies on dietary deprivation of methionine alone as a means for suppressing plasma methionine levels. More severe and/or complicated means for lowering plasma methionine levels are known in the art. For example, some describe the deprivation of multiple components from the diet (e.g. methionine, cyst(e)ine, choline, folate, cobalamin, etc.). Others describe the intravenous injection of the enzyme, methioninase, to lower plasma methionine levels. These efforts apparently stem from the results of animal studies which indicated that plasma methionine levels are very difficult to suppress to therapeutic levels (at least in mice/rats). However, the Examples provided herein indicate that dietary restriction of methionine, alone, is sufficient to lower plasma methionine to therapeutic levels (below approximately 10 micromolar in concentration, in particular for humans; analogous levels for other animals determined by known means in the art are within the scope of the present invention). The results from analysis of clinical plasma samples for adequately lowered methionine levels are supported by the evidence of positive-objective clinical responses in those same patients.

Other references describe supplementing the diet with homocyst(e)ine. This is done because completely methionine-free diets are eventually lethal to rats/mice. Homocyst(e)ine can act as a metabolic precursor for production of methionine by normal tissues (but typically not tumor cells) thus protecting the animal against the deleterious effects of methionine deprivation. This precaution appears unnecessary in humans, however, as demonstrated by the chronic (up to 10 months) administration of severely methionine-restricted diets in a clinical study described herein without undue adverse effects on the patients. In practice, dietary homocysteine would possibly reduce the effectiveness of the overall strategy, because much of the dietary homocysteine could be converted to methionine by the first pass through the liver. The newly converted methionine would be returned to circulation and then become available to the tumor.

Other references in the art employ different strategies and/or mechanisms of action than the present invention. Some references describe the chronic use of methionine depletion for tumor growth inhibition, whereas others describe methionine depletion as an adjuvant for sensitizing tumors to standard chemotherapies. Yet other references invoke cell-cycle arrest and subsequent cell-cycle alignment as a means of improving the efficiency of cell-cycle specific chemotherapeutic agents. However, none of these references describe the cycling between a methionine-depleted and a replete diet for the purposes of temporary cell-cycle arrest (and cumulative cell damage) and then induction of widespread apoptosis by forcing the tumor cells back into the cell-cycle progression.

The present invention is less draconian than other strategies, in that only methionine is depleted from the diet. Some references specify the depletion of other components of the diet (e.g., cyst(e)ine, choline, folate, cobalamin) in addition to methionine. These additional deletions would increase the probability of deleterious effects on the patient. Other references specify the intravenous injection/infusion of the enzyme methioninase, although repeated injections would risk eliciting an antigenic response. Some references specify replacing methionine with homocysteine to protect healthy tissue from starvation, although the inventors have found this to be unnecessary. This is significant since homocysteine is a known, strong risk factor for arteriosclerosis.

Many references specify only chronic administration of a modified diet (including methionine-deficiency) for tumor growth inhibition. Sometimes, in this mode, slow regression of the tumor has been seen. Presumably this slow effect is due to a small percentage flux of the tumor cells escaping cell-cycle arrest and proceeding with apoptosis. The cycling strategy of the present invention is likely to drastically compress the timeline for arrest and regression of tumor. This is because repletion of diet will cause a substantially large percentage of the tumor cells to re-enter the cell-cycle progression. Having already accumulated a significant amount of damage to their cellular machinery during the arrested cycle, widespread apoptosis of tumor cells will follow.

Thus, the present invention is directed to dietary methionine restriction for individuals with cancer, particularly advanced cancer. The Examples presented herein indicate that dietary methionine restriction is safe and feasible for the treatment of patients with advanced cancer. In addition, the Examples provide evidence of antitumor activity. That is, one patient with hormone-independent prostate cancer experienced a 25% reduction in serum prostate-specific antigen (PSA) after 12 weeks on the diet, and a second patient with renal cell cancer experienced an objective radiographic response. In a specific embodiment, the methionine restriction acts synergistically with other cancer treatments, such as chemotherapy, radiation, surgery, or gene therapy.

In a specific embodiment, the methods of the present invention are directed to treating, at least in part, a methionine-dependent cancer in an individual, such as glioblastomas, medulloblastomas, pancreatic adenocarcinomas, lung carcinomas and melanomas. Moreover, an advantage of the present invention is that it is known in the art that methionine deprivation is more universally applicable to a wide array of target cancer populations.

In some embodiments, patients with advanced solid tumors are administered dietary methionine restriction. In a specific embodiment, patients are maintained on an enteral diet, since parenteral nutrition is potentially toxic, expensive, and logistically difficult.

In an embodiment of the present invention, there is a method of inducing apoptosis in a mammalian cell, comprising the steps of inducing cell-cycle arrest by methionine depletion in the cell; and abrogating the arrest by methionine repletion in the cell. In a specific embodiment, the inducing and abrogating steps are repeated at least once.

In another embodiment of the present invention, there is a method of treating cancer in a mammal, comprising the steps of a) delivering to the mammal at least one methionine level-reducing agent for a sufficient time to induce cell cycle arrest in a cancer cell of the mammal; and b) replenishing the methionine, wherein the replenishment results in induction of apoptosis in the cancer cell. In a specific embodiment, the delivering and replenishing steps are repeated at least once. In another specific embodiment, the methionine level-reducing agent is a methionine-restricted diet. In an additional specific embodiment, the methionine-restricted diet comprises methionine levels no greater than about 2 mg/kg/day. In another specific embodiment, the methionine restricted diet comprises an intact protein having less than about 0.2% (w/w) methionine. In an additional specific embodiment, the intact protein is from a legume. In an additional specific embodiment, the methionine restricted diet comprises a processed whole food from which purification of a specific protein or proteins is not required. In a further specific embodiment, the legume is broad bean or garden pea. In another specific embodiment, the methionine level-reducing agent is an enzyme. In a further specific embodiment, the enzyme is methioninase.

In another embodiment, the delivery of the methionine level-reducing agent further comprises the steps of a) administering to the mammal a diet substantially lacking in methionine for a time t₁; and b) administering to the mammal a methionine-restricted diet for a time t₂. In a specific embodiment, the time t₁ is at least approximately 1 week. In another specific embodiment, the time t₂ is at least approximately 1 week. In a specific embodiment, the combination of the time t₁ and the time for t₂ is between about 3 weeks and about 15 weeks. In a further specific embodiment, the replenishing step further comprises administering to the mammal a methionine-replete diet for a time t₃. In an additional specific embodiment, the time t₃ is about 1 week.

In another embodiment of the present invention, there is a method of treating cancer in a mammal, comprising the steps of a) administering to the mammal a diet substantially lacking in methionine for a time t₁; b) administering to the mammal a methionine-restricted diet for a time t₂; and c) administering to the mammal a diet replete with methionine for a time t₃. In a specific embodiment, the methionine-restricted diet comprises methionine levels no greater than about 2 mg/kg/day. In another specific embodiment, the time t₁ is at least approximately 1 week. In a further specific embodiment, the time t₂ is at least approximately 1 week. In an additional specific embodiment, the combination of the time t₁ and the time t₂ is between about 3 weeks and about 15 weeks. In another specific embodiment, the time t₃ is about 1 week. In a further specific embodiment, at least one step in the method comprises enteral administration of the diet to the individual. In a specific embodiment, the method is repeated at least once. In another specific embodiment, the cancer is an advanced cancer. In a specific embodiment, the method further comprises the step of treating the cancer with chemotherapy, surgery, radiation, gene therapy, immunotherapy, biological therapy, differentiating agents, chemopreventive agents, or a combination thereof.

In specific embodiments of the present invention, the cancer is prostate, lung, breast, colon, glioma, gastric, skin, esophagus, squamous cell carcinoma of head and neck region, pancreas, small intestine, bladder and urinary collecting system, kidney, testes, ovary, rectum, anus, liver, brain, soft tissue or osteogenic sarcoma, Hodgkin's lymphoma, non-Hodgkin's lymphoma, leukemia, or melanoma.

In another embodiment of the present invention, there is a method of treating cancer in a mammal, consisting essentially of delivering to the mammal at least one methionine level-reducing agent. In a specific embodiment, the methionine level-reducing agent is a diet substantially lacking methionine or having reduced levels of methionine.

In an additional embodiment of the present invention, there is a method for improving efficacy of a cancer therapy for a mammal, comprising the steps of delivering to the mammal at least one methionine level-reducing agent for a time t₁; and administering the cancer therapy. In a specific embodiment, the method is repeated at least once. In another specific embodiment, the cancer is an advanced cancer. In a specific embodiment, the method further comprises the step of treating the cancer with chemotherapy, surgery, radiation, gene therapy, immunotherapy, biological therapy, differentiating agents, chemopreventive agents, or a combination thereof.

In another specific embodiment, the delivery of the methionine level reducing agent is prior to the administration of the cancer therapy. In an alternative specific embodiment, the delivery of the methionine level reducing agent is during the administration of the cancer therapy. In an additional specific embodiment, the methionine level-reducing agent is administered following the administration of the cancer therapy. In a further specific embodiment, the methionine level-reducing agent is a methionine restricted diet. In a specific embodiment, the cancer therapy is chemotherapy. In another specific embodiment, the cancer therapy is radiation. In a further specific embodiment, the cancer therapy is surgery. In an additional specific embodiment, the cancer therapy is chemotherapy, radiation, surgery, or a combination thereof. In another specific embodiment, the methionine-restricted diet comprises methionine levels no greater than about 2 mg/kg/day. In a further specific embodiment, the time t₁ is at least approximately 1 week.

In an embodiment of the present invention, there is a nutritional composition comprising an isolated intact protein having less than about 0.2% (w/w) methionine. In a specific embodiment, the nutritional composition for an individual with cancer, comprising an isolated intact protein having less than about 0.2% (w/w) methionine. In a specific embodiment, the intact protein is from a legume. In a further specific embodiment, the legume is garden pea or broad bean. In an additional specific embodiment, the intact proteins are obtained from a whole food which does not require purification of a specific protein or proteins.

In an additional embodiment of the present invention, there is a nutritional composition for an individual with cancer, wherein said composition comprises a protein system comprising from zero to about 0.3% (w/w) methionine; and no more than about 1.5% (w/w) cyst(e)ine. In a specific embodiment, the composition provides about 15-30% of calories from protein. In another specific embodiment, the composition substantially lacks homocysteine. In an additional specific embodiment, the composition further comprises choline, cobalamine, and folate.

In another embodiment of the present invention, there is a method of treating cancer in a human, consisting essentially of administering to the human a methionine-restricted diet. In a specific embodiment, the method further comprises administering to said human a cancer therapy.

In an additional embodiment of the present invention, there is a method of treating cancer in a human, consisting essentially of administering to the human a diet substantially lacking methionine. In a specific embodiment, the method further comprises administering to said human a cancer therapy.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 depicts an overview of methionine metabolism.

FIG. 2 is a schematic isolation procedure for the protein fractions of garden pea and broad bean (Malley et al., 1975).

FIG. 3 is SDS-PAGE analysis of yellow (Propulse), snow (Renee's Garden) and green (Kroger split) pea seeds.

FIG. 4 is SDS-PAGE analysis of Renee's garden snow pea seed extract.

FIG. 5 shows native PAGE separation of prepared albumin, legumin and vicilin fractions. Separation was achieved using 8 to 25% gradient Phast gels with a total running time of 280 volt-hours. Molecular weight standards are noted in kilodaltons. Lane 1=albumin, Lane 2=legumin, Lane 3=vicilin, Lane 4=molecular weight standards. Note: Legumin fraction contains some vicilin.

FIG. 6 depicts size exclusion chromatographic separation of legumins and vicilins. Unfractionated extract was run on an Altex 3000SW Spherogel-TSK column coupled to an Altex 4000SW Spherogel-TSK column. Buffer used was 50 mM sodium phosphate, 0.3 M sodium chloride, pH 7.2 at a flow rate of 1 ml/minute. Protein elution was monitored at 214 nm. Protein standards of known molecular weight were used to calibrate the system. Peak “A” denotes legumin and peak “B” denoted vicilin.

FIG. 7 is a schematic of vicilin purification using centrifugation. For isolation from pea seeds vicilins include vicilins and convicilins.

FIGS. 8A through 8E show nutrient intake for patients with metastatic cancer on a phase I clinical trial of dietary methionine restriction. Bars represent standard error. FIG. 8A is daily energy intake. FIG. 8B is daily total protein intake. FIG. 8C indicates daily energy intake for patients 1-4 versus patients 5-8. FIG. 8D shows daily total protein intake for patients 1-4 versus patients 5-8. Bars are smaller than corresponding symbols for weeks two and four (patients 1-4) and are therefore not visible. Week 12 (patients 1-4) represents a single patient. FIG. 8E shows daily methionine intake. The dashed lines in FIGS. 8B and 8D represent RDA for total protein intake (0.8 g/kg/day).

FIGS. 9A through 9C depict plasma amino acid levels for patients with metastatic cancer on a phase I clinical trial of dietary methionine restriction. FIG. 9A shows plasma methionine levels during the first two weeks. FIG. 9B shows plasma methionine levels for the first 20 weeks. FIG. 9C shows plasma homocysteine levels for the first 20 weeks. Bars represent standard error.

FIGS. 10A and 10B illustrate percent change in body mass index (BMI) for patients with metastatic cancer on a phase I clinical trial of dietary methionine restriction. FIG. 10A shows data for all patients. FIG. 10B shows patients 1-4 versus patients 5-8. Bars in 4A represent standard error. Bars were left off in 4B for clarity.

FIGS. 11A and 11B depict albumin and prealbumin levels. FIG. 11A shows serum albumin levels for patients with metastatic cancer on a phase I clinical trial of dietary methionine restriction. Bars represent standard error. FIG. 11B shows serum prealbumin levels for the seventh and eighth patients. The dashed line represents the lower limit of normal for serum prealbumin (18 mg/dL).

### DETAILED DESCRIPTION OF THE INVENTION

I. Definitions

The term “a” or “an” as used herein the specification may mean one or more. As used herein in the claim(s), when used in conjunction with the word “comprising”, the words “a” or “an” may mean one or more than one. As used herein “another” may mean at least a second or more.

The term “advanced cancer” as used herein is defined as cancer which is metastatic, is refractory to standard therapy, and/or for which no standard therapy exists.

The term “cachexia” as used herein is defined as a general lack of nutrition and/or wasting as the result of a chronic disease. In another embodiment, cachexia refers to weight loss as a result of the cannabilization of muscle tissue due to an inflammatory process mediated by cytokines from cancer cells.

A skilled artisan recognizes that the term “cyst(e)ine” as used herein refers to either of the two oxidation states for the molecule: cystine and cysteine.

The term “enteral” as used herein refers to administration through the alimentary tract. A skilled artisan recognizes that this administration may be within the intestine, which is the tube passing from the stomach to the anus divided into the i. tenue (small intestine) and i. crassum (large intestine), through the mouth, through a nasogastric tube into the stomach, and other means known in the art.

The term “intact protein” as used herein refers to a protein preferably not subjected to either chemical or enzymatic hydrolysis, and preferably is in a form substantially similar or identical to its natural state.

The term “methionine depleted diet” as used herein refers to two forms of a diet or nutritional composition: methionine-restricted diet and a diet substantially lacking in methionine.

The term “methionine level-reducing agent” refers to a diet or nutritional composition which reduces the methionine levels in the organism to which it is administered to.

The term “methionine replete diet” as used herein is defined as a diet or nutritional composition having typical levels of methionine for an individual with substantially normal dietary consumption. A skilled artisan recognizes that these levels of methionine in the diet meet Recommended Daily Intake (RDI) guidelines for caloric and methionine intake. In a specific embodiment, methionine levels are at least about 10 mg/kg/day. In another specific embodiment, levels are approximately 20-30 mg/kg/day of methionine. In an additional specific embodiment, the levels are approximately 22-25 mg/kg/day of methionine.

The term “methionine-restricted diet” as used herein is defined as a diet or nutritional composition comprising approximately 0-3 mg/kg/day of methionine. In specific embodiments, the diet comprises approximately 0.5, 1.0, 1.25, 1.5, 1.75, 1.8, 1.9, 2.0, 2.1, 2.2, 2.3, 2.4, 2.5, 2.6, 2.75, 2.8, or 3.0 mg/kg/day of methionine.

The term “nutritional composition” as used herein is defined as a composition for providing nourishment to an individual with cancer, preferably for enteral administration. In one embodiment, the nutritional composition as a whole is not naturally occurring but is comprised of naturally occurring components, synthetically produced components, or a combination thereof (thus, semi-synthetic). In some embodiments, the nutritional composition comprises elemental amino acids. In additional embodiments, substantially all of the standard elemental amino acids are at RDI levels, which are well known in the art, except for methionine.

The term “organoleptic” as used herein refers to stimulating any of the organs of sensation. In particular, as used herein the term refers to stimulating or improving taste and/or smell.

The term “parenteral” as used herein refers to administration by some means other than through the gastrointestinal tract or lungs, such as intravenous, subcutaneous, intramuscular, or intramedullary injection.

The term “protein system” as used herein refers to any form of protein, peptide, polypeptide, and the like employed to deliver at least one dietary amino acid. For example, in lieu of an actual intact protein, or even a hydrolyzed protein source, an “elemental” formula which supplies the amino acids as “individual, pure, free amino acids” is utilized. The mixture/collection of free amino acids is the protein system for an elemental product. Thus, the protein system could be a 1) intact protein(s); 2) partially hydrolyzed protein(s); 3) completely hydrolyzed protein(s); and/or 4) a composite of free amino acids.

The term “substantially lacking in methionine” as used herein refers to a diet or nutritional composition having zero or extremely low detectable levels of methionine. A skilled artisan recognizes that this amount is less than about 1 mg/kg/day.

In an object of the present invention, there is a cycled regimen for accelerated induction of apoptosis in tumor cells. A methionine level-reducing agent is administered to a mammal with cancer, and in some embodiments, the agent is administered in the absence of other methionine level-reducing agents. In specific embodiments of the present invention, a diet having no or significantly low levels (approximately less than about 1 mg/kg/day, or in some embodiments, less than about 0.5 mg/kg/day) of methionine is administered to a cancer patient, particularly one having metastatic cancer, cancer refractory to standard treatment, and/or cancer in which no effective therapy is known. However, in an alternative embodiment of the present invention, multiple methionine level-reducing agents or associated regimens are administered in addition to the diet, such as administration of homocysteine, administration of low levels of other amino acids, and/or administration of methionine scavenging agents, such as methioninase.

In one embodiment, this diet is administered chronically, with no subsequent cycling regimen. In an alternative specific embodiment, a diet substantially lacking in methionine and/or a methionine-restricted diet are administered prior to administration of a diet replete with methionine. Thus, in one embodiment, a diet substantially lacking in methionine is administered for a time t₁, a methionine-restricted diet is administered for a time t₂, and a diet replete with methionine is administered for a time t₃. In one embodiment, this cycling regimen is repeated at least once in part or in full. A skilled artisan recognizes that the times t₁, t₂, and t₃ may be variable and are determined empirically by standard methods in the art, such as in clinical trials. Furthermore, the length of times t₁, t₂, and t₃ may be dependent on multiple factors, such as a patient's overall state of health, the type of cancer, the stage of the disease, the patient's age and weight, and so forth.

In one embodiment, a chronic diet substantially lacking in methionine or which is methionine-restricted is administered to a cancer-stricken mammal for tumor growth inhibition and extension of life, wherein the administration occurs in the absence of a subsequent methionine replete diet. In one embodiment, the diet consists essentially of a methionine-restricted composition. In another embodiment, the diet comprises a methionine-reduced composition. In some embodiments of the present invention, the composition substantially lacks methionine or is methionine-restricted and is supplemented with whole foods having a limited methionine content to meet a methionine dietary target of about 2 mg/kg/day. In a preferred embodiment for the chronic diet administration, methionine is the sole amino acid present at reduced levels in a balanced elemental amino acid composition. In a specific embodiment, methionine is present at reduced levels in a naturally occurring composition, such as a whole food or purified protein from a food.

In an additional object of the present invention, there is adjuvant therapy with the diet of the present invention to improve the efficacy of a specific chemotherapeutic agent or the efficacy of radiation against a specific cancer type. In one embodiment, the methionine-restricted diet and/or diet substantially lacking methionine is administered and is actively reducing plasma methionine levels before the cancer therapy is administered. A skilled artisan recognizes that this approach renders the tumor cells more susceptible to the additional cancer therapy, such as chemotherapy agents. In one embodiment, the diet is maintained during the additional cancer therapy.

In some embodiments, the additional cancer therapy is chemotherapy, surgery, radiation, gene therapy, immunotherapy, biological therapy, differentiating agents, chemopreventive agents, or a combination thereof. In some embodiments, chemotherapy refers to drugs or agents which are cytotoxic to a cell. Examples include antiangiogenesis agents, telomerase inhibitors, inducers of apoptosis, alkylating agents, plant alkaloids, antibiotics, and the like. A skilled artisan recognizes that immunotherapy for the treatment of cancer may include administration of a vaccine (for example, see Monzavi-Karbassi et al., 2001), administration of compositions comprising monoclonal antibodies (for example, see Weiner, 1999; Goldenberg, 1994), or a combination thereof. A skilled artisan recognizes that biological therapy (Myers, 1999) as used herein refers to such agents as interferon, interleukins, cytokines, and the like. A skilled artisan also recognizes that differentiating agents may be used as a form of cancer therapy, wherein the agents are a form of chemotherapy which are not necessarily cytotoxic to the cancer cell. Examples include phenylacetate, phenylbutyrate, vitamins and their analogs (such as vitamin D or vitamin A), including all trans retinoic acid (such as for leukemia). In some embodiments, the differentiating agents enhance or facilitate differentiation of a cancer cell back to a relatively normal phenotype, such as in retarding its proliferation. A skilled artisan also recognizes that another form of cancer therapy which may be used in conjunction with the methods of the present invention are chemopreventive agents, such as plant-derived agents, including retinoids and their analogs, including synthetic-produced analogs, vitamins and their analogs, and so forth.

In another object of the present invention, there are formulations of the diet with pleasing organoleptic characteristics to improve patient acceptance and compliance for cancer therapy. In a specific embodiment, there is an elemental diet having flavoring/masking agents to enhance organoleptic characteristics. In another specific embodiment, there is a specialized protein having zero or low levels of methionine.

Thus, in accordance with the objects of the present invention, methionine is deprived in a cancer cell. The initial restriction of methionine in the diet will induce cell-cycle arrest for tumor cells in the late S and/or G2 phase (Guo et al., 1993; Lu and Epner, 2000). During this arrest, the cell will accumulate oxidative and other types of damage to its cellular machinery (Endo and Goseki, 1990). More importantly, the methylation status of the tumor DNA will change. This is due putatively to the depletion of the pool of S-adenosylmethionine that acts as the principle methyl-group donor in many metabolic pathways including methylation of DNA. It is known that uncontrolled growth of cancer cells is associated with focal hypermethylation of DNA and resultant transcriptional silencing of several growth inhibitory and proapoptotic genes (Baylin et al., 1998). Methionine restriction causes activation of Jun N terminal kinase (JNK1), a stress activated protein kinase involved in several diverse biological processes. Furthermore, transient transfection of dominant negative JNK1 blocked prostate cancer apoptosis in response to methionine restriction. Thus, JNK1 and/or other stress activated protein kinase cascades are likely required for the observed antitumor activity of methionine restriction.

The Examples provided herein indicate that in humans dietary deprivation of methionine alone is sufficient to exert a direct growth inhibitory effect on advanced tumors and affords an extended lifespan expectancy. Furthermore, the apparent partial regression of an advanced tumor upon discontinuation of a methionine-restricted diet has been observed. This indicates that the cycling of a methionine-depleted diet and a methionine-replete diet is a useful therapy for tumors. Upon repletion with a complete diet, these previously arrested tumor cells again try to progress through the normal cell cycle. However, the damage accumulated during the arrested phase is too great and, instead of normal cell-cycle progression, the repletion of the diet launches these cells towards apoptosis. Very importantly, the methionine restriction has already effected a change in methylation status of the tumor DNA and the induced expression of proapoptotic genes is now possible. The net effect is that dietary methionine restriction, followed by repletion of diet, induces apoptosis in tumor cells.

Although in some embodiments the present invention is useful as a treatment for persons with advanced cancers that have already proven refractory to conventional chemotherapy treatments, in some embodiments the invention would also be effective for early stage tumors. Furthermore, the application of the present invention is not limited to any one particular type/origin of cancer. While studies by the inventors have involved advanced and hormone-independent prostate cancer, there is abundant information from published in vitro (Hoffman et al., 1983) and in vivo (Hoffman et al., 1995) studies indicating that cancers from many origins (lung, breast, colon, glioma, gastric, etc.) are susceptible to the effects of methionine restriction. Thus, the present invention is applicable to a wide range of cancer types from various primary origins. Examples include prostate, lung, breast, colon, glioma, gastric, skin, esophagus, squamous cell carcinoma of head and neck region, pancreas, small intestine, bladder and urinary collecting system, kidney, testes, ovary, soft tissue or osteogenic sarcoma, Hodgkin's lymphoma, non-Hodgkin's lymphoma, leukemia, melanoma, rectum, anus, liver, or brain. In one embodiment, the methods and compositions of the present invention are useful for an early stage cancer. A skilled artisan recognizes that this is tested by administering the diet to individuals having no cancer and measuring their plasma methionine levels. A reduction in circulating methionine levels in the presence of no tumor load indicates that similar responses would be seen in individuals having a small tumor load, and thus in individuals in early stages of cancer.

In a specific embodiment, the therapeutic strategy consists of the following: A severely methionine-restricted diet (no more than approximately 2 mg/kg/day) is first fed to induce cell-cycle arrest. During this arrest, tumor cells experience a reduction in the global methylation status of DNA and their cellular machinery accumulate oxidative and other types of damage. Then, a replete diet is fed to force tumor cells back into cell-cycle progression and subsequent induction of widespread apoptosis. Ultimately this results in the gradual, and possibly full, regression of the tumor. It specific embodiments, further benefit is derived from multiple cycles of methionine-depleted and replete diets.

II. Cancer and Diet: a Specialized Diet as a Therapeutic Agent

A. Inhibition of Tumor Cell Proliferation by Depletion of Methionine as a Metabolic Precursor, via S-Adenosyl Methionine (SAM), for Production of Polyamines

As already discussed, methionine depletion results in strong inhibition of tumor cell growth. Moreover, this inhibition seems to be almost universal to any/all types of tumor cells. There are three likely mechanisms by which this inhibition is effected. The first, and most obvious, is to suppress the expression of various proteins due to the absence of that amino acid as a principle building block for synthesis of proteins. The second is to block the synthesis of various polyamines by removal of methionine as a metabolic precursor. Although the exact mechanism for their action has not been clearly elucidated, the polyamines (e.g., spermine) have been implicated as essential participants in the mitotic process. The third mechanism is a change in the global methylation status (mediated by depletion of the methyl donor S-adenosylmethionine) of the tumor cell's DNA. A change in methylation status can turn on or turn off various growth inhibitory and/or growth promoting genes. It is not necessary to determine which of the aforementioned modes of action is primarily responsible for effecting tumor inhibition. It is likely that all may contribute to the overall level of inhibition observed.

A central tenet undergirding dietary methionine restriction as a therapeutic strategy is “methionine dependence.” Methionine dependence is a tumor-specific biochemical defect expressed by the inability, or decreased ability, of tumors to grow under the conditions of methionine depletion by substitution with homocysteine. Methionine dependence has not been observed in normal cells; which are able to synthesize methionine from other metabolic precursors (e.g., homocysteine). Some tumor cells do not express the enzyme(s) necessary to convert homocysteine into methionine, although others do. Those that do are probably methionine dependent as a result of abnormal methionine utilization. Thus, tumor cells have an absolute requirement for an exogenous source of methionine to meet their elevated metabolic need for methionine.

There are many studies which show that methionine deprivation causes tumor growth inhibition. Previously mentioned were those studies by Millis et al. (1996; 1998) which showed significant tumor growth inhibition (40% vs. control) for subcutaneously transplanted hepatomas in rats. The exact cause and effect are hard to pinpoint in these studies, however, since Millis altered the quantities of both arginine and methionine in various combinations of deficient, normal, and excess dietary levels.

Although nearly all tumor cell lines display an absolute methionine dependence or an elevated methionine dependence, Kreis (1979) suggests this is not unequivocal. Kreis studied 2 normal cell lines and 11 malignant cell lines in vitro, and all were cultured in media containing either methionine or homocysteine. Equal growth occurred in either medium for both normal cell lines (fetal lung fibroblasts and bladder epithelial cells), and there was equivalent growth for 5 of the malignant human cell lines (cervix carcinoma HeLa, breast adenocarcinoma A1Ab, acute lymphoblastic leukemia MOLT-3, Wilm's tumor SK-NEP-1, and reticulum cell sarcoma T-77). An absolute requirement (no growth in homocysteine medium) for 4 of the malignant human cell lines (breast adenocarcinoma SK-BR-2-III, lymphoblastic leukemia CCRF-HSB-2, lymphoblastic leukemia CCRF-SB, and neuroblastoma SK-N-MC) was determined. Finally, Kreis reported restricted growth for 2 of the malignant cell lines (lung adenocarcinoma A549 and pancreas adenocarcinoma Capan-1). Thus, of the 11 malignant cell lines studied, 6 were found to be susceptible to inhibition by methionine deprivation.

B. Methionine Depletion via Enzyme Therapy

Dietary absence of methionine does not result in complete depletion of circulating levels of plasma methionine. This is especially evident in animal studies employing mice and/or rats. Nobori et al. (1997) has shown that feeding mice a diet deficient in methionine, homocysteine, and choline will achieve a 70% reduction in plasma methionine levels. However, this diet is ultimately fatal to mice. In an attempt to mitigate against this fatal consequence, methionine can be replaced by homocysteine (which can be converted into methionine by normal tissue). A diet restricted in choline and where methionine is replaced by an equimolar quantity of homocysteine results in a 50% chronic reduction of plasma methionine.

As an alternative to dietary depletion of methionine, many investigators have studied the potential use of methioninase. Pseudomonas putida is the microbe from which methioninase is most commonly derived. Recombinant methioninase is a homotetrameric pyridoxal 5′-phosphate enzyme with a molecular weight of 172 kilodalton. It is commonly cloned in E coli. As one might infer from the name, this enzyme scavenges/destroys methionine. The circulating half-life of methioninase is approximately 1.5 hours after i.v. injection. In order to prevent immunological reactions which might be produced by multiple dosing of methioninase and to prolong the serum half-life of methioninase, there have been various efforts to conjugate methioninase to PEG. A recent example of this is reported by Hoffman et al. (1998), which indicated that the serum half-life of PEG-methioninase increased to 2.65 hours. No loss of enzymatic activity due to conjugation to PEG is reported.

Methioninase injected at 1,000 U/kg causes an acute reduction of plasma methionine by 80% (Nobori et al., 1997). Recovery of baseline levels of plasma methionine occur within 14 hours. Hoffman et al. (1998) reports an even greater reduction of plasma methionine levels. A single i.p. injection of 300 units of methioninase lowered serum methionine levels in mice from 70 μM to less than 1 μM within 2 hours and maintained this depleted level for 8 hours.

Nobori combined all the above strategies to try to produce a regimen which would drastically lower plasma methionine levels but also not harm the mice. He fed a diet restricted in methionine, homocysteine, and choline. In addition to this, he administered synchronous treatments of intraperitoneal injections of methioninase (1,000 U/kg) and homocystine (50 mg/kg). Each was administered at 12-hour intervals. Nobori reports this resulted in steady-state levels of plasma methionine at <5 μM. Additionally, Nobori claims that no deleterious side-effects (including weight loss or liver pathology) was evidenced.

Hoffman et al. (1997) reports the results of a pilot Phase I clinical trial to determine the toxicity and pharmacokinetics of recombinant methioninase. Levels of serum methionine-depletion in cancer patients are also reported. Patients with advanced cancers were given methioninase in single-dose treatments administered by i.v. infusion. No clinical toxicity is reported and serum-methionine levels were reported as low as 0.1 μM. There is no mention of any immune response, since this is a single-dose experiment. However, this must be a problem since they report more recent efforts to conjugate methioninase to PEG in order to reduce its immunogenicity (Hoffman et al., 1998).

III. Dietary Products as Chemotherapy Adjuvants—Methionine Depletion Stresses Tumor Cells and Results in Increased Effectiveness of Various Chemotherapies

As has already been mentioned, methionine starvation leads to depleted methionine levels in tumor cells and modifies methylation reactions by modulating levels of SAM. Another important effect of methionine starvation is lowered levels of intratumoral glutathione (42% decrease (Endo and Goseki, 1990)). This is especially important regarding drug resistant phenotypes. High levels of intracellular glutathione, leading to increased antioxidant activities, contributes to the expression of many drug resistant phenotypes. Glutathione-mediated detoxification of free radicals and toxic electrophiles has been implicated in resistance of tumor cells to numerous chemotherapeutic agents. Studies have demonstrated that this glutathione-mediated drug resistance can be reversed by buthionine sulfoximine (BSO), which specifically blocks y-glutamyl cysteine synthase, a key enzyme in the synthesis of glutathione. Thus, methionine starvation might result in increased effectiveness of certain chemotherapeutic agents due to suppression of intracellular glutathione levels. In fact, one might reasonably predict that whenever coadministration of BSO results in increased effectiveness of a chemotherapeutic agent, a regimen of methionine (and/or cyst(e)ine) restriction will also increase the effectiveness of that same chemotherapy drug. This is particularly exciting since many chemotherapy drugs have been reported to exhibit enhanced activity when combined with BSO.

There are studies in animals which document increased efficacy of chemotherapeutic agents when administered concurrent with a regimen of methionine depletion. Goseki et al. (1992) reports that a methionine depleting TPN diet improves the effectiveness of doxorubicin administered to Yoshida sarcoma-bearing rats. Rats were fed for eight days on TPN, and doxorubicin was administered on days 2, 4, and 6 at a dose of 1.0 mg/kg/day.

An enhanced effectiveness of doxorubicin occurred when coadministered with a methionine depleted diet through reduced tumor weight and increased survivability.

Other drugs have been shown to exhibit enhanced tumor toxicity when administered concurrent with a methionine depleting diet. Hoffman et al. (1997) reports a study involving human gastric cancer (SC-1-NU) xenografts in nude mice. Methionine depleted diet combined with administration of 5-fluorouracil (at a dose of 30 mg/kg) enhanced antitumor activity of 5-fluorouracil by approximately twofold (p<0.05). Hoffman et al. (1996) also reports the enhanced effect of cisplatin when combined with methionine depletion. When treating a human breast carcinoma (MX-t) cell line in nude mice, methionine-depleted diet alone is reported to have very little effect. However, the combination is reported to show significant tumor growth inhibition. Treated tumor growth averaged only 32% of the growth measured in control mice tumors.

An interesting strategy to more fully utilize methionine-depleted diets is reported by Yoshida et al. (1998), who conjugated a chemotherapeutic drug, mitomycin C, to methionine and then administered this drug while feeding a methionine-depleted diet. When combined with the nutritional therapy, the methionine-conjugated version of the drug was significantly more effective at tumor growth inhibition than was the plain mitomycin C. Thus, the combination of the diet plus methionine-conjugation of the drug seemed to more selectively target the tumor issue.

Finally, in some embodiments the mode of action for enhancement of drug therapy by methionine depletion involve mechanisms other than glutathione suppression. Although methionine starvation leads to depleted methionine levels in cells, modifies methylation reactions, lowers glutathione levels, alters folate distribution and leads to cell-cycle arrest in the late S phase, a combination of these mechanisms may be responsible. In a specific embodiment, cell-cycle arrest, leading to phase alignment, is used to enhance the effectiveness of drugs that are specifically intended to be administered during, and interfere with, the mitotic process.

IV. Methionine Level-Reducing Agents

The present invention is directed to depleting methionine levels in a cell of an individual afflicted with cancer, followed by repletion of the methionine levels. Thus, a patient is subjected to a cycling regime of methionine depletion and methionine repletion. A skilled artisan recognizes, based on the teachings provided herein and on numerous other references in the art, that there are a number of ways to achieve methionine depletion.

The reduction or depletion of methionine levels is facilitated by administration of a methionine level-reducing agent. A skilled artisan recognizes that the term methionine level-reducing agent refers to a composition which reduces the methionine levels in the organism to which it is administered to. The methionine levels may be reduced in the tumor, in the plasma of the organism, or both. In some embodiments, the methionine level-reducing agent is comprised in a composition which targets a tumor, thereby reducing the methionine levels to the tumor alone. For example, a methionine level reducing agent may be conjugated or associated with an anaerobic bacteria, and following administration of the agent/bacteria composition the necessity for the bacteria to grow in hypoxic conditions of a tumor results in targeting of the composition directly to the tumor (Fox et al., 1996; Lemmon et al., 1997; Yazawa et al., 2000). Alternatively, the methionine level reducing agent may be linked, bound, or otherwise associated with a moiety which targets the methionine level reducing agent to the tumor, such as an antibody (Weiner, 1999; Goldenberg, 1994), or a peptide (for example, see Koivunen et al., 1999; Hoppe-Seyler and Butz, 2000).

The diets of the present invention are developed concerning desirable qualities, such as organoleptic characteristics, safety for consumption, and so forth. Diets which comprise amenable taste, smell and feel are desirable. A skilled artisan recognizes which food additives, flavor enhancers, preservatives and the like are useful, particularly following standard testing means. The better the organoleptic characteristics, the higher the compliance of the individual. A skilled artisan also recognizes that a database of known food additives is available from the United States Food and Drug Administration. This is an informational database maintained by the U.S. Food and Drug Administration (FDA) Center for Food Safety and Applied Nutrition (CFSAN) under an ongoing program known as the Priority-based Assessment of Food Additives (PAFA). It contains administrative, chemical and toxicological information on over 2000 substances directly added to food, including substances regulated by the U.S. Food and Drug Administration (FDA) as direct, “secondary” direct, and color additives, and Generally Recognized As Safe (GRAS) and prior-sanctioned substances. In addition, the database contains only administrative and chemical information on less than 1000 such substances. The more than 3000 total substances together comprise an inventory often referred to as “Everything” Added to Food in the United States (EAFUS).

A. Elemental Amino Acid Compositions

In the manufacture of an anticancer enteral feeding composition of the present invention for oral feeding, the incorporation of non-essential amino acids such as alanine, glycine, asparagine, glutamine, proline and serine is favorable to the taste of the composition and, therefore, desirable. The amino acids incorporated into the elemental amino acid composition for the methionine-restricted diet, in some embodiments, are in appropriate proportions so as to constitute a balanced nutritional composition for the human.

In some embodiments, compositions such as Hominex-2® and “Tumorex” (see Tables 1, 2, and 3) are utilized in the diets of the present invention. In a specific embodiment, Hominex-2® formula is a product used for diets which would be supplemented by foodstuffs which are naturally very low in methionine. Thus, the Hominex-2® would serve as a base for an expanded diet. Although the overwhelming majority of the dietary amino acids would come from the Hominex-2® composition, a significant amount of the diet's overall calories would be supplied by other augmented foodstuffs, in some embodiments. This formula is one product which could be used for chronic therapy for tumor growth inhibition.

In another embodiment, Tumorex is a sole-source feeding product, having two embodiments. In the first embodiment, the composition is completely absent of methionine. In a second embodiment (illustrated in Tables 1 and 2), the product has a low level of methionine added which would supply the patient with approximately 2 mg/kg/day (methionine restricted diet).

TABLE 1
Grams Amino Acids Delivered per 2,000 Calories Consumed

| | Old | New Hominex- | |
|---|---|---|---|
| | Hominex-2 | 2 | Tumorex |
| Alanine | 16.4 | 11.0 | 6.4 |
| Arginine | 11.4 | 11.4 | 6.7 |
| Asparagine | 0.0 | 7.8 | 4.6 |
| Aspartic Acid | 8.3 | 1.3 | 1.2 |
| Cyst(e)ine | 5.0 | 5.0 | 1.5 |
| Methionine | 0.0 | 0.0 | 0.15 |
| Glutamine | 0.0 | 13.4 | 12.6 |
| Glutamic Acid | 12.4 | 2.2 | 2.3 |
| Glycine | 18.4 | 11.0 | 6.4 |
| Histidine | 4.6 | 4.6 | 2.7 |
| Isoleucine | 11.9 | 11.9 | 5.4 |
| Leucine | 18.4 | 18.4 | 10.8 |
| Lysine | 11.0 | 11.0 | 6.5 |
| Phenylalanine | 9.7 | 9.7 | 4.7 |
| Tyrosine | 9.7 | 9.7 | 4.7 |
| Proline | 15.8 | 14.3 | 8.4 |
| Serine | 8.2 | 8.2 | 4.8 |
| Threonine | 7.7 | 7.7 | 4.5 |
| Tryptophan | 1.9 | 1.9 | 1.1 |
| Valine | 13.4 | 13.4 | 6.8 |
| | 184.2 | 173.9 | 102.3 |

TABLE 2
Grams Amino Acids Per 100 Grams Powder Product

| | Old | New Hominex- | |
|---|---|---|---|
| | Hominex-2 | 2 | Tumorex |
| Alanine | 3.4 | 2.3 | 1.3 |
| Arginine | 2.3 | 2.3 | 1.4 |
| Asparagine | 0.0 | 1.6 | 0.9 |
| Aspartic Acid | 1.7 | 0.3 | 0.2 |
| Cyst(e)ine | 1.0 | 1.0 | 0.3 |
| Methionine | 0.0 | 0.0 | 0.03 |
| Glutamine | 0.0 | 2.7 | 2.6 |
| Glutamic | 2.5 | 0.5 | 0.5 |
| Acid | | | |
| Glycine | 3.8 | 2.3 | 1.3 |
| Histidine | 0.9 | 0.9 | 0.6 |
| Isoleucine | 2.4 | 2.4 | 1.1 |
| Leucine | 3.8 | 3.8 | 2.2 |
| Lysine | 2.3 | 2.3 | 1.3 |
| Phenylalanine | 2.0 | 2.0 | 1.0 |
| Tyrosine | 2.0 | 2.0 | 1.0 |
| Proline | 3.2 | 2.9 | 1.7 |
| Serine | 1.7 | 1.7 | 1.0 |
| Threonine | 1.6 | 1.6 | 0.9 |
| Tryptophan | 0.4 | 0.4 | 0.2 |
| Valine | 2.7 | 2.7 | 1.4 |
| | 37.8 | 35.6 | 21.0 |

TABLE 3
Quantities Per 100 Grams Powder Product

| | Old Hominex- | New Hominex- | |
|---|---|---|---|
| | 2 | 2 | Tumorex |
| Protein equivalent, g | 30.0 | 30.0 | 18.0 |
| Fat, g | 15.5 | 14.0 | 14.0 |
| Carbohydrate, g | 30.0 | 35.0 | 48.0 |
| Water, g | 1.0 | 1.0 | 1.0 |
| Calories | 410.0 | 410.0 | 410.0 |
| Carnitine, mg | 40.0 | 40.0 | 40.0 |
| Taurine, mg | 60.0 | 50.0 | 50.0 |
| Vitamin A, μg (RE) | 660.0 | 660.0 | 660.0 |
| Vitamin D, μg | 9.9 | 7.5 | 7.5 |
| Vitamin E, mg (alpha- | 12.1 | 12.1 | 12.1 |
| TE) | | | |
| Vitamin K, μg | 60.0 | 60.0 | 30.0 |
| Thiamin, mg | 4.0 | 3.3 | 1.6 |
| Riboflavin, mg | 1.8 | 1.8 | 1.8 |
| Vitamin B6, mg | 1.3 | 1.3 | 1.3 |
| Vitamin B12, μg | 9.5 | 5.0 | 5.0 |
| Niacin, mg (NE) | 26.0 | 21.7 | 11.0 |
| Folic acid, μg | 430.0 | 450.0 | 225.0 |
| Panthothenic acid, mg | 14.0 | 8.0 | 8.0 |
| Biotin, μg | 130.0 | 100.0 | 100.0 |
| Vitamin C, mg | 60.0 | 60.0 | 60.0 |
| Choline, mg | 100.0 | 100.0 | 100.0 |
| Inositol, mg | 70.0 | 70.0 | 70.0 |
| Calcium, mg | 880.0 | 880.0 | 440.0 |
| Phosphorus, mg | 880.0 | 760.0 | 760.0 |
| Magnesium, mg | 225.0 | 225.0 | 112.5 |
| Iron, mg | 13.0 | 13.0 | 7.5 |
| Zinc, mg | 13.0 | 13.0 | 7.5 |
| Manganese, mg | 0.6 | 0.8 | 0.8 |
| Copper, mg | 1.0 | 1.0 | 1.0 |
| Iodine, μg | 100.0 | 100.0 | 100.0 |
| Selenium, μg | 27.0 | 35.0 | 35.0 |
| Chromium, μg | 0.0 | 27.0 | 27.0 |
| Molybdenum, μg | 0.0 | 30.0 | 30.0 |
| Sodium, mg | 880.0 | 880.0 | 440.0 |
| Potassium, mg | 1370.0 | 1370.0 | 700.0 |
| Chloride, mg | 1160.0 | 1160.0 | 600.0 |

The “new Hominex-2®” composition described above is a reformulated version from the “old Hominex-2®” to provide improved taste characteristics.

B. Recombinant Technology

Methods to generate a genetically altered gene producing a protein with the desired methionine level are well known. In fact, this approach has been documented for other amino acids as exemplified by U.S. Pat. No. 6,004,930, which is directed to treatment of phenylketouria by development of a protein with no phenylalanine.

Further refinements to this approach, such as adding a tag to the protein to aid in the purification of the recombinant protein (Yang et al., 1997; Evans, 1998) or inducing the recombinant protein to be secreted (Anram and Smith, 1998; Bingle et al., 2000) are known to those who practice the art.

C. Chemical or Enzymatic Treatment

Chemicals and enzymes able to alter the methionines of intact proteins, such that they are rendered unavailable to the body's metabolism, are available. However, drawbacks do exist. Chemical treatments sufficiently potent to “remove” the desired amount of methionine from protein sources would likely affect other amino acids as well. In addition, the validated removal of the chemicals prior to formulation of the nutritional product would have to be undertaken. The use of suitable enzyme(s), which would likely be more specific and controllable, would require the identification of a Generally Recognized as Safe (GRAS) source for the enzyme(s) or validated removal of the enzyme(s) prior to formulation.

D. Purification and/or Enrichment of Low-Methionine Protein(s)

In one embodiment of the present invention, an intact protein, such as from a whole food, is purified or isolated and utilized as at least a part of a methionine restricted diet or a diet substantially lacking in methionine. The term “intact protein” as used herein refers to a protein preferably not subjected to either chemical or enzymatic hydrolysis, and preferably is in a form similar or identical to its natural state. A skilled artisan recognizes that, although a hydrolyzed or otherwise fragmented protein may be more easily digestable, it would have poorer organoleptic qualities, and therefore likely result in a reduction in compliance of diet consumption of the cancer-affected individual.

In a specific embodiment, the intact protein is obtained from a legume. The seed storage proteins of legumes, especially garden peas and broad beans, are strikingly low in methionine. As noted above, vicilins from the garden pea are totally lacking in methionine. Garden peas and/or broad beans would appear to be cost feasible sources, as both are widely and extensively cultivated. Moreover, their seeds contain significant levels of protein (20 to 25% by dry weight). With regard to purification, the low methionine seed storage proteins of the garden pea and broad bean are classified as globulins, which are defined as proteins soluble in salt solutions but insoluble in water. These solubility characteristics have been used to isolate globulins for over a century (Osborne and Campbell (1898)). While the methods used are not amenable to large-scale production, the underlying solubility characteristics could be utilized in large-scale production. U.S. Pat. No. 5,208,039 states “The source of amino acids other than homocysteine can, in such case, be composed partially or solely of proteins which are low in methionine, for example, soya proteins or soya protein hydrolyzates.” However, as envisioned by the treatment protocol of the present disclosure, the soya proteins or their hydrolyzates contain unacceptably high levels of methionine. U.S. Pat. No. 5,817,695 also proposes a diet restricted in methionine for the treatment of cancer but apart from stating the proteins of the product should possess L-methionine in an amount from 5 to about 11 wt. %, the patent makes no claims or suggestions as to the source of these proteins.

E. Complete Food(s)

The USDA Nutrition Database for Standard Reference allows the calculation of methionine as a weight percent of total protein. Four foods have been identified with methionine levels of 0.1% or less, six with levels of 0.1 to 0.2% and five with levels of 0.2 to 0.3%. Other databases may be searched by methods known in the art to expand the list of foods considered. While the use of complete foods would eliminate the need for purification of proteins, processing would still be required to produce an acceptable nutritional component as the removal of excessive fiber, carbohydrate, minerals, and such. from the foods would likely be required. Also, a skilled artisan recognizes that other essential dietary components (e.g. vitamins) would likely need to be supplemented to meet RDI.

V. Seed Storage Proteins with Reduced Levels of Methionine

In a specific embodiment of the present invention, a methionine level-reducing agent is administered to a cancer-stricken individual as a diet, wherein at least part of the diet stems from seed storage proteins having reduced levels of methionine.

A. Introduction

Seed storage proteins, especially those of legumes, typically contain low levels of the sulfur containing amino acids cysteine and methionine. These proteins are usually sequestered in membrane bound vesicles termed protein bodies. In legumes, which are dicotyledons, the seed storage proteins are found in the cotyledons. Seed storage proteins are classified as albumins (soluble in water and dilute buffers at neutral pH) or globulins (soluble in salt solutions but insoluble in water). Globulins generally contain a lower percentage of methionine than do the albumins. Seed storage globulins can be further categorized by their sedimentation behavior into 11S, 7S and 2S proteins with the 2S proteins being a very minor component. Within the globulins, the 7S proteins typically have lower levels of methionine than do the 11S proteins (Casey et al., 1986; Bewly and Black, 1994). The names assigned to globulins from various sources and their methionine content are given in Table 4.

TABLE 4
Seed Storage Proteins with Mole Percent Methionine Content

| | Globulins | |
|---|---|---|
| Protein Source | 11S proteins | 7S Proteins |
| Maize | | globulin 1 (0.4%) |
| Alfalfa | medicagins | aflins |
| Rapeseed | cruciferans (1.2%) | napins (2.3%) |
| Peanut | | ARA H1/H2 (1%) |
| Mung Bean | | vicilins (0.45%) |
| Jack Bean (Horse Bean) | | canalins (0.9%) |
| Sword Bean (Japanese Jack | | canalins (0.9%) |
| Bean) | | |
| Kidney Bean | | phaseolins (0.8%) |
| Soybean | glycinins (1%) | β-glycinins (0.3%) |
| Broad Bean | legumins (0.2%) | vicilins (0.1%) |
| Garden Pea | legumins (0.5%) | vicilins (0%) and |
| | | convicilins (0.1%) |

The holoprotein of the 11S globulins of the pea and broad bean is a hexamer (Mᵣ 360,000) composed of subunits of approximately Mᵣ 60,000 held together by hydrogen bonds. Each of the (Mᵣ 60,000) subunits is composed of an acidic (Mᵣ 40,000) and a basic (Mᵣ 20,000) polypeptide joined covalently via a disulfide bridge. The acidic and basic polypeptides of each subunit are encoded within the same gene. Processing of the prolegumins, which includes proteolytic cleavage to produce the acidic and basic polypeptides, begins while the protein is still associated with the rough endoplasmic reticulum and is completed within the protein bodies of the cotyledons. A multigene family encodes legumins. The exact number of genes has not been determined but evidence suggests that 22 may not be an unreasonable number. These genes appear to have arisen from a single ancestral gene and significant homology remains.

The holoprotein of the 7S globulins of the pea and broad bean is a trimer (Mᵣ 150,000 to 210,000) whose subunits range in size from Mᵣ 50,000 to Mᵣ 70,000. For the pea, the Mᵣ 50,000 subunits have been termed vicilins and the Mᵣ 70,000 subunits have been termed convicilins. At least for the pea (one of the most extensively studied seeds with regard to the 7S proteins), the abundance of the two 7S proteins appears to be approximately equal on a weight basis. Typically, the differentiation of convicilin from vicilin is not made and the two are referred to as vicilin. Part of this inclusive nature of the nomenclature is historical and part is due to the effort required to separate the two 7S proteins from each other as compared to the effort required to separate the 7S and 11S proteins. The Mᵣ 70,000 polypeptides are very homologous to the Mᵣ 50,000 polypeptides with the difference being an extension from the amino terminal ends of the Mᵣ 50,000 polypeptides. In the pea, vicilins (Mᵣ 50,000) have no methionine residues while the convicilins (Mᵣ 70,000) have a single methionine residue. As with the 11S proteins, the 7S subunit precursors are products of a multigene family with some studies suggesting up to 18 genes. Again, as with the 11S proteins, processing occurs both while the proteins are still associated with the rough endoplasmic reticulum and in the protein bodies. Unlike the 11S proteins, the 7S proteins contain little, if any, cyst(e)ine and hence there are no covalent links between the subunits. The 7S proteins also differ from the 11S proteins in that they are glycosylated. For the pea the level of glycosylation is about 0.6% by weight with the sugars being equally distributed between mannose and glucosamine.

For the garden pea, both the 7S and 11S proteins undergo proteolytic degradation within the protein bodies such that by SDS-PAGE subunits of 12, 14, 18, 24, 30, 47, 50 and 75 kD are found for the 7S proteins (vicilins and convicilins) while subunits of 18, 20, 25, 27, 37 and 40-kD are found for the 11S proteins (legumins). The 20, 40, 50 and 75 kD are the dominant species. Proteolytic degradation is not as extensive in the broad bean as it is in the pea. Vicilins exhibit subunits of 31, 33, 46 and 56 kD while the legumins appear to be present only as intact molecules and exhibit subunits of 20 and 37 kD. The various proteolytic products of the legumins and vicilins of the garden pea and broad bean remain associated with their originating structures (7 or 11S) during isolation of the storage proteins (see below). It is only through the use of SDS-PAGE that the proteolytic fragments can be observed.

Seed storage proteins accumulate during seed development for the cotyledons of the broad bean. A similar accumulation, although with a differing time frame, occurs for the storage proteins of the pea (Gatehouse et al., 1982; Bewly and Black, 1994).

B. Isolation of Seed Storage Proteins

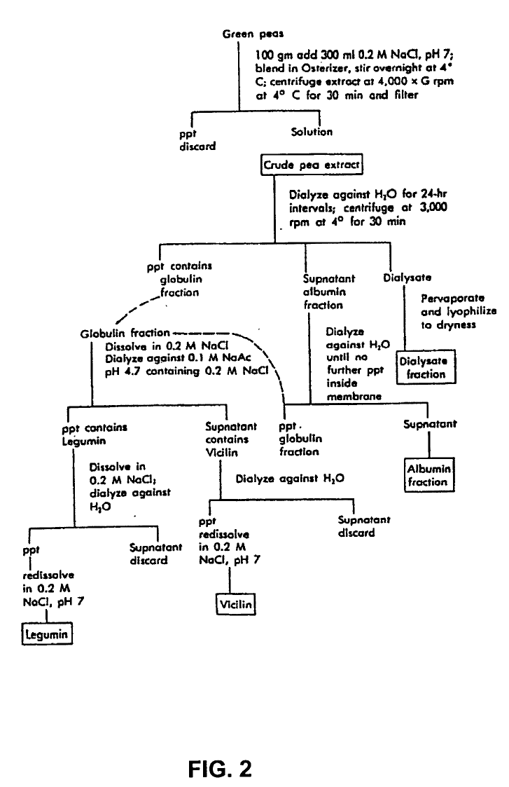

Albumins can be separated from globulins based on the former's solubility in water and dilute buffers at neutral pH. Globulins can be further separated into 11S (legumins in pea and broad bean) and 7S (vicilins, including convicilins, in pea and broad bean) by isoelectric precipitation of the 11S proteins at pH 4.7 to 5.0 in the presence of 0.2 M NaCl. FIG. 2 (Malley et al., 1975) is a schematic of a classical procedure used to isolate the various seed storage fractions from pea and broad bean. The vicilin fraction denoted in the scheme contains both vicilin and convicilin.

Briefly, the scheme involves homogenization of seeds in 0.2 M NaCl, pH 7.0 at 4° C., extraction at 4° C. for 24 hours followed by removal of the insoluble material by centrifugation and filtration. The filtered supernatant, which contains albumins, vicilins and legumins, is dialyzed against water to cause precipitation of the globulins, which are collected in the dialysis bag and are recovered by centrifugation. The globulins are dissolved in 0.2 M NaCl, pH 7.0 and then dialyzed against a buffered solution at pH 4.7 containing 0.2 M NaCl, which induces precipitation of the legumins. The precipitated legumins are recovered by centrifugation. The supernatant from this centrifugation is dialyzed against water to precipitate the vicilins, which are recovered by centrifugation.

The vicilin fraction from pea isolated from a similar scheme contains both vicilin (0% Met) and convicilin (0.1% Met). The two can be separated by ammonium sulfate precipitation followed by centrifugation to recover the vicilin and then dialysis to remove the ammonium sulfate. However, as the methionine target level for the protein source being developed is 0.2%, this separation of vicilin and convicilin will likely not be necessary as the methionine level in the fraction containing both vicilin (0% Met) and convicilin (0.1% Met) should be 0.05% as the two are present in near equal amounts.

While the scheme does produce high purity fractions, the recovery of vicilin is less than 40 to 50% and the use of dialysis obviously is not amenable to large scale (or even pilot scale) production. An isolation scheme based on the scheme described above and involving centrifugation, but not dialysis, could be easily adapted by a skilled artisan, especially with respect to centrifugation conditions and the stirring times required to produce the highest yield and purest form of the vicilin preparation.

A further improvement, with respect to scalability, could utilize filtration, not centrifugation, as the basis for isolation of vicilins. Using this approach, the seeds would be extracted in water and then passed through a filter press that had been precoated with an appropriate filter aid. The press would be washed with chilled water at pH 7.0 to remove albumins. The filter press would then be washed with 0.2M NaCl, pH 4.2 to 5.2 to solubilize the vicilins, leaving the debris and legumins within the filter press. The vicilin fraction would require diafiltration to remove salt.

It is also possible that a micro/ultrafiltration system could be utilized in place of the filter press system to eliminate the filter aid requirement. As noted above, legumins form hexamers of Mᵣ 360,000, and in the insoluble state these hexamers would very likely form aggregates and thus be even larger in size. Filtration cartridges with pores exhibiting nominal cutoffs from 200,000 daltons through 0.65 μm (≈1,000,000 daltons) and with feed channels of up to 3 mm are available. The use of micro/ultrafiltration would eliminate the need for filter aid. However, the initial capital costs would likely be greater and the volumes of liquid generated would likely be larger than for isolation using the filter press approach.

C. Initial Investigations

A good choice as the source of the low-methionine protein preparation would appear to be the garden pea (Pisum sativum). The 7S fraction, containing vicilins, which have no methionines, and convicilins, which have methionine levels of 0.1% (by mole percent), would have an average methionine content of 0.05% (by mole percent). The most likely contaminant of the vicilin fraction would be legumins, which have a methionine content of 0.5% (by mole percent). As the target level of methionine is 0.2%, the vicilins could be contaminated with up to 30% legumins and still meet the target specification.

“Propulse” a commercially available protein fraction from yellow Canadian peas (Parrheim Foods) was obtained. In addition, flour from Kroger split green peas and Renee's Garden snow peas was generated in-house. The three preparations were analyzed for ash, fat, moisture, protein and amino acid composition (Table 5). The typical Parrheim analysis was supplied by the manufacturer.

TABLE 5
Proximate and Amino Acid Analysis of Green, Snow and Yellow Pea Seeds

| | | | RENEE'S | | PROPULSE YELLOW | | |
|---|---|---|---|---|---|---|---|
| | KROGER SPLIT | | GARDEN | | CANADIAN | | |
| | GREEN PEAS | | SNOW PEAS | | PEA PROTEIN | | |
| | raw data | | raw data | | raw data | | Typical |
| | (g/100 g | g/100 g | (g/100 g | g/100 g | (g/100 g | g/100 g | Parrheim |
| Analysis | powder) | protein | powder) | protein | powder) | protein | Analysis |
| Ash | 2.61 | | 3.50 | | 5.35 | | <4.00 |
| Fat | 1.68 | | 1.90 | | 2.36 | | <3.00 |
| Moisture | 10.5 | | 9.12 | | 3.60 | | 4.00-6.00 |
| Protein | 22.1 | | 24.8 | | 81.7 | | 83.0-87.0 |
| asp | 2.51 | 11.36 | 2.75 | 11.09 | 9.90 | 12.12 | 12.43 |
| thr | 0.823 | 3.72 | 0.946 | 3.81 | 2.92 | 3.57 | 4.34 |
| ser | 1.08 | 4.89 | 1.17 | 4.72 | 4.37 | 5.35 | 4.80 |
| glu | 3.74 | 16.92 | 4.04 | 16.29 | 14.4 | 17.63 | 13.74 |
| pro | 0.897 | 4.06 | 0.991 | 4.00 | 3.48 | 4.26 | 5.29 |
| gly | 0.966 | 4.37 | 1.11 | 4.48 | 3.26 | 3.99 | 4.64 |
| ala | 0.966 | 4.37 | 1.16 | 4.68 | 3.42 | 4.19 | 5.04 |
| val | 0.966 | 4.37 | 1.10 | 4.44 | 3.91 | 4.79 | 5.27 |
| met | 0.211 | 0.96 | 0.240 | 0.97 | 0.844 | 1.04 | 1.31 |
| ile | 0.882 | 3.99 | 0.961 | 3.88 | 3.73 | 4.57 | 5.59 |
| leu | 1.59 | 7.19 | 1.75 | 7.06 | 7.04 | 8.62 | 8.44 |
| tyr | 0.786 | 3.56 | 0.909 | 3.67 | 3.00 | 3.67 | 3.12 |
| phe | 1.01 | 4.57 | 1.13 | 4.56 | 4.43 | 5.41 | 6.13 |
| his | 0.52 | 2.35 | 0.571 | 2.30 | 1.81 | 2.22 | 2.52 |
| lys | 1.59 | 7.19 | 1.83 | 7.38 | 5.99 | 7.33 | 6.82 |
| arg | 2.01 | 9.10 | 2.30 | 9.27 | 7.04 | 8.62 | 89.71 |
| cys | 0.284 | 1.29 | 0.305 | 1.23 | 0.428 | 0.52 | 0.76 |
| trp | 0.161 | 0.73 | 0.203 | 0.82 | 0.493 | 0.60 | 1.06 |

The methionine levels of the three preparations ranged from 0.96 (snow peas) to 1.04 (yellow peas) g/100 g of protein. The values stated are the sum of methionine, methionine sulfoxide and methionine sulfone determined from amino acid analysis. The distribution of the remaining amino acids was very similar for the snow and split green peas. The distribution determined for the yellow Canadian peas was slightly different from the other two and moreover, was somewhat different from that supplied by the manufacturer.

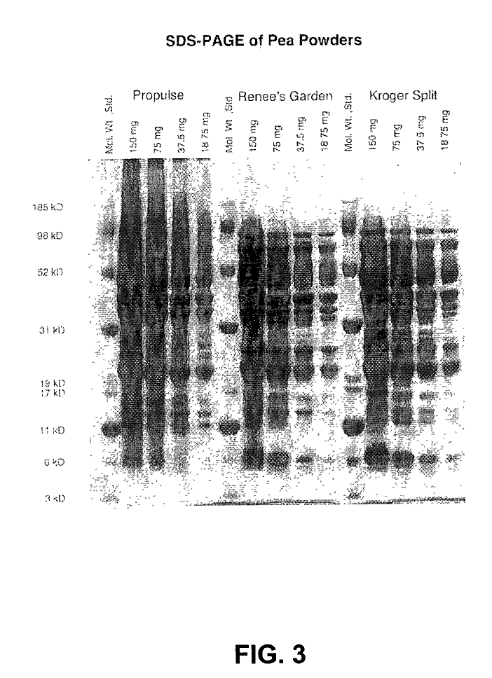

The three preparations were analyzed by SDS-PAGE (FIG. 3). Using the protein contents determined by proximate analysis, equal protein loads of all three were run. The overall band pattern was similar for the three with the Renee's Garden snow peas and the Kroger split green peas exhibiting a virtually identical banding pattern. As evidenced by the “smearing” of proteins in the lanes and stainable material that remained in the loading wells for the Propulse, commercial preparation of the fraction has apparently led to protein denaturation.

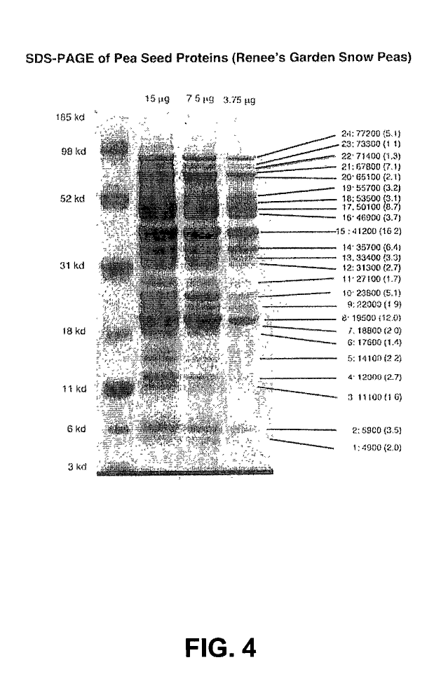

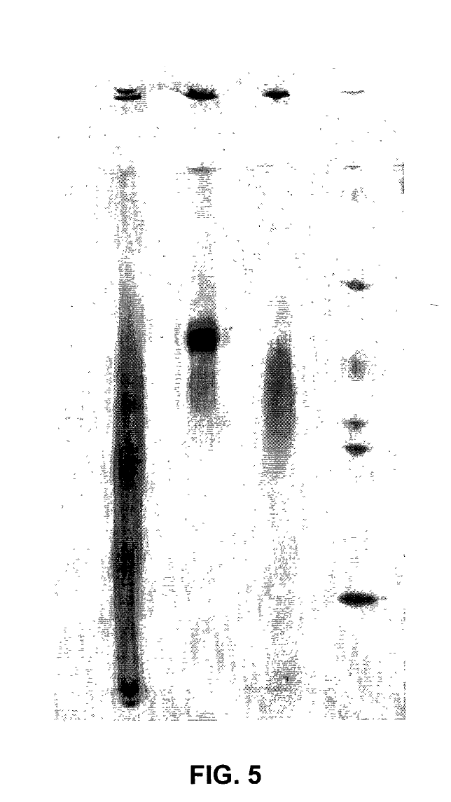

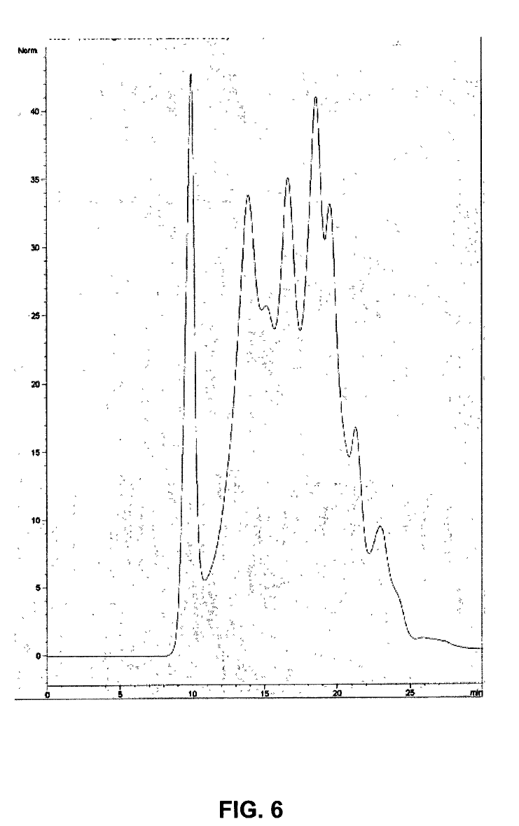

Although amino acid composition analysis is the final analytical criteria for the successful production of a low-methionine protein, more rapid analytical techniques are required for in-process monitoring. Polyacrylamide gel electrophoresis (PAGE), either sodium dodecyl sulfate (SDS) or native as well as size exclusion chromatography (SEC) have all proven useful. SDS-PAGE generates the most detailed analysis (FIG. 4), but this very detail can make interpretation of the results difficult. In FIG. 4, bands 16, 17, 18 and 19 were identified as vicilins, while bands 7, 8, 14, 15, 10, 21 and 22 were identified as legumins. A much simpler protein banding pattern is observed with native (non-reducing and non-denaturing) PAGE as evidenced in FIG. 5. An isolated vicilin fraction is seen as a broad band of material from approximately 150 to 230 kD, while the legumin fraction is seen as a tight band of approximately 250 kD. It should be noted that the legumin fraction does contain some vicilin. SEC can also be used to monitor the seed storage proteins as shown in FIG. 6. The legumin peak is denoted by the letter A, and the vicilin peak is denoted by the letter B.

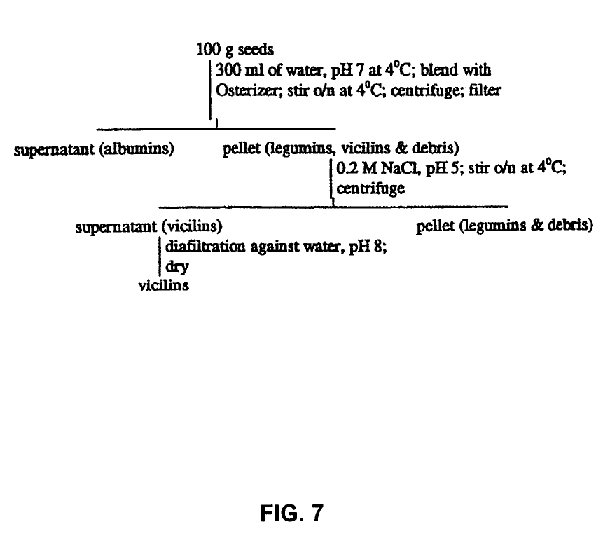

The isolation of vicilin using the scheme of FIG. 7 was next attempted using flour prepared from the green split peas. Lack of solubility was not a problem as deionized water was able to extract not only albumins but vicilins and legumins as well. Even if the protein concentration was increased to such an extent that some albumins were insoluble, vicilins and legumins were still taken up by the deionized water. It has been suggested that the solubility characteristics of the vicilins and legumins is to some extent a function of the route used to achieve solubility. Thus, it may be necessary to extract all proteins with a high salt treatment and then lower the salt concentration in conjunction with alteration of pH to induce specific precipitation of the storage proteins. Although this approach of extracting all proteins and then selectively precipitating the different classes is not as amenable to scale up, it is still technically feasible. Another possible explanation for the extraction of pea globulins by water is a much lower than expected NaCl requirement for solubility. While the literature indicates that concentrations of NaCl of 0.1M or greater are required for solubility of the globulins, some investigations indicate that concentrations as low as 2 μM may be sufficient to solubilize the majority of the globulin fraction. This very low level of NaCl would be present during water extraction as a consequence of the endogenous salt within the pea seeds.

D. Other Considerations

Cultivar or strain variation as well as growth conditions are known to alter the total protein content of seeds as well as the relative ratios of the 7S and 11 S proteins. This in turn can affect the methionine levels found in seeds. In the most thorough study to date (Schroeder, 1982) wild, field and round and wrinkled garden peas were grown under uniform conditions and the seeds examined for protein characteristics. Protein varied from 18 to 32% of the weight of the seed while the globulin (legumin+vicilin) to albumin ratio varied from 1.88 to 3.81. The vicilin to legumin ratios were observed to vary between 0.82 and 3.13. Thus judicious choice of pea strain can have a significant effect of the degree of protein purification required to achieve the targeted methionine levels.

While the seed storage proteins of the pea are favorable for the preparation of a low-methionine protein, the broad bean (Vicia faba) are also utilized. Although the methionine content of its vicilin fraction is 0.1%, compared to the 0.05% for the pea, its legumin fraction contains only 0.2% methionine. Thus, the overall methionine content of the broad bean globulin fraction is less than the pea, which could potentially impact the degree of purification required. As the most difficult separation of the process would be that of vicilin from legumin, elimination of this step is desirable. As with the pea seeds, cultivars of the broad beans might display varying methionine levels. One potential drawback may be the low level of broad bean cultivation in North America and how that might affect availability and pricing. Flour from three strains of broad bean seeds was prepared and submitted for proximate and amino acid composition analysis. The protein content of the three beans was 25% (w/w), which is identical to that for the garden pea.

Legumes, including peas and beans, contain polyphenols, especially tannins that can act to reduce digestibility of proteins (Salunke et al., 1982). This reduction in digestibility appears to be due to a combination of the tannins binding to and thus inactivating enzymes of the digestive tract and to tannin induced aggregation of ingested foods, rendering them less susceptible to digestion. One study estimated that due to the presence of polyphenols, digestibility of peas was limited to 50 to 80%. Polyphenol levels, and thus digestibility, are strain dependent so that the screening process noted above could serve a dual purpose. As tannins are water soluble, they should be removed during extraction of the albumin fraction.

An allergenicity associated with peas is suggested to reside within the albumin fraction of the seed proteins (Malley et al., 1975). As the low methionine product will be using the vicilin enriched fraction, allergenicity questions will hopefully be minimized.

Finally, food composition databases were searched for sources of very low methionine that would require no protein purification and a reduced amount of processing. Four foods, strawberry, chayote (a pear shape vegetable native to Central America), pea pods and tea powder were indicated to have methionine levels of 0.1% or less as a percent of total protein. Amino acid composition analysis of these four foods indicated all had methionine levels of over 0.2%. Five other whole foods, grape juice, orange juice, limes, custard apples, and instant coffee powder were indicated by the food composition databases to have methionine levels of 0.1 to 0.2% methionine. Grape juice, orange juice, and limes have too low a protein content to be commercially feasible. Instant coffee powder was tested and found to have a methionine content of greater than 0.2% of the protein. Custard apples are similarly tested for their methionine content by methods similar to those for strawberry, chayote, pea pods, and tea powder. In specific embodiments, differences between published methionine amounts and analyses reflect variances in analytical procedures and/or testing of dissimilar cultivars, or other similar reasons.

VI. Achieving Synergy Between Methionine Restriction and Other Treatments

Dietary methionine restriction may act synergistically with other cancer treatments, including methionine-associated therapies, to increase their efficacy and/or reduce their toxic side effects. There are several potential strategies for achieving synergy between methionine restriction and other treatments. One such strategy is to combine dietary methionine restriction with methionine analogues. For instance, several studies have demonstrated that methionine restriction and the methionine analogue ethionine (S-ethyl-L-homocysteine) have synergistic antitumor activity against a variety of tumors, including prostate cancer and sarcoma (Poirson-Bichat et al., 1997); Guo et al., 1996; Poirson-Bichat et al., 2000; Sasamura et al., 1999; Sasamura et al., 1998). Other methionine analogs, including selenomethionine, as well as polyamine analogs, SIBA (an analogue of S-adenosylhomocysteine), and trifluoromethylhomocysteine have also shown promise in animal studies in combination with methionine restriction (Sasamura et al., 1999; Sasamura et al., 1998; Porter et al., 1984; Porter and Sufrin, 1986; Breillout et al., 1988; Poirson-Bichat et al., 1997; Rodriguez-Vicente et al., 1998). Another approach to maximize the antitumor activity of methionine restriction is to target chemotherapy to tumors by restricting dietary methionine and then giving methionine conjugated to an anticancer drug, such as mitomycin C (Yoshida et al., 1998).

Dietary methionine restriction has also been combined with methioninase treatment to achieve maximal methionine depletion, as shown in human brain tumor xenografts in athymic mice (Kokkinakis et al., 1997).

Another potential strategy for optimizing the clinical effectiveness of methionine restriction will be to combine it with chemotherapy. Several preclinical studies have demonstrated synergy between methionine restriction and various cytotoxic chemotherapy drugs, such as 5-fluorouracil (Yoshioka et al., 1998; Goseki et al., 1991; Hoshiya et al., 1997). Methionine restriction is thought to enhance the antitumor activity of 5-fluorouracil by raising levels of 5,10-methylenetetrahydrofolate, which is the same mechanism by which leucovorm modulates 5-FU action. In Yoshida sarcoma bearing rats, methionine +/−cysteine restriction enhances the antitumor activity of 5-fluorouracil (Goseki et al., 1991). Likewise, a synergistic beneficial effect of methionine-depletion and 5-fluorouracil has been demonstrated in gastric cancer xenografts in nude mice (Hoshiya et al., 1997). Cisplatin, another commonly used chemotherapy drug, acts synergistically with methionine restriction by inhibiting methionine uptake in tumors, as demonstrated with animal breast cancer and colon cancer models (Tan et al., 1999; Hoshiya et al., 1996). Methionine restriction has also shown promise in animal studies in combination with vincristine ((Goseki et al., 1996), the alkylating agents ACNU ((Goseki and Endo, 1990) and CCNU (Kokkinakis et al., 1997), and the anthracycline doxorubicin (Goseki et al., 1992; Nagahama et al., 1998). The optimal sequence and schedule for combining methionine restriction with chemotherapy is determined empirically. One approach is to treat patients with “cyclic” methionine restriction, in much the same way as cancer patients are treated with “cycles” of chemotherapy. Allowing patients who are on methionine-restricted diets to resume normal diets briefly at regular intervals would accomplish this. In this scenario, chemotherapy would most likely be given during the brief periods of methionine repletion. Preclinical studies involving human carcinoma and sarcoma cell lines lend support to this approach. In those studies, methionine restriction was combined with doxorubicin for 10 days, and cells were then treated with methionine repletion plus vincristine (Stem and Hoffman, 1986).

Ultimately, optimization of dietary methionine restriction will depend upon clarification of the molecular mechanisms by which methionine restriction inhibits tumor growth. For instance, the fact that methionine restriction causes certain cancer cells to enter G2 cell cycle arrest (Oirson-Bichat et al,. 1997) may be exploitable therapeutically. Cancer cells that are forced to leave G2 and reenter the cell cycle prematurely following exposure to chemotherapy die much more rapidly than those remaining in G2 (Chan et al., 1999; Suganuma et al., 1999; Guo et al., 1993). “Abrogation” of G2 cell cycle arrest therefore accelerates cancer cell death. Perhaps drugs that abrogate G2 arrest can be used to increase the efficacy of dietary methionine restriction. Additional studies demonstrating that methionine restriction inhibits cyclin dependent kinases (Lu and Epner, 2001), modulates glutathione (GSH) levels (Femandez-Checa et al., 1990; Aw et al., 1984; Meredith et al., 1998; Wang et al., 1997; Djurhuus et al., 1991; Aw et al., 1986; Story and Meyn, 1999), and possibly inhibits transmethylation of DNA and other molecules (Stem and Hoffman, 1984; Tisdale, 1980; Kokkinakis et al., 1997; Chiang et al., 1996; Hoffman, 1985; Hoffman, 1984; Fiskerstrand et al., 1994; Tisdale, 1980) may also lead to the development of strategies for optimizing the effectiveness and minimizing the toxicity of methionine restriction.

VII. Cancer Therapies

Cancer therapies, known to one of skill in the art, may be used in combination with the methods and compositions of the present invention. For example, the inventors can use any of the treatments described herein on an individual with cancer in addition to any of the following cancer therapies.

A. Radiotherapeutic Agents

Radiotherapeutic agents and factors include radiation and waves that induce DNA damage: γ-irradiation, X-rays, UV-irradiation, microwaves, electronic emissions, radioisotopes, and the like. Therapy may be achieved by irradiating the localized tumor site with the above described forms of radiations. It is most likely that all of these factors effect a broad range of damage to DNA, on the precursors of DNA, the replication and repair of DNA, and the assembly and maintenance of chromosomes.

Dosage ranges for X-rays range from daily doses of 50 to 200 roentgens for prolonged periods of time (3 to 4 weeks), to single doses of 2000 to 6000 roentgens. Dosage ranges for radioisotopes vary widely, and depend on the half-life of the isotope, the strength and type of radiation emitted, and the uptake by the neoplastic cells.

B. Surgery

Surgical treatment for removal of the cancerous growth is generally a standard procedure for the treatment of tumors and cancers. This attempts to remove the entire cancerous growth. However, surgery is often combined with chemotherapy and/or radiotherapy to ensure the destruction of any remaining neoplastic or malignant cells. Thus, surgery or sham surgery may be used in the model in the context of the present invention.

C. Chemotherapeutic Agents

These can be, for example, agents that directly cross-link DNA, agents that intercalate into DNA, and agents that lead to chromosomal and mitotic aberrations by affecting nucleic acid synthesis.

Agents that directly cross-link nucleic acids, specifically DNA, are envisaged and are shown herein, to eventuate DNA damage leading to a synergistic antineoplastic combination. Agents such as cisplatin, and other DNA alkylating agents may be used.

Agents that damage DNA also include compounds that interfere with DNA replication, mitosis, and chromosomal segregation. Examples of these compounds include adriamycin (also known as doxorubicin), VP-16 (also known as etoposide), verapamil, podophyllotoxin, and the like. Widely used in clinical setting for the treatment of neoplasms these compounds are administered through bolus injections intravenously at doses ranging from 25-75 mg/m² at 21 day intervals for adriamycin, to 35-100 mg/m² for etoposide intravenously or orally.

1. Antibiotics

a. Doxorubicin

Doxorubicin hydrochloride, 5,12-Naphthacenedione, (8s-cis)-10-[(3-amino-2,3,6-trideoxy-a-L-lyxo-hexopyranosyl)oxy]-7,8,9,10-tetrahydro-6,8,11-trihydroxy-8-(hydroxyacetyl)-1-methoxy-hydrochloride (hydroxydaunorubicin hydrochloride, Adriamycin) is used in a wide antineoplastic spectrum. It binds to DNA and inhibits nucleic acid synthesis, inhibits mitosis and promotes chromosomal aberrations.

Administered alone, it is the drug of first choice for the treatment of thyroid adenoma and primary hepatocellular carcinoma. It is a component of 31 first-choice combinations for the treatment of ovarian, endometrial and breast tumors, bronchogenic oat-cell carcinoma, non-small cell lung carcinoma, gastric adenocarcinoma, retinoblastoma, neuroblastoma, mycosis fungoides, pancreatic carcinoma, prostatic carcinoma, bladder carcinoma, myeloma, diffuse histiocytic lymphoma, Wilms' tumor, Hodgkin's disease, adrenal tumors, osteogenic sarcoma soft tissue sarcoma, Ewing's sarcoma, rhabdomyosarcoma and acute lymphocytic leukemia. It is an alternative drug for the treatment of islet cell, cervical, testicular and adrenocortical cancers. It is also an immunosuppressant.

Doxorubicin is absorbed poorly and must be administered intravenously. The pharmacokinetics are multicompartmental. Distribution phases have half-lives of 12 minutes and 3.3 hr. The elimination half-life is about 30 hr. Forty to 50% is secreted into the bile. Most of the remainder is metabolized in the liver, partly to an active metabolite (doxorubicinol), but a few percent is excreted into the urine. In the presence of liver impairment, the dose should be reduced.

Appropriate doses are, intravenous, adult, 60 to 75 mg/m2 at 21-day intervals or 25 to 30 mg/m on each of 2 or 3 successive days repeated at 3- or 4-wk intervals or 20 mg/m² once a week. The lowest dose should be used in elderly patients, when there is prior bone-marrow depression caused by prior chemotherapy or neoplastic marrow invasion, or when the drug is combined with other myelopoietic suppressant drugs. The dose should be reduced by 50% if the serum bilirubin lies between 1.2 and 3 mg/dL and by 75% if above 3 mg/dL. The lifetime total dose should not exceed 550 mg/m2 in patients with normal heart function and 400 mg/m in persons having received mediastinal irradiation. Alternatively, 30 mg/m² on each of 3 consecutive days, repeated every 4 wk. Exemplary doses may be 10 mg/m², 20 mg/m², 30 mg/m², 50 mg/m², 100 mg/m², 150 mg/m², 175 mg/m², 200 mg/m², 225 mg/m², 250 mg/m², 275 mg/m², 300 mg/m², 350 mg/m², 400 mg/m², 425 mg/m², 450 mg/m², 475 mg/m², 500 mg/m². Of course, all of these dosages are exemplary, and any dosage in-between these points is also expected to be of use in the invention.

b. Daunorubicin

Daunorubicin hydrochloride, 5,12-Naphthacenedione, (8S-cis)-8-acetyl-10-[(3-amino-2,3,6-trideoxy-a-L-lyxo-hexanopyranosyl)oxy]-7,8,9,10-tetrahydro-6,8,11-trihydroxy-10-methoxy-, hydrochloride; also termed cerubidine and available from Wyeth. Daunorubicin intercalates into DNA, blocks DAN-directed RNA polymerase and inhibits DNA synthesis. It can prevent cell division in doses that do not interfere with nucleic acid synthesis.

In combination with other drugs it is included in the first-choice chemotherapy of acute myelocytic leukemia in adults (for induction of remission), acute lymphocytic leukemia and the acute phase of chronic myclocytic leukemia. Oral absorption is poor, and it must be given intravenously. The half-life of distribution is 45 minutes and of elimination, about 19 hr. The half-life of its active metabolite, daunorubicinol, is about 27 hr. Daunorubicin is metabolized mostly in the liver and also secreted into the bile (ca 40%). Dosage must be reduced in liver or renal insufficiencies.

Suitable doses are (base equivalent), intravenous adult, younger than 60 yr. 45 mg/m²/day (30 mg/m² for patients older than 60 yr.) for 1, 2 or 3 days every 3 or 4 wk or 0.8 mg/kg/day for 3 to 6 days every 3 or 4 wk; no more than 550 mg/m² should be given in a lifetime, except only 450 mg/m² if there has been chest irradiation; children, 25 mg/m² once a week unless the age is less than 2 yr. or the body surface less than 0.5 nm, in which case the weight-based adult schedule is used. It is available in injectable dosage forms (base equivalent) 20 mg (as the base equivalent to 21.4 mg of the hydrochloride). Exemplary doses may be 10 mg/m², 20 mg/m², 30 mg/m², 50 mg/m², 100 mg/m², 150 mg/m², 175 mg/m², 200 mg/m², 225 mg/m², 250 mg/m², 275 mg/m², 300 mg/m², 350 mg/m², 400 mg/m², 425 mg/m², 450 mg/m², 475 mg/m², 500 mg/m². Of course, all of these dosages are exemplary, and any dosage in-between these points is also expected to be of use in the invention.

C. Mitomycin

Mitomycin (also known as mutamycin and/or mitomycin-C) is an antibiotic isolated from the broth of Streptomyces caespitosus which has been shown to have antitumor activity. The compound is heat stable, has a high melting point, and is freely soluble in organic solvents.

Mitomycin selectively inhibits the synthesis of deoxyribonucleic acid (DNA). The guanine and cytosine content correlates with the degree of mitomycin-induced cross-linking. At high concentrations of the drug, cellular RNA and protein synthesis are also suppressed.

In humans, mitomycin is rapidly cleared from the serum after intravenous administration. Time required to reduce the serum concentration by 50% after a 30 mg. bolus injection is 17 minutes. After injection of 30 mg., 20 mg., or 10 mg. I.V., the maximal serum concentrations were 2.4 mg./mL, 1.7 mg./mL, and 0.52 mg./mL, respectively. Clearance is effected primarily by metabolism in the liver, but metabolism occurs in other tissues as well. The rate of clearance is inversely proportional to the maximal serum concentration because, it is thought, of saturation of the degradative pathways.

Approximately 10% of a dose of mitomycin is excreted unchanged in the urine. Since metabolic pathways are saturated at relatively low doses, the percent of a dose excreted in urine increases with increasing dose. In children, excretion of intravenously administered mitomycin is similar.

d. Actinomycin D

Actinomycin D (Dactinomycin) [50-76-0]; C₆₂H₈₆N₁₂O₁₆ (1255.43) is an antineoplastic drug that inhibits DNA-dependent RNA polymerase. It is a component of first-choice combinations for treatment of choriocarcinoma, embryonal rhabdomyosarcoma, testicular tumor and Wilms' tumor. Tumors which fail to respond to systemic treatment sometimes respond to local perfusion. Dactinomycin potentiates radiotherapy. It is a secondary (efferent) immunosuppressive.

Actinomycin D is used in combination with primary surgery, radiotherapy, and other drugs, particularly vincristine and cyclophosphamide. Antineoplastic activity has also been noted in Ewing's tumor, Kaposi's sarcoma, and soft-tissue sarcomas. Dactinomycin can be effective in women with advanced cases of choriocarcinoma. It also produces consistent responses in combination with chlorambucil and methotrexate in patients with metastatic testicular carcinomas. A response may sometimes be observed in patients with Hodgkin's disease and non-Hodgkin's lymphomas. Dactinomycin has also been used to inhibit immunological responses, particularly the rejection of renal transplants.

Half of the dose is excreted intact into the bile and 10% into the urine; the half-life is about 36 hr. The drug does not pass the blood-brain barrier. Actinomycin D is supplied as a lyophilized powder (0/5 mg in each vial). The usual daily dose is 10 to 15 mg/kg; this is given intravenously for 5 days; if no manifestations of toxicity are encountered, additional courses may be given at intervals of 3 to 4 weeks. Daily injections of 100 to 400 mg have been given to children for 10 to 14 days; in other regimens, 3 to 6 mg/kg, for a total of 125 mg/kg, and weekly maintenance doses of 7.5 mg/kg have been used. Although it is safer to administer the drug into the tubing of an intravenous infusion, direct intravenous injections have been given, with the precaution of discarding the needle used to withdraw the drug from the vial in order to avoid subcutaneous reaction. Exemplary doses may be 100 mg/m², 150 mg/m², 175 mg/m², 200 mg/m², 225 mg/m², 250 mg/m², 275 mg/m², 300 mg/m², 350 mg/m², 400 mg/m², 425 mg/m², 450 mg/m², 475 mg/m², 500 mg/m². Of course, all of these dosages are exemplary, and any dosage in-between these points is also expected to be of use in the invention.

e. Bleomycin

Bleomycin is a mixture of cytotoxic glycopeptide antibiotics isolated from a strain of Streptomyces verticillus. It is freely soluble in water.

Although the exact mechanism of action of bleomycin is unknown, available evidence would seem to indicate that the main mode of action is the inhibition of DNA synthesis with some evidence of lesser inhibition of RNA and protein synthesis.

In mice, high concentrations of bleomycin are found in the skin, lungs, kidneys, peritoneum, and lymphatics. Tumor cells of the skin and lungs have been found to have high concentrations of bleomycin in contrast to the low concentrations found in hematopoietic tissue. The low concentrations of bleomycin found in bone marrow may be related to high levels of bleomycin degradative enzymes found in that tissue.

In patients with a creatinine clearance of >35 mL per minute, the serum or plasma terminal elimination half-life of bleomycin is approximately 115 minutes. In patients with a creatinine clearance of <35 mL per minute, the plasma or serum terminal elimination half-life increases exponentially as the creatinine clearance decreases. In humans, 60% to 70% of an administered dose is recovered in the urine as active bleomycin.

Bleomycin should be considered a palliative treatment. It has been shown to be useful in the management of the following neoplasms either as a single agent or in proven combinations with other approved chemotherapeutic agents in squamous cell carcinoma such as head and neck (including mouth, tongue, tonsil, nasopharynx, oropharynx, sinus, palate, lip, buccal mucosa, gingiva, epiglottis, larynx), skin, penis, cervix, and vulva. It has also been used in the treatment of lymphomas and testicular carcinoma.

Because of the possibility of an anaphylactoid reaction, lymphoma patients should be treated with two units or less for the first two doses. If no acute reaction occurs, then the regular dosage schedule may be followed.

Improvement of Hodgkin's Disease and testicular tumors is prompt and noted within 2 weeks. If no improvement is seen by this time, improvement is unlikely. Squamous cell cancers respond more slowly, sometimes requiring as long as 3 weeks before any improvement is noted.

Bleomycin may be given by the intramuscular, intravenous, or subcutaneous routes.

2. Miscellaneous Agents

a. Cisplatin

Cisplatin has been widely used to treat cancers such as metastatic testicular or ovarian carcinoma, advanced bladder cancer, head or neck cancer, cervical cancer, lung cancer or other tumors. Cisplatin can be used alone or in combination with other agents, with efficacious doses used in clinical applications of 15-20 mg/m² for 5 days every three weeks for a total of three courses. Exemplary doses may be 0.50 mg/m², 1.0 mg/m², 1.50 mg/m², 1.75 mg/m², 2.0 mg/m², 3.0 mg/m², 4.0 mg/m², 5.0 mg/m², 10 mg//m². Of course, all of these dosages are exemplary, and any dosage in-between these points is also expected to be of use in the invention.

Cisplatin is not absorbed orally and must therefore be delivered via injection intravenously, subcutaneously, intratumorally or intraperitoneally.

In certain aspects of the current invention cisplatin is used in combination with emodin or emodin-like compounds in the treatment of non-small cell lung carcinoma. It is clear, however, that the combination of cisplatin and emodin and or emodin-like compounds could be used for the treatment of any other neu-mediated cancer.

b. VP16

VP16 is also know as etoposide and is used primarily for treatment of testicular tumors, in combination with bleomycin and cisplatin, and in combination with cisplatin for small-cell carcinoma of the lung. It is also active against non-Hodgkin's lymphomas, acute nonlymphocytic leukemia, carcinoma of the breast, and Kaposi's sarcoma associated with acquired immunodeficiency syndrome (AIDS).

VP16 is available as a solution (20 mg/ml) for intravenous administration and as 50-mg, liquid-filled capsules for oral use. For small-cell carcinoma of the lung, the intravenous dose (in combination therapy) is can be as much as 100 mg/m² or as little as 2 mg/m², routinely 35 mg/m², daily for 4 days, to 50 mg/m², daily for 5 days have also been used. When given orally, the dose should be doubled. Hence the doses for small cell lung carcinoma may be as high as 200-250 mg/m². The intravenous dose for testicular cancer (in combination therapy) is 50 to 100 mg/m² daily for 5 days, or 100 mg/m² on alternate days, for three doses. Cycles of therapy are usually repeated every 3 to 4 weeks. The drug should be administered slowly during a 30- to 60-minute infusion in order to avoid hypotension and bronchospasm, which are probably due to the solvents used in the formulation.

C. Tumor Necrosis Factor

Tumor Necrosis Factor [TNF; Cachectin] is a glycoprotein that kills some kinds of cancer cells, activates cytokine production, activates macrophages and endothelial cells, promotes the production of collagen and collagenases, is an inflammatory mediator and also a mediator of septic shock, and promotes catabolism, fever and sleep. Some infectious agents cause tumor regression through the stimulation of TNF production. TNF can be quite toxic when used alone in effective doses, so that the optimal regimens probably will use it in lower doses in combination with other drugs. Its immunosuppressive actions are potentiated by gamma-interferon, so that the combination potentially is dangerous. A hybrid of TNF and interferon-α also has been found to possess anti-cancer activity.

3. Plant Alkaloids

a. Taxol

Taxol is an experimental antimitotic agent, isolated from the bark of the ash tree, Taxus brevifolia. It binds to tubulin (at a site distinct from that used by the vinca alkaloids) and promotes the assembly of microtubules. Taxol is currently being evaluated clinically; it has activity against malignant melanoma and carcinoma of the ovary. Maximal doses are 30 mg/m² per day for 5 days or 210 to 250 mg/m² given once every 3 weeks. Of course, all of these dosages are exemplary, and any dosage in-between these points is also expected to be of use in the invention.

b. Vincristine

Vincristine blocks mitosis and produces metaphase arrest. It seems likely that most of the biological activities of this drug can be explained by its ability to bind specifically to tubulin and to block the ability of protein to polymerize into microtubules. Through disruption of the microtubules of the mitotic apparatus, cell division is arrested in metaphase. The inability to segregate chromosomes correctly during mitosis presumably leads to phasic pattern of clearance from the plasma; the terminal half-life is about 24 hours. The drug is metabolized in the liver, but no biologically active derivatives have been identified. Doses should be reduced in patients with hepatic dysfunction. At least a 50% reduction in dosage is indicated if the concentration of bilirubin in plasma is greater than 3 mg/dl (about 50 mM).

Vincristine sulfate is available as a solution (1 mg/ml) for intravenous injection. Vincristine used together with corticosteroids is presently the treatment of choice to induce remissions in childhood leukemia; the optimal dosages for these drugs appear to be vincristine, intravenously, 2 mg/m² of body-surface area, weekly, and prednisolone, orally, 40 mg/m², daily. Adult patients with Hodgkin's disease or non-Hodgkin's lymphomas usually receive vincristine as a part of a complex protocol. When used in the MOPP regimen, the recommended dose of vincristine is 1.4 mg/m². High doses of vincristine seem to be tolerated better by children with leukemia than by adults, who may experience sever neurological toxicity. Administration of the drug more frequently than every 7 days or at higher doses seems to increase the toxic manifestations without proportional improvement in the response rate. Precautions should also be used to avoid extravasation during intravenous administration of vincristine. Vincristine (and vinblastine) can be infused into the arterial blood supply of tumors in doses several times larger than those that can be administered intravenously with comparable toxicity.

Vincristine has been effective in Hodgkin's disease and other lymphomas. Although it appears to be somewhat less beneficial than vinblastine when used alone in Hodgkin's disease, when used with mechlorethamine, prednisolone, and procarbazine (the so-called MOPP regimen), it is the preferred treatment for the advanced stages (III and IV) of this disease. In non-Hodgkin's lymphomas, vincristine is an important agent, particularly when used with cyclophosphamide, bleomycin, doxorubicin, and prednisolone. Vincristine is more useful than vinblastine in lymphocytic leukemia. Beneficial response have been reported in patients with a variety of other neoplasms, particularly Wilms' tumor, neuroblastoma, brain tumors, rhabdomyosarcoma, and carcinomas of the breast, bladder, and the male and female reproductive systems.

Doses of vincristine for use will be determined by the clinician according to the individual patients need. 0.01 to 0.03 mg/kg or 0.4 to 1.4 mg/m² can be administered or 1.5 to 2 mg/m² can also be administered. Alternatively 0.02 mg/m², 0.05 mg/m², 0.06 mg/m², 0.07 mg/m², 0.08 mg/m², 0.1 mg/m², 0.12 mg/m², 0.14 mg/m², 0.15 mg/m², 0.2 mg/m², 0.25 mg/m² can be given as a constant intravenous infusion. Of course, all of these dosages are exemplary, and any dosage in-between these points is also expected to be of use in the invention.

C. Vinblastine

When cells are incubated with vinblastine, dissolution of the microtubules occurs. Unpredictable absorption has been reported after oral administration of vinblastine or vincristine. At the usual clinical doses the peak concentration of each drug in plasma is approximately 0.4 mM. Vinblastine and vincristine bind to plasma proteins. They are extensively concentrated in platelets and to a lesser extent in leukocytes and erythrocytes.

After intravenous injection, vinblastine has a multiphasic pattern of clearance from the plasma; after distribution, drug disappears from plasma with half-lives of approximately 1 and 20 hours.

Vinblastine is metabolized in the liver to biologically activate derivative desacetylvinblastine. Approximately 15% of an administered dose is detected intact in the urine, and about 10% is recovered in the feces after biliary excretion. Doses should be reduced in patients with hepatic dysfunction. At least a 50% reduction in dosage is indicated if the concentration of bilirubin in plasma is greater than 3 mg/dl (about 50 mM).

Vinblastine sulfate is available in preparations for injection. The drug is given intravenously; special precautions must be taken against subcutaneous extravasation, since this may cause painful irritation and ulceration. The drug should not be injected into an extremity with impaired circulation. After a single dose of 0.3 mg/kg of body weight, myelosuppression reaches its maximum in 7 to 10 days. If a moderate level of leukopenia (approximately 3000 cells/mm³) is not attained, the weekly dose may be increased gradually by increments of 0.05 mg/kg of body weight. In regimens designed to cure testicular cancer, vinblastine is used in doses of 0.3 mg/kg every 3 weeks irrespective of blood cell counts or toxicity.

The most important clinical use of vinblastine is with bleomycin and cisplatin in the curative therapy of metastatic testicular tumors. Beneficial responses have been reported in various lymphomas, particularly Hodgkin's disease, where significant improvement may be noted in 50 to 90% of cases. The effectiveness of vinblastine in a high proportion of lymphomas is not diminished when the disease is refractory to alkylating agents. It is also active in Kaposi's sarcoma, neuroblastoma, and Letterer-Siwe disease (histiocytosis X), as well as in carcinoma of the breast and choriocarcinoma in women.

Doses of vinblastine for use will be determined by the clinician according to the individual patients need. 0.1 to 0.3 mg/kg can be administered or 1.5 to 2 mg/m² can also be administered. Alternatively, 0.1 mg/m², 0.12 mg/m², 0.14 mg/m², 0.15 mg/m², 0.2 mg/m², 0.25 mg/m², 0.5 mg/m², 1.0 mg/m², 1.2 mg/m², 1.4 mg/m², 1.5 mg/m², 2.0 mg/m², 2.5 mg/m², 5.0 mg/m², 6 mg/m², 8 mg/m², 9 mg/m², 10 mg/m², 20 mg/m², can be given. Of course, all of these dosages are exemplary, and any dosage in-between these points is also expected to be of use in the invention.

4. Alkylating Agents

a. Carmustine

Carmustine (sterile carmustine) is one of the nitrosoureas used in the treatment of certain neoplastic diseases. It is 1,3bis (2-chloroethyl)-1-nitrosourea. It is lyophilized pale yellow flakes or congealed mass with a molecular weight of 214.06. It is highly soluble in alcohol and lipids, and poorly soluble in water. Carmustine is administered by intravenous infusion after reconstitution as recommended. Sterile carmustine is commonly available in 100 mg single dose vials of lyophilized material.

Although it is generally agreed that carmustine alkylates DNA and RNA, it is not cross resistant with other alkylators. As with other nitrosoureas, it may also inhibit several key enzymatic processes by carbamoylation of amino acids in proteins.

Carmustine is indicated as palliative therapy as a single agent or in established combination therapy with other approved chemotherapeutic agents in brain tumors such as glioblastoma, brainstem glioma, medullobladyoma, astrocytoma, ependymoma, and metastatic brain tumors. Also it has been used in combination with prednisolone to treat multiple myeloma. Carmustine has proved useful, in the treatment of Hodgkin's Disease and in non-Hodgkin's lymphomas, as secondary therapy in combination with other approved drugs in patients who relapse while being treated with primary therapy, or who fail to respond to primary therapy.

The recommended dose of carmustine as a single agent in previously untreated patients is 150 to 200 mg/m2 intravenously every 6 weeks. This may be given as a single dose or divided into daily injections such as 75 to 100 mg/m² on 2 successive days. When carmustine is used in combination with other myelosuppressive drugs or in patients in whom bone marrow reserve is depleted, the doses should be adjusted accordingly. Doses subsequent to the initial dose should be adjusted according to the hematologic response of the patient to the preceding dose. It is of course understood that other doses may be used in the present invention for example 10 mg/m², 20 mg/m², 30 mg/m² 40 mg/m² 50 mg/m² 60 mg/m² 70 mg/m² 80 mg/m² 90 mg/m² 100 mg/m². The skilled artisan is directed to, “Remington's Pharmaceutical Sciences” 15th Edition, chapter 61. Some variation in dosage will necessarily occur depending on the condition of the subject being treated. The person responsible for administration will, in any event, determine the appropriate dose for the individual subject

b. Melphalan

Melphalan also known as alkeran, L-phenylalanine mustard, phenylalanine mustard, L-PAM, or L-sarcolysin, is a phenylalanine derivative of nitrogen mustard. Melphalan is a bifunctional alkylating agent which is active against selective human neoplastic diseases. It is known chemically as 4-[bis(2-chloroethyl)amino]-L-phenylalanine.

Melphalan is the active L-isomer of the compound and was first synthesized in 1953 by Bergel and Stock; the D-isomer, known as medphalan, is less active against certain animal tumors, and the dose needed to produce effects on chromosomes is larger than that required with the L-isomer. The racemic (DL-) form is known as merphalan or sarcolysin. Melphalan is insoluble in water and has a pKa₁ of ˜2.1. Melphalan is available in tablet form for oral administration and has been used to treat multiple myeloma.

Available evidence suggests that about one third to one half of the patients with multiple myeloma show a favorable response to oral administration of the drug.

Melphalan has been used in the treatment of epithelial ovarian carcinoma. One commonly employed regimen for the treatment of ovarian carcinoma has been to administer melphalan at a dose of 0.2 mg/kg daily for five days as a single course. Courses are repeated every four to five weeks depending upon hematologic tolerance (Smith and Rutledge, 1975; Young et al., 1978). Alternatively the dose of melphalan used could be as low as 0.05 mg/kg/day or as high as 3 mg/kg/day or any dose in between these doses or above these doses. Some variation in dosage will necessarily occur depending on the condition of the subject being treated. The person responsible for administration will, in any event, determine the appropriate dose for the individual subject.

C. Cyclophosphamide

Cyclophosphamide is 2H-1,3,2-Oxazaphosphorin-2-amine, N,N-bis(2-chloroethyl)tetrahydro-, 2-oxide, monohydrate; termed Cytoxan available from Mead Johnson; and Neosar available from Adria. Cyclophosphamide is prepared by condensing 3-amino-1-propanol with N,N-bis(2-chlorethyl) phosphoramidic dichloride [(ClCH₂CH₂)₂N—POCl₂] in dioxane solution under the catalytic influence of triethylamine. The condensation is double, involving both the hydroxyl and the amino groups, thus effecting the cyclization.

Unlike other β-chloroethylamino alkylators, it does not cyclize readily to the active ethyleneimonium form until activated by hepatic enzymes. Thus, the substance is stable in the gastrointestinal tract, tolerated well and effective by the oral and parental routes and does not cause local vesication, necrosis, phlebitis or even pain.

Suitable doses for adults include, orally, 1 to 5 mg/kg/day (usually in combination), depending upon gastrointestinal tolerance; or 1 to 2 mg/kg/day; intravenously, initially 40 to 50 mg/kg in divided doses over a period of 2 to 5 days or 10 to 15 mg/kg every 7 to 10 days or 3 to 5 mg/kg twice a week or 1.5 to 3 mg/kg/day. A dose 250 mg/kg/day may be administered as an antineoplastic. Because of gastrointestinal adverse effects, the intravenous route is preferred for loading. During maintenance, a leukocyte count of 3000 to 4000/mm³ usually is desired. The drug also sometimes is administered intramuscularly, by infiltration or into body cavities. It is available in dosage forms for injection of 100, 200 and 500 mg, and tablets of 25 and 50 mg the skilled artisan is referred to “Remington's Pharmaceutical Sciences” 15th Edition, chapter 61, incorporate herein as a reference, for details on doses for administration.

d. Chlorambucil

Chlorambucil (also known as leukeran) was first synthesized by Everett et al. (1953). It is a bifunctional alkylating agent of the nitrogen mustard type that has been found active against selected human neoplastic diseases. Chlorambucil is known chemically as 4-[bis(2-chlorethyl)amino] benzenebutanoic acid.

Chlorambucil is available in tablet form for oral administration. It is rapidly and completely absorbed from the gastrointestinal tract. After single oral doses of 0.6-1.2 mg/kg, peak plasma chlorambucil levels are reached within one hour and the terminal half-life of the parent drug is estimated at 1.5 hours. 0.1 to 0.2 mg/kg/day or 3 to 6 mg/m²/day or alternatively 0.4 mg/kg may be used for antineoplastic treatment. Treatment regimes are well know to those of skill in the art and can be found in the “Physicians Desk Reference” and in “Remingtons Pharmaceutical Sciences” referenced herein.

Chlorambucil is indicated in the treatment of chronic lymphatic (lymphocytic) leukemia, malignant lymphomas including lymphosarcoma, giant follicular lymphoma and Hodgkin's disease. It is not curative in any of these disorders but may produce clinically useful palliation.

e. Busulfan

Busulfan (also known as myleran) is a bifunctional alkylating agent. Busulfan is known chemically as 1,4-butanediol dimethanesulfonate.

Busulfan is not a structural analog of the nitrogen mustards. Busulfan is available in tablet form for oral administration. Each scored tablet contains 2 mg busulfan and the inactive ingredients magnesium stearate and sodium chloride.

Busulfan is indicated for the palliative treatment of chronic myelogenous (myeloid, myelocytic, granulocytic) leukemia. Although not curative, busulfan reduces the total granulocyte mass, relieves symptoms of the disease, and improves the clinical state of the patient. Approximately 90% of adults with previously untreated chronic myelogenous leukemia will obtain hematologic remission with regression or stabilization of organomegaly following the use of busulfan. It has been shown to be superior to splenic irradiation with respect to survival times and maintenance of hemoglobin levels, and to be equivalent to irradiation at controlling splenomegaly.

f. Lomustine

Lomustine is one of the nitrosoureas used in the treatment of certain neoplastic diseases. It is 1-(2-chloro-ethyl)-3-cyclohexyl-1 nitrosourea. It is a yellow powder with the empirical formula of C₉H₁₆ClN₃O₂ and a molecular weight of 233.71. Lomustine is soluble in 10% ethanol (0.05 mg per mL) and in absolute alcohol (70 mg per mL). Lomustine is relatively insoluble in water (<0.05 mg per mL). It is relatively unionized at a physiological pH. Inactive ingredients in lomustine capsules are: magnesium stearate and mannitol.

Although it is generally agreed that lomustine alkylates DNA and RNA, it is not cross resistant with other alkylators. As with other nitrosoureas, it may also inhibit several key enzymatic processes by carbamoylation of amino acids in proteins.

Lomustine may be given orally. Following oral administration of radioactive lomustine at doses ranging from 30 mg/m² to 100 mg/m², about half of the radioactivity given was excreted in the form of degradation products within 24 hours.

The serum half-life of the metabolites ranges from 16 hours to 2 days. Tissue levels are comparable to plasma levels at 15 minutes after intravenous administration.

Lomustine has been shown to be useful as a single agent in addition to other treatment modalities, or in established combination therapy with other approved chemotherapeutic agents in both primary and metastatic brain tumors, in patients who have already received appropriate surgical and/or radiotherapeutic procedures. It has also proved effective in secondary therapy against Hodgkin's Disease in combination with other approved drugs in patients who relapse while being treated with primary therapy, or who fail to respond to primary therapy.

The recommended dose of lomustine in adults and children as a single agent in previously untreated patients is 130 mg/m² as a single oral dose every 6 weeks. In individuals with compromised bone marrow function, the dose should be reduced to 100 mg/m² every 6 weeks. When lomustine is used in combination with other myelosuppressive drugs, the doses should be adjusted accordingly. It is understood that other doses may be used for example, 20 mg/m² 30 mg/m², 40 mg/m², 50 mg/m², 60 mg/m², 70 mg/m², 80 mg/m², 90 mg/m², 100 mg/m², 120 mg/m² or any doses between these figures as determined by the clinician to be necessary for the individual being treated.

Additional chemotherapeutic agents include ifosphamide, carboplatin, antimetabolies, such as 5-fluorouracil (5FU), 6 MP, methotrexate), taxotere, mitoxantrone, capecitabine, topotecan, and vinorelbine.

VIII. Pharmaceutical Preparations

In a specific embodiment, the methionine-level reducing agents of the present invention are pharmaceutical preparations. Pharmaceutical compositions of the present invention comprise a therapeutically effective amount of a methionine-level reducing agent dissolved or dispersed in a pharmaceutically acceptable carrier. For a diet of the present invention, a therapeutically effective amount of methionine may be from 0-2 mg/kg/day in a methionine-restricted diet and from 10 to 30 mg/kg/day in a methionine-replete diet. The phrases “pharmaceutical or pharmacologically acceptable” refers to molecular entities and compositions that do not produce an adverse, allergic or other untoward reaction when administered to an animal, such as, for example, a human, as appropriate. The preparation of a pharmaceutical composition that contains a therapeutically effective amount of methionine or additional active ingredient will be known to those of skill in the art in light of the present disclosure, as exemplified by Remington's Pharmaceutical Sciences, 18th Ed. Mack Printing Company, 1990, incorporated herein by reference. Moreover, for animal (e.g., human) administration, it will be understood that preparations should meet sterility, pyrogenicity, general safety and purity standards as required by FDA Office of Biological Standards. In a specific embodiment, the diet composition of the present invention further comprises a chemotherapeutic agent for the treatment of the cancer, such as a drug or a gene therapy composition.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the diet of the present invention, its use in the therapeutic or pharmaceutical compositions is contemplated.

The methionine-level reducing agent may comprise different types of carriers depending on whether it is to be administered in solid or liquid form, preferably enterally. However, in embodiments wherein a chemotherapeutic agent is administered in addition to the diet, administrations can be intravenously, intradermally, intraarterially, intraperitoneally, intralesionally, intracranially, intraarticularly, intraprostaticaly, intrapleurally, intratracheally, intranasally, intravitreally, intravaginally, intrarectally, topically, intratumorally, intramuscularly, subcutaneously, subconjunctival, intravesicularlly, mucosally, intrapericardially, intraumbilically, intraocularally, orally, topically, locally, inhalation (e.g. aerosol inhalation), injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art (see, for example, Remington's Pharmaceutical Sciences, 18th Ed. Mack Printing Company, 1990, incorporated herein by reference).

The actual dosage amount of a composition of the present invention administered to an animal patient can be determined by physical and physiological factors such as body weight, severity of condition, the type of disease being treated, previous or concurrent therapeutic interventions, idiopathy of the patient and on the route of administration. The practitioner responsible for administration will, in any event, determine or otherwise be cognizant of the concentration of methionine in a composition and appropriate dose(s) for the individual subject.

In any case, the composition may comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

In embodiments where the composition is in a liquid form, a carrier can be a solvent or dispersion medium comprising but not limited to, water, ethanol, polyol (e.g., glycerol, propylene glycol, liquid polyethylene glycol, etc), lipids (e.g., triglycerides, vegetable oils, liposomes) and combinations thereof. The proper fluidity can be maintained, for example, by the use of a coating, such as lecithin; by the maintenance of the required particle size by dispersion in carriers such as, for example liquid polyol or lipids; by the use of surfactants such as, for example hydroxypropylcellulose; or combinations thereof such methods. In many cases, it will be preferable to include isotonic agents, such as, for example, sugars, sodium chloride or combinations thereof.

In certain embodiments the methionine-level reducing agent is prepared for administration by such routes as oral ingestion. In these embodiments, the solid composition may comprise, for example, solutions, suspensions, emulsions, tablets, pills, capsules (e.g., hard or soft shelled gelatin capsules), sustained release formulations, buccal compositions, troches, elixirs, suspensions, syrups, wafers, or combinations thereof. Examples of carriers for oral administration comprise inert diluents, assimilable edible carriers or combinations thereof. In other aspects of the invention, the oral composition may be prepared as a syrup or elixir. A syrup or elixir, and may comprise, for example, at least one active agent, a sweetening agent, a preservative, a flavoring agent, a dye, a preservative, or combinations thereof. In one embodiment, a solid product would most likely be a dietary powder to be reconstituted into a liquid suspension at the point of consumption (enteral).

In certain preferred embodiments an oral composition may comprise one or more binders, excipients, disintegration agents, lubricants, flavoring agents, and combinations thereof. In certain embodiments, a composition may comprise one or more of the following: a binder, such as, for example, gum tragacanth, acacia, cornstarch, gelatin or combinations thereof; an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc.; or combinations thereof the foregoing. When the dosage unit form is a capsule, it may contain, in addition to materials of the above type, carriers such as a liquid carrier. Various other materials may be present as coatings or to otherwise modify the physical form of the dosage unit. For instance, tablets, pills, or capsules may be coated with shellac, sugar or both.

The composition must be stable under the conditions of manufacture and storage, and preserved against the contaminating action of microorganisms, such as bacteria and fungi. It will be appreciated that endotoxin contamination should be kept minimally at a safe level, for example, less that 0.5 ng/mg protein.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Materials and Methods-Patients

Twelve patients at the Houston VA Medical Center with histological or cytological proof of metastatic cancer who were either refractory to standard therapy or had a disease for which no standard therapy existed were entered between March 1999 and November 2000. Two patients were taken off the study after only two weeks due to rapidly progressive cancer and rapid decline in performance status. Two other patients elected to discontinue the study after less than a month even though they felt well and remained clinically stable. Data from the remaining eight patients were analyzed. Characteristics of the patients are shown in Table 6.

TABLE 6
Patient characteristics.

| | Zubrod Performance | Weeks on |
|---|---|---|
| Diagnosis | Status | Trial |
| Renal Cell Carcinoma | 0 | 39 |
| Carcinoid | 1 | 14 |
| Sarcoma | 2 | 8 |
| Pancreatic Adenocarcinoma | 2 | 8 |
| Renal Cell Carcinoma | 2 | 16 |
| Prostate Adenocarcinoma | 2 | 12 |
| Follicular lymphoma | 0 | 16 |
| Renal Cell Carcinoma | 1 | 16 |

They were on the experimental diet for an average of 17.3 weeks (range 8-39). All patients had progressive disease at the time of enrollment. Progressive disease was defined as progressive growth of bidimensionally measurable soft tissue disease or new bone scan lesions. The patient with prostate cancer had a rising prostate specific antigen (PSA) on two successive determinations at least one month apart and failed an adequate trial of hormonal therapy, which consisted of luteinizing hormone releasing hormone (LHRH) agonist plus antiandrogen with serum testosterone <50 ng/dL. The patient with prostate cancer remained on the LHRH agonist throughout participation and was taken off antiandrogen more than two months prior to enrollment. Eligibility requirements included an estimated life expectancy of at least 12 weeks, Zubrod performance status of 0-2, and age >18. Patients received no chemotherapy or radiation therapy for at least six weeks before study entry. Baseline laboratory parameters included a neutrophil count >1500/mm³, platelet count >100,000/mm3, hemoglobin >9 g/dL, bilirubin <1.5 mg/dL, serum aspartate aminotransferase and serum alanine aminotransferase <2.5×normal, and serum creatinine level of ≦2.0 mg/dL. Patients with a history of significant cardiac disease, metabolic disorder, infection, or brain metastases were excluded. Only the patient with pancreatic adenocarcinoma experienced weight loss, totaling 15 kg, during the year prior to enrollment.

### Example 2

### Materials and Methods-Patient Monitoring

Before therapy, all patients had a complete history and physical examination. Pretreatment laboratory evaluation parameters included plasma amino acid profile, total plasma homocysteine, complete blood count, platelet count, sodium, potassium, chloride, carbon dioxide, blood urea nitrogen, creatinine, glucose, calcium, protein, albumin, phosphorus, uric acid, serum aspartate aminotransferase, serum alanine aminotransferase. bilirubin, lactate dehydrogenase, alkaline phosphatase, lipoprotein panel, prothrombin time (PT), and partial thromboplastin time (PTT). Plasma amino acid determinations were performed using a Dionix HPLC with post column ninhydrin detection. Total plasma homocysteine was determined by electrospray tandem mass spectroscopy as previously described (Magera et al., 1999). Plasma amino acid profile and total plasma homocysteine levels were measured twice per week for the first two weeks and every other week thereafter. The history, physical exam, serum chemistries, and blood counts were repeated every two weeks. Calcium and lipoprotein panel were repeated every eight weeks. Participants were monitored for side effects according to common toxicity criteria of the National Cancer Institute.

### Example 3

### Materials and Methods-Development of the Methionine-Restricted Diet

The protocol for implementing the dietary methionine restriction was modified over the course of this study in order to develop a dietary pattern that could best be used by free-living cancer patients. All subjects were placed on the “old Hominex®-2” Amino Acid-Modified Medical Food (Ross Products Division, Abbott Laboratories, Columbus, Ohio), which is approved for treatment of patients with homocystinuria (see Tables 1, 2, and 3).

The quantity of Hominex®-2 consumed daily by each subject was calculated to provide 0.6-0.8 gm protein per kg body weight. Hominex®-2 dose and energy intake were maintained at baseline levels throughout participation rather than being reduced as patients lost weight. Hominex®-2 served as the primary dietary protein source for all subjects.

The first four participants were placed on a modular diet designed to provide 2 mg methionine/kg/day and approximately 25 kcals/kg/day. The diet consisted of low-protein cereals, grains and breads; fruits; vegetables; margarines and oils; and simple carbohydrates (sodas, hard candies, sugars, and drink mixes). During this period, Hominex®-2 was mixed with water and/or low protein broth in an effort to maximize the amount of regular food that subjects could consume. However, over the course of treatment of these initial subjects, it became apparent that all were struggling to consume sufficient quantities of food to meet energy needs. The initial subjects consistently complained of early satiety and lack of appetite. Commercially available low-protein foods proved difficult for subjects to obtain and were not palatable.

The protocol was therefore modified so that Hominex®-2 served as the primary source of both protein and energy. Hominex®-2 mixtures were modified using powdered, citrus-flavored drink mixes, protein-free bouillon, tomato juice, sucrose, and canola oil. These Hominex®-2 “shakes” were formulated based on the energy requirements of each subject. By consuming 4-5 shakes per day, subjects met 100% of their protein requirements (0.8 gm protein/kg) and approximately 75% of their energy requirements. Regular food was then used to provide up to 2 mg methionine/kg/day as well as the remaining energy needs. Following the reformulation of the beverage, subjects were able to consume up to 35 kcals/kg/day.

Methionine is present in most foods as an integral component of dietary protein. Following the reformulation of Hominex®-2 shakes, dietary methionine exchange lists were developed which allowed subjects to select and consume a variety of foods up to their targeted dietary methionine level. Subjects could choose small portions of dietary starches (cereals, potatoes, breads, crackers, canned soups, cookies, etc.) and ample portions of fruits and vegetables. Use of protein-free beverages, candies, ices, margarines, and cooking oils served to boost energy intake into target ranges. Patients were counseled not to eat any foods containing animal protein, which is rich in methionine.

### Example 4

### Materials and Methods-Nutritional Intervention and Monitoring

Subjects were seen by the study dietitian prior to initiating the study. At this session, the dietary regimen was reviewed and subjects were instructed on methods for keeping food records. Subjects kept a food record for one week prior to initiating the study and weekly thereafter. Upon initiating the study, subjects were instructed on how to prepare Hominex®-2 beverages, were given hands-on demonstrations of product preparation, and were allowed to sample a variety of beverage flavors. Subjects were required to give a return demonstration on use of food scales and product mixing to verify their understanding.

Participants continued to meet with the dietitian every 1-2 weeks for the duration of the study. At these sessions, food records were reviewed to enhance accuracy. In addition, subjects were weighed and dietary problems were discussed. Food records from up to 16 weeks were analyzed with First Databank Nutritionist Five™ software.

### Example 5

### Macronutrient Intake

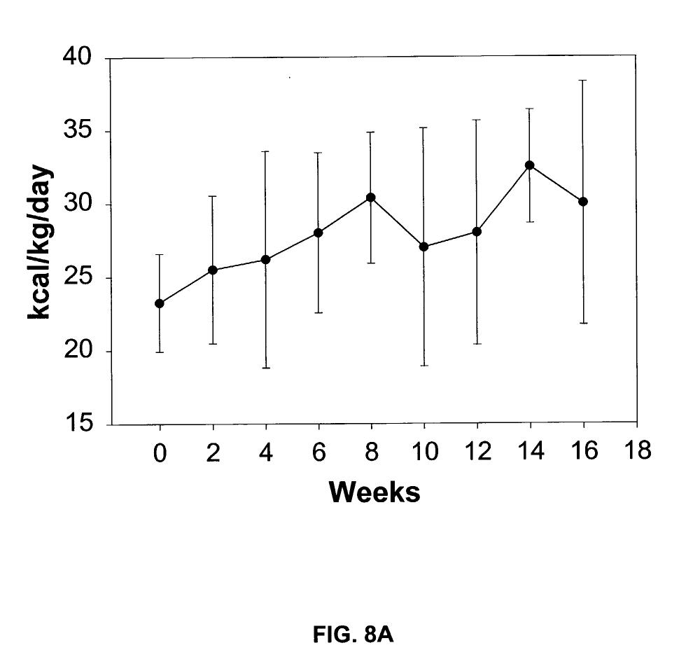

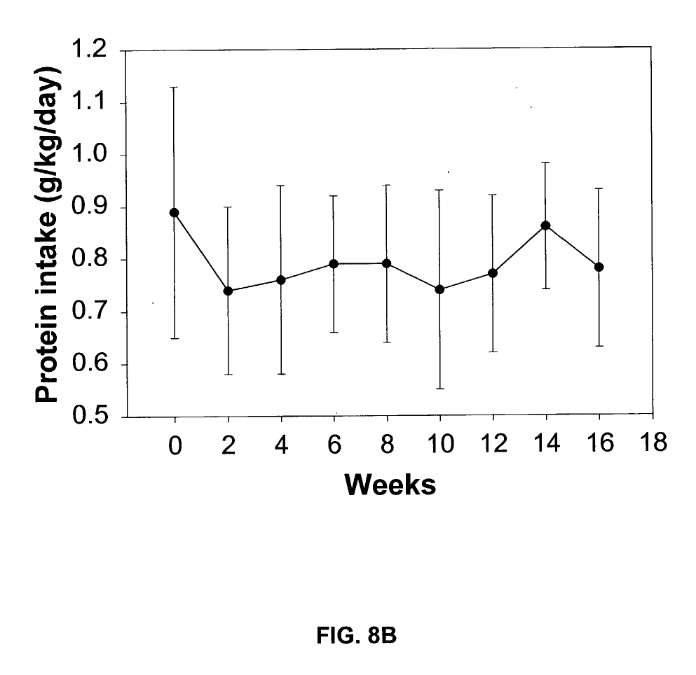

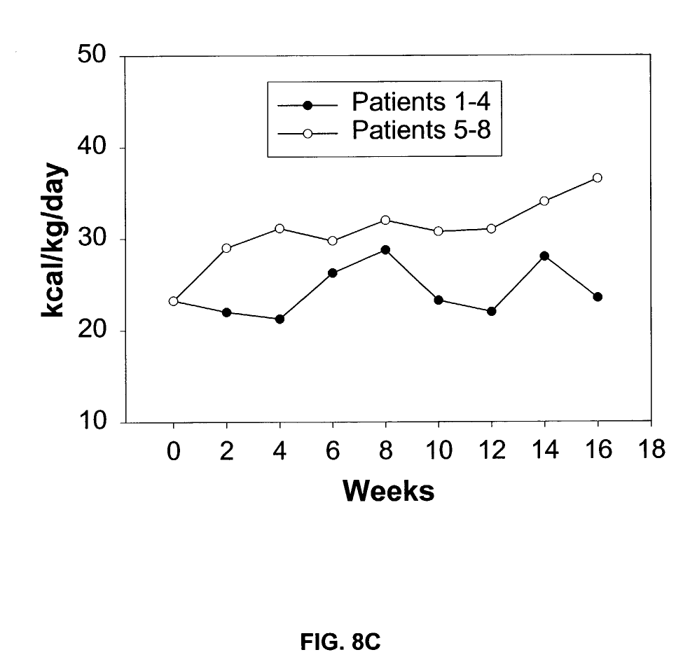

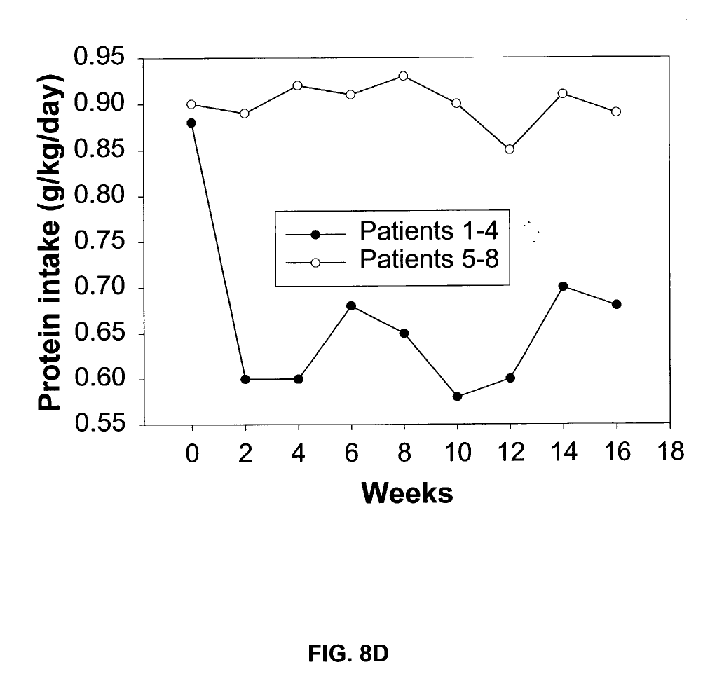

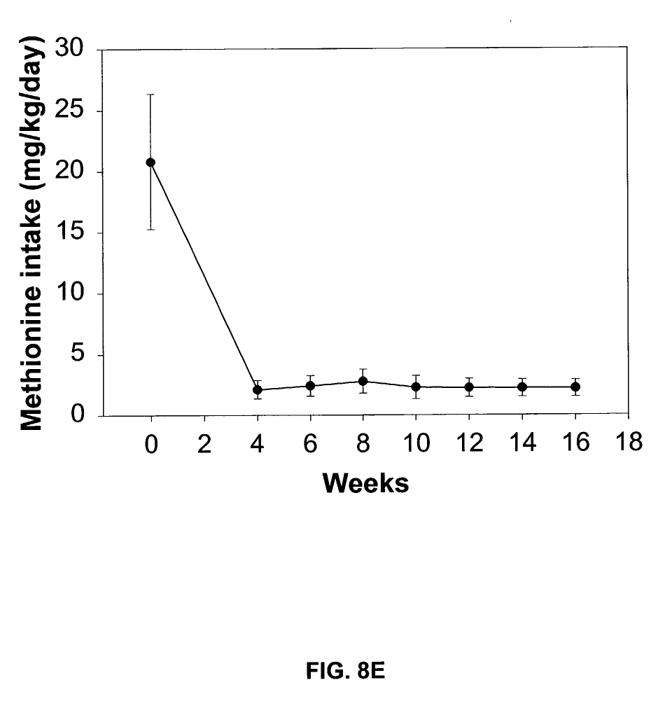

Mean total energy intake during the week prior to beginning the experimental diet was 23.3±3.3 kcal/kg/day and increased during the period of methionine restriction (FIG. 8A). Mean total protein intake was 0.89±0.24 g/kg/day at baseline and was maintained just below the RDA of 0.8 g/kg/day throughout the study (FIG. 8B). However, energy and protein intakes were considerably higher for patients 5-8, who entered after refinements were made in the dietary protocol, than they were for the first four patients (FIGS. 6C and 6D). Methionine intake fell from a mean of 20.8±5.6 mg/kg/day to 2.1±0.74 mg/kg/day upon implementation of the experimental diet, and remained near 2 mg/kg/day, as intended (FIG. 8E).

### Example 6

### Micronutrient Intake

Consumption of lipids, vitamins, and minerals at baseline and during the eighth week of the study are shown in Table 7. Consumption of polyunsaturated fats, vitamins C and E, folate, B12, zinc, and copper all increased significantly (p<0.05) during participation.

TABLE 7
Dietary consumption of lipids, vitamins, and minerals at baseline
and during the eighth week of the study for all 8 participants.

| | Week 0 | | Week 8 | | |
|---|---|---|---|---|---|
| | Mean | S.D. | Mean | S.D. | p-value |
| Saturated fatty acids (g) | 25.63 | 8.37 | 28.63 | 9.5 | 0.315 |
| Monounsaturated fatty | 27.88 | 8.87 | 38.88 | 21.11 | 0.103 |
| acids (g) | | | | | |
| Polyunsaturated fatty | 15.29 | 6.17 | 24.5 | 13.42 | 0.041 |
| acids (g) | | | | | |
| Cholesterol (mg) | 343.5 | 272.79 | 15.11 | 27.6 | 0.008 |
| Vitamin A (μg RE) | 1256.75 | 930.96 | 2007.13 | 888.21 | 0.058 |
| Vitamin C (mg) | 61.15 | 73.63 | 300.25 | 165.72 | 0.009 |
| Vitamin E (mg a-TE) | 7.35 | 3.72 | 35.63 | 14.42 | 0.001 |
| Folate (μg) | 301.13 | 170.25 | 879.88 | 307.43 | 0.001 |
| B12 (μg) | 5.48 | 2.71 | 16.71 | 6.62 | 0.006 |
| Zinc (mg) | 12 | 3.3 | 25.13 | 9.69 | 0.009 |
| Copper (mg) | 1.21 | 0.7 | 2.54 | 0.67 | 0.006 |
| Selenium (μg) | 0.1 | 0.03 | 0.07 | 0.02 | 0.048 |

Cholesterol and selenium intakes declined significantly, whereas consumption of monounsaturated fats, saturated fats, and vitamin A did not change significantly. Average selenium intake during the eighth week still exceeded the RDA (50 μg/day) despite the fact that it declined on the experimental diet as compared to baseline.

### Example 7

### Plasma Amino Acid Levels

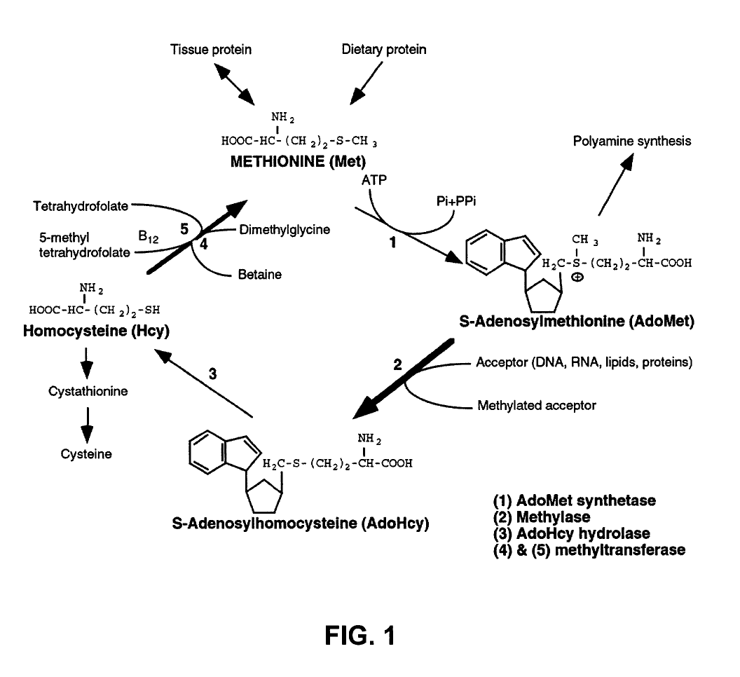

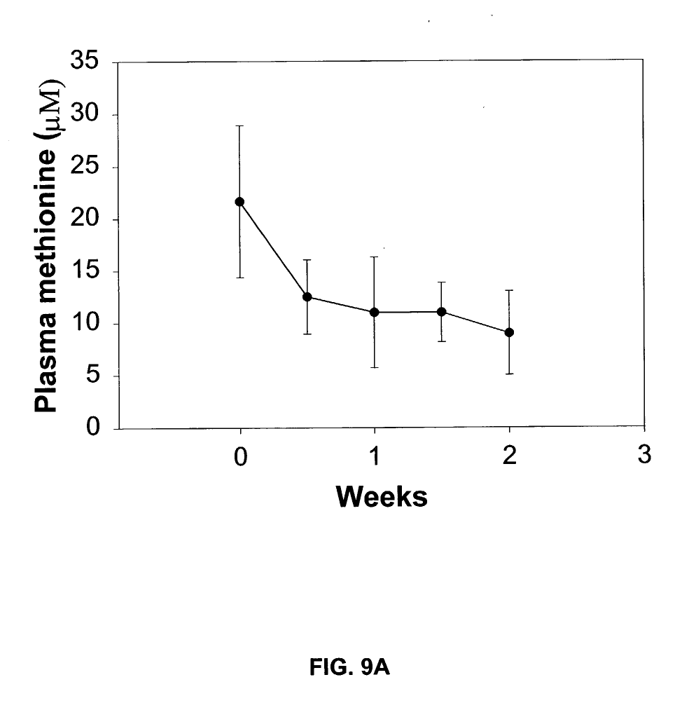

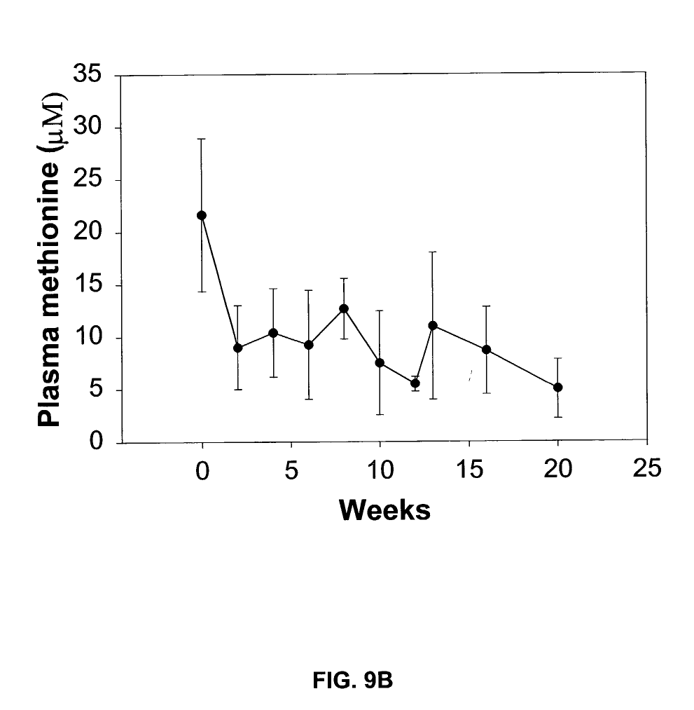

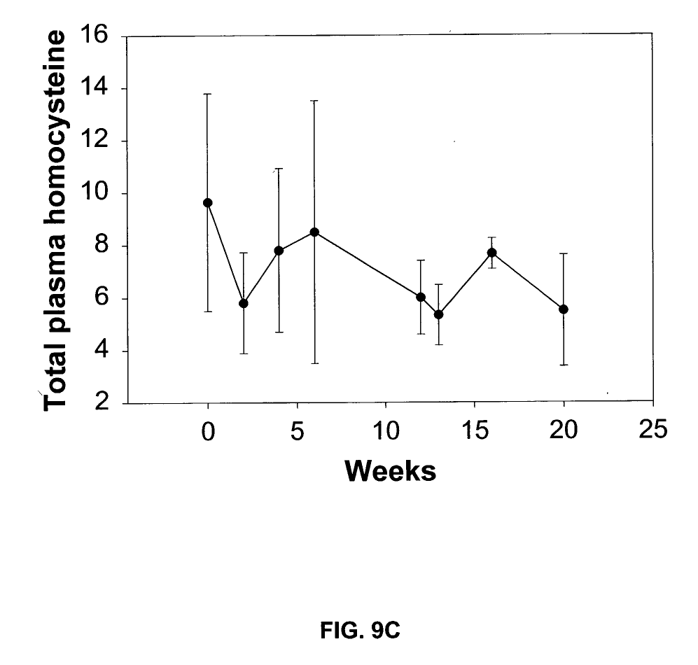

Mean plasma methionine levels decreased from 21.6 to 9 μM within two weeks, representing a 58% decline (FIG. 9A). Plasma methionine levels fluctuated over time but clearly trended downward throughout the study (FIG. 9B). Total plasma homocysteine levels were also measured, since methionine demethylation yields S-adenosylhomocysteine, which can ultimately be remethylated to methionine or converted to cysteine via transulfuration (FIG. 1). Total plasma homocysteine levels trended downward slightly but fluctuated to a greater extent than methionine levels did (FIG. 9C). Levels of other amino acids were not affected significantly by methionine restriction.

### Example 8

### Nutritional Indices

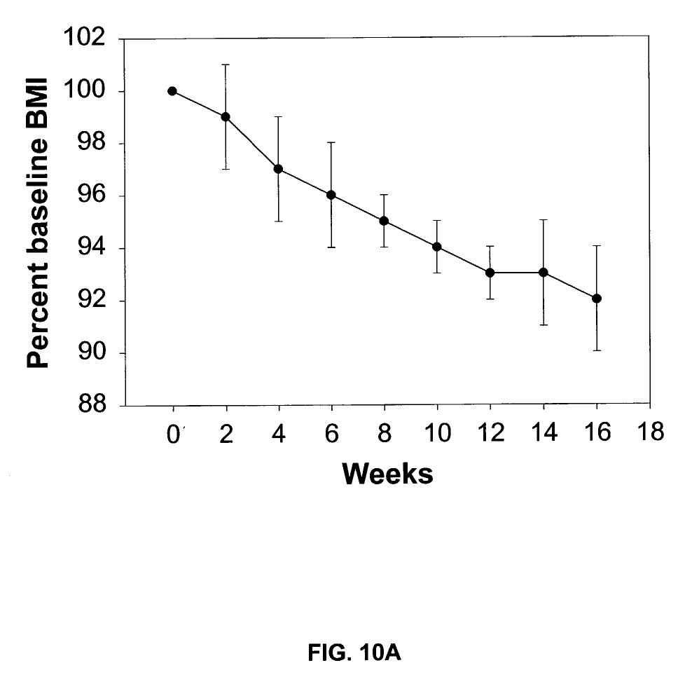

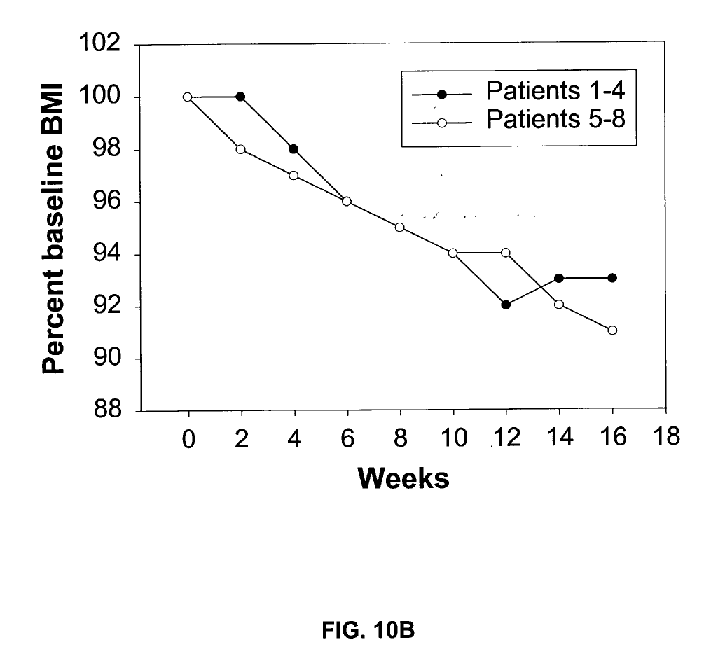

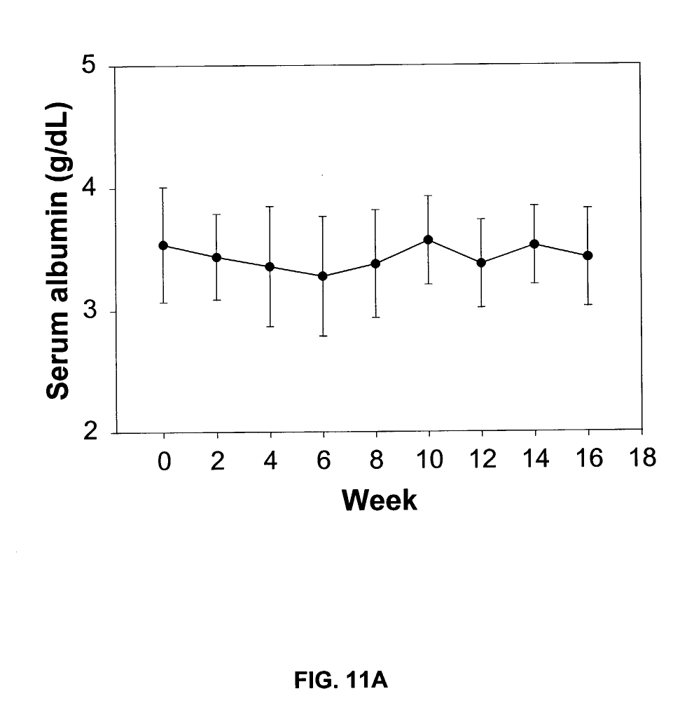

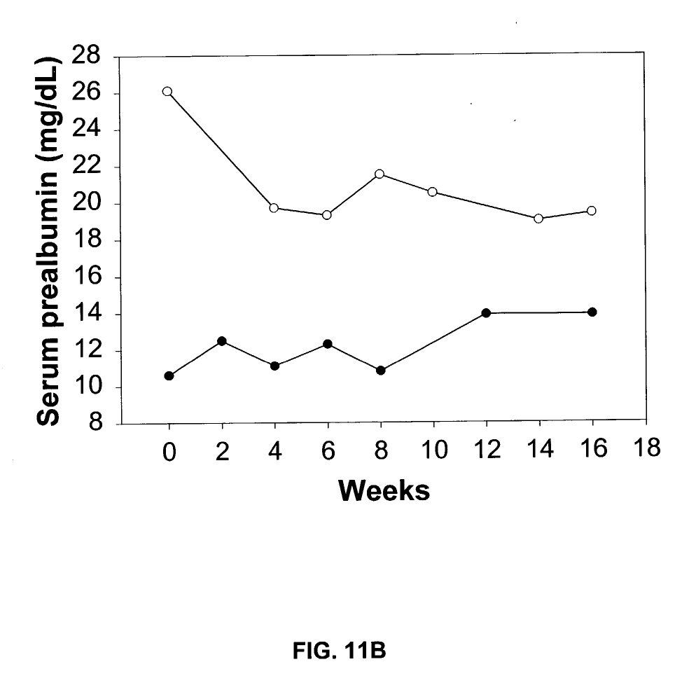

All participants lost approximately 0.5% of baseline BMI (0.5 kg) per week at a constant rate throughout the study (FIG. 10A), regardless of whether they were on the original dietary protocol (patients 1-4, see Example 3) or the refined dietary protocol (patients 5-8) (FIG. 10B). The diet did not significantly affect serum albumin levels (FIG. 11A). Serum prealbumin levels were measured for only two of the patients. Prealbumin declined initially in one of the two patients, but thereafter remained stable and in the normal range, whereas it increased in the other patient over time (FIG. 11B).

### Example 9

### Significance of the Present Invention

The Examples show herein that methionine restriction via an enteral diet for several weeks is safe and feasible in adults with advanced solid tumors and useful for cancer treatment involving methionine restriction alone or in combination with other modalities, such as methioninase or chemotherapy. Weight loss was the only side effect of the diet, and all but one patient regained weight upon resumption of a normal diet. The only patient who failed to regain weight after discontinuing the study had cancer cachexia related to pancreatic adenocarcinoma even before his enrollment. Judging by plasma methionine levels and food records, patients adhered to the diet.

After observing weight loss in the first four patients, the diet was refined in order to provide increased kcal and protein intake. Nonetheless, patients 5-8, who maintained energy intakes considerably above baseline and protein intakes above the RDA, still lost weight at the same rate as the first four patients. One possible explanation for this observation is that 35 kcal/kg/day, which was consumed by patient 5-8, was still inadequate to maintain positive nitrogen balance. This possibility is supported by early studies showing that energy requirements are considerably higher for patients whose sole nitrogen source consists of purified amino acids as compared to those consuming intact proteins (Rose, 1949). Alternatively, weight loss experienced by patients in the trial may have been independent of energy intake but rather attributable to “obligatory” muscle catabolism related to methionine restriction per se. A recent study designed to quantify dietary methionine requirements in normal subjects sheds light on this issue (Raguso et al., 1999). In that study, stable isotope methods were used to measure obligatory methionine oxidation in normal subjects on a diet completely devoid of sulfur amino acids (methionine and cyst(e)ine) for five days. Although somewhat controversial (Young and el-Khoury, 1995), obligatory oxidation rates are considered by many to represent the minimum requirement for amino acids; that is, the amount that is oxidized despite maximal body conservation. The obligatory oxidative loss of methionine was found to be 13 mg/kg/day in that study (Raguso et al., 1999). Patients in the experiments described herein who were restricted to 2 mg methionine/kg/day, therefore, consumed 11 mg/kg/day less than the minimum requirement. However, they consumed adequate amounts of cyst(e)ine, which is present in Hominex®-2. They therefore may have had obligatory methionine oxidation rates less than 13 mg/kg/day. The fact that all patients reversibly lost weight despite what would normally be considered adequate energy and protein intake may actually be encouraging, since it confirms that patients adhered to the diet. The basic premise of this strategy is that dietary methionine restriction will have a greater deleterious effect on tumors than it does on normal host tissues.

Despite the fact that all patients in the current trial lost weight, their serum albumin and prealbumin levels remained stable or increased. Dietary methionine restriction therefore apparently did not cause indiscriminate “starvation”. Although the mechanisms for the antitumor activity of methionine restriction in animals are unclear, in a specific embodiment, they may relate to the specialized functions of methionine. This possibility is supported by studies showing that dietary restriction of any of the other essential amino acids either has no antitumor effect or results in life-threatening toxicity in tumor bearing animals (Sugimura et al., 1959). Methionine functions as a methyl donor for methylation of DNA and other molecules and as an aminopropyl donor for polyamine synthesis. DNA methylation is known to transcriptionally silence several growth inhibitory genes in tumors (Santini et al., 2001), and polyamines have far-ranging effects on nuclear structure and cell division (Polyamines in Cancer: Basic Mechanisms and Clinical Approaches, 1996). Polyamines are more abundant in tumors than they are in corresponding normal tissues.

In Example 7, it was demonstrated that mean plasma methionine levels dropped to 9 micromolar after two weeks on the clinical regimen. This is especially important because it has previously been established (both in vitro and in vivo) and widely reported in the scientific literature that a “therapeutic” range for plasma methionine levels is approximately at or below 10 micromolar. Below 10 micromolar, tumor cells cease to thrive and significant tumor growth inhibition is exhibited. Of further significance is the fact that this drop in plasma methionine was effected by dietary depletion of methionine alone. This would not be expected based on previously published animal studies. In mice and rats, simple dietary depletion of methionine alone was insufficient to drive methionine levels below 10 micromolar. In animal studies, it is necessary to add additional measures to reduce plasma methionine levels into an accepted therapeutic range, including depleting choline, cyst(e)ine, cobalamine, and folate from the diet. Additional measures also include pharmaceutical administration of the enzyme methioninase.

In specific embodiments, the methionine restriction is effective either alone or in combination with chemotherapy, gene therapy, or other modalities.

### Example 10

### Dietary Methionine restriction and Temodal® (SCH52365) for the Treatment of Recurrent and Progressive Malignant Gliomas

A. Current Treatment of Glioblastoma Multiforme (GBM)

Malignant gliomas are rapidly growing primary brain tumors associated with a high degree of morbidity and mortality. Current management is based on cytoreduction through a combination of surgery, radiotherapy and chemotherapy. Despite aggressive multi disciplinary treatment, malignant glioma remains a life-threatening disease.

Overall median survival for patients with newly diagnosed malignant gliomas and Kamovsky performance status (KPS)>60 treated with radiation and chemotherapy after surgery reported in several randomized trials ranges from 42 to 50 weeks for GBM and 182 to 228 weeks for non-GBM malignant gliomas (DeVita et al., 1997). A more recent publication by Fine et al, reported the results of meta-analysis of 16 randomized trials evaluating the use of various chemotherapeutic regimens in patients with newly diagnosed malignant gliomas (Fine et al., 1993). These data suggest that patients with newly diagnosed malignant gliomas may benefit from adjuvant chemotherapy in terms of survival. The most commonly used agent or regimen in these studies included a nitrosourea.

The nitrosoureas (BCNU and CCNU) are chemotherapeutic agents approved for the treatment of patients with malignant glioma at first relapse. Response rate (defined as responders plus stable disease) for single agent BCNU in the treatment of patients with GBM at recurrence has been reported at 29% with a median time to progression of 22 weeks (Berger et al., 1996). The median time to progression for recurrent malignant gliomas following treatment with various other chemotherapeutic agents ranges from 16-50 weeks for anaplastic astrocytoma (AA) (Berger et al., 1996). Response rates (defined as responders plus stable disease) in these same studies have ranged from 0-55% in GBM, 0-95% in AA and 24-50% in GBM and AA (Berger et al., 1996). In addition, the Southwest Oncology Group recently reported their analysis of 9 separate Phase II trials of chemotherapy in recurrent malignant glioma which demonstrated no correlation between response rate and survival (Taylor et al., 1993). From these data, there does not appear to be a correlation between response rate and median time to progression, or between response rate and survival. Not one chemotherapeutic agent has emerged as a standard in the treatment of patients with malignant glioma at relapse.

Temozolomide has demonstrated activity in CRC Phase I and II trials of patients with malignant glioma and a tolerable safety profile (Patel et al., 1995; Newlands et al., 1992; O'Reilly et al., 1992; O'Reilly et all., 1993). Given the paucity of currently available treatments and the lack of an accepted standard treatment at relapse, Temodal® in specific embodiments offers patients an improvement in progression-free survival and/or health-related quality of life. Schering-Plough Research Institute has sponsored a worldwide Phase II trial in patients with anaplastic astrocytomas at first relapse (C/194-123). The study closed to enrollment in October, 1996. Preliminary interim analysis of data suggest that Temodal® has modest activity in this disease. A second study in patients with glioblastoma multiforme in which patients are randomized between Temodal® and procarbazine is ongoing. Early analysis has also suggested clinical activity by Temodal®.

B. Dietary Methionine Restriction Plus Chemotherapy: an Innovative Approach to GBM Treatment

Despite incremental advances in the systemic treatment of GBM in recent years, the outlook for patients with GBM remains poor. Bold innovation will be required to make a major impact on this devastating disease. Recent preclinical studies strongly suggest that depletion of methionine, an essential amino acid, synergistically enhances the efficacy of Temodal® and other chemotherapy drugs in the treatment of glioma (Kokkinakis et al., 2001). The current study is designed to assess the efficacy and toxicity of methionine restriction combined with Temodalg.

C. Optimizing the Sequence and Schedule for Combining Methionine Restriction with Chemotherapy.

Several factors need to be considered in designing possible treatment schemes. First is the optimal duration of methionine restriction. Recent studies indicate that human prostate cancer cells grown in culture medium lacking methionine stop proliferating within 1-2 days, enter a cell cycle arrest for a few days, but do not commit to a death pathway for approximately a week or more (Lu and Epner et al., 2000). Not surprisingly, regression of experimental tumors in animals in response to methionine restriction takes longer than death of cancer cells in culture. Human sarcoma xenografts in mice maintained on a methionine free diet continue to grow for about two weeks, stabilize for another two weeks, and ultimately regress thereafter (Guo et al., 1993). Extrapolating from laboratory studies to humans, as is standard in the art, in a specific embodiment several weeks of methionine restriction are required to achieve tumor regression in humans, despite the fact that plasma methionine levels often fall significantly in humans within just a few days. Results from the Examples presented herein suggest that about 12 weeks of restriction will be required to produce antitumor activity. A skilled artisan is aware of how to alter and coordinate the diet regimens in order to achieve a therapeutic effect.

A second factor to consider is the maximal length of time that patients will be able to tolerate dietary methionine restriction. Whereas one patient remained on the experimental diet for 39 weeks, most patients expressed the strong desire to come off study after 16 weeks even if they experienced stable disease or responded. Twelve to 16 weeks therefore seems like a reasonable duration for the current study, which is the first clinical trial involving dietary methionine restriction combined with chemotherapy.

Another question is whether to intentionally cycle patients off the restricted diet briefly at regular intervals (i.e. after 12 weeks of restriction), in much the same way as cancer patients are treated with “cycles” of chemotherapy. Allowing patients who are on methionine-restricted diets to resume normal diets briefly at regular intervals would accomplish this. Besides being more tolerable for patients, such an approach, in a specific embodiment, is more effective than chronic restriction. Previous studies from our lab and others indicate that a variety of human tumors arrest in the G2 phase of the cell cycle in response to methionine restriction (Lu and Epner, 2000; Guo et al., 1993). Cancer cells that are forced to leave G2 and reenter the cell cycle prematurely following exposure to chemotherapy die much more rapidly than those remaining in G2 (Chan et al., 1999; Suganuma et al., 1999). Such “abrogation” of G2 cell cycle arrest accelerates cancer cell death. Intentionally repleting methionine after a period of depletion therefore enhances the antitumor activity of methionine restriction by forcing cancer cells to prematurely reenter the cell cycle. This is supported by results illustrated in Examples 1-8. One patient with recurrent renal cell carcinoma who was on the experimental diet for 39 weeks experienced an unequivocal radiographic response only after resuming a normal diet. Another patient with metastatic carcinoid who was on the diet for 16 weeks also experienced a subtle radiographic response several weeks after resuming a normal diet. These results support the “cycling” concept.

Another consideration is the optimal sequence of dietary methionine restriction and chemotherapy. One approach would be to treat initially with temozolamide alone to sensitize tumors to subsequent methionine restriction. The advantage of this approach is that chemotherapy will be given promptly. However, this sequence is inconsistent with preclinical studies in which tumor-bearing animals were methionine-restricted before receiving chemotherapy (Kokkinakis et al., 2001). Another approach which is more consistent with animal studies is completion of a 12 week cycle of methionine restriction prior to giving the first chemotherapy. However, this approach would have the disadvantage of deferring chemotherapy, which has some antitumor activity alone. A third approach would be to begin dietary methionine restriction and temozolamide simultaneously. The practical disadvantage of this approach is that patients will need to begin oral temozolamide, which is emetogenic, and a somewhat complex dietary regimen concomitantly.

D. Temodal®

Although a skilled artisan recognizes any chemotherapeutic drug or alternative cancer therapy may be administered in addition to the diets described herein, in a specific embodiment, Temodal® is given in addition to the diet of the present invention. Temodal® is 4-Dihydro-3-methyl-4-oxoimidazo-[5,1-d]-1,2,3,5-tetrazin-8-carboxamide. (Former name includes 8 carbamoyl-3-methylimidazo-[5,1-d] 1.2.3.5-tetrazin-4-(3H)-one.) Temodal® (Temozolomide, SCH 52365) is an oral alkylating agent of imidazotetrazine derivatives, which exhibits broad-spectrum antitumor activity against murine tumors (Stevens et al., 1984). Temodal® was developed as a potential alternative to dacarbazine in view of its demonstrated antitumor activity and better toxicity profile in pre-clinical testing (Tsang et al., 1991; Bull and Tisdale, 1987; Tsang et al., 1990; Clark et al., 1990). Both compounds are cytotoxic alkylating agents whose active metabolite is the linear triazine, monomethyl triazenoimidazole carboxamide (MTIC) (DeVita et al., 1991; Stevens et al., 1987). The cytotoxicity of MTIC is thought to be primarily due to alkylation at the 06 position of guanine (O'Reilly et al., 1993) with additional alkylation also occurring at the N7 position (Hartley et al., 1986). Whereas dacarbazine requires metabolic activation by the liver, temozolomide undergoes chemical degradation to MTIC at physiologic pH (Tsang et al., 1990; Stevens et al., 1987).

E. Dietary Methionine Restriction-“Dose Escalation” Schema

Based on experiments described in Examples 1-8, it is known that patients with metastatic cancer tolerate dietary methionine restriction without chemotherapy for at least 12-16 weeks at a time. In some embodiments, a dietary methionine restriction for 12-16 weeks is administered in combination with temozolamide. Therefore, a dietary “dose escalation” is performed as described and illustrated below. The temozolamide regimen (daily X 5 on days 22-26 of each 28 day period) is the same regardless of which dietary schedule a patient follows.

All patients consume a methionine free diet during the first 2 weeks. The first five patients are then switched to a methionine restricted diet containing <2 mg/kg/day methionine for the third week, followed by a “replete” diet containing 20-25 mg/kg/day methionine during the fourth week. This four week cycle is repeated for the first five patients throughout their participation.

If patients 1-5 tolerate the four week cycle, then the period of methionine restriction is lengthened such that patients 6-10 are on the methionine free regimen for the first two weeks, followed by the 2 mg/kg/day regimen for five weeks (weeks 3-7), followed by a replete diet during week 8. This 8 week schedule will be repeated throughout participation.

If patients 6-10 tolerate the eight week cycle, then the period of methionine restriction is further lengthened such that patients 11-15 are on the methionine free regimen for the first two weeks, followed by the 2 mg/kg/day regimen for nine weeks (weeks 3-11), followed by a replete diet during week 12. This 12 week schedule will be repeated throughout participation.

If patients 11-15 tolerate the 12 week cycle, then Patients 16-20 are on the methionine free regimen for the first two weeks, followed by the 2 mg/kg/day regimen for 13 weeks (weeks 3-15), followed by a replete diet during week 16. If this 16 week cycle is tolerated by all five patients, then all subsequent patients will follow the 16 week cycle throughout participation.

Patients are considered to be “intolerant” of a particular dietary regimen (cycle length) if they are unable to meet energy or protein intake goals for more than two weeks at a time, experience persistent nausea or vomiting, or lose more than 1% of their body weight per week. During two week periods of methionine free diet, patients will consume only “shakes” containing Hominex-2® that are easy to prepare, palatable, and nutritionally complete (i.e. provide 35 kcal/kg body weight/day). Patients will eat 2.67 g Hominex-2®/kg/day, which will provide 0.8 g protein and 11 kcal/kg/day. Plasma amino acid profiles will be obtained weekly during the first three weeks and every other week thereafter. The dietician provides written instructional materials and individualized food plans to help patients maintain methionine intakes <2 mg/kg/day during the variable periods of methionine restriction. Patients will be able to reintroduce some “regular” foods into their diets if they so desire during this phase.

A skilled artisan recognizes that dose modifications of Temodal® may need to be adjusted, and that recovery is achieved following achievement of specific conditions. Furthermore, a pretreatment evaluation is desirable for all patients, including CBC, differential, platelets, total protein, albumin, calcium, phosphorus, glucose, BUN, creatinine, uric acid, total bilirubin, alkaline phosphatase, LDH, SGPT, electrolytes, anticonvulsant levels, and Gd-DPTA MRI scan. The criteria for response and toxicity will be pre-determined, such as a clinical neurological examination and MRI brain scan.

### Example 11

### Intact Protein Source for Methionine-Restricted Diet

In a specific embodiment of the present invention, the methionine-restricted diet comprises an intact protein source having low methionine levels. A skilled artisan recognizes that there are multiple avenues with respect to providing a source of intact protein(s) suitable for a low-methionine diet. In a specific embodiment, the proteins are screened for in whole foods, whereas in another embodiment the proteins are targeted specifically. In one embodiment, the proteins are recombinantly produced. In a specific embodiment, the methionine level is about 0.2% methionine by weight of total protein.

With regard to the first approach, a search of United States Department of Agriculture (USDA) nutritional database indicated chayote, strawberries, tea powder, pea pods, instant coffee powder, custard apples, grape juice, and orange juice have methionine levels of <0.2% (w/w). However, the first five were tested by standard means in the art and found to all be>0.3%. Methionine levels were determined by acid hydrolysis of proteins followed by chromatographic separation of the individual amino acids. Quantitation was achieved using ninhydrin to form UV-absorbing complex with the amino acids. In addition to the standard amino acids, the oxidation products of methionine (methionine sulfoxide and methionine sulfone) were also identified and quantified. Methionine levels reported are the sum of methionine, methionine sulfoxide and methionine sulfone. Total protein content was determined with a semi-automated nitrogen analyzer which utilizes an adaptation of the classical acid digestion/ammonia distillation procedure of Kjeldahl (1883). The juices and limes have very low total protein and would be prohibitively expensive as dietary protein sources. Custard apples are similarly tested. It is a preferred embodiment of the present invention to utilize a whole food, preferably which is easily obtainable and have a low degree of processing to remain affordable.

In another approach, purified protein(s) are utilized in the diet of the present invention. In a specific embodiment, proteins are purified from a legume, such as garden peas and/or broad beans.

For garden peas, two sets of experiments have produced significant results. In the first set of experiments, purification of vicilin was carried using a modification of the scheme depicted in FIG. 2. This is a standard approach that skilled artisans have used to obtain the various protein fractions from pea seeds. Garden pea seeds (either powdered or intact) were mixed with buffer at pH 7 containing 0.3 M NaCl homogenized. Following homogenization, the mixture was stirred overnight at 4° C. Insoluble debris was removed by centrifugation (4000×g for 30 minutes). The resulting supernatant was placed in a dialysis bag (1000 dalton MWCO) and dialyzed against water overnight at 4° C. The material retained in the dialysis bag was centrifuged, and the resulting supernatant placed in a new dialysis bag and dialyzed overnight at 4° C. Again, insoluble material was removed by centrifugation. Insoluble material from the two water dialyses was combined and solubilized with buffer at pH 7 containing 0.2 M NaCl. The resolubilized material was dialyzed overnight at 4° C. against 0.1 M sodium acetate, 0.2 M NaCl, pH 4.7. Insoluble material was removed by centrifugation. The resulting supernatant contains the vicilin fraction. The methionine content of this fraction has been as low as 0.2%.

In the second experiment, the extract was prepared as above through centrifugation to remove debris. The supernatant was then passed through a 25 micron membrane, the resulting filtrate passed through a 0.2 micron membrane, the 0.2 micron filtrate passed through a 0.1 micron membrane. The material that passed through the 0.2 micron membrane but was retained by the 0.1 micron membrane had a methionine content of 0.18%. The 0.1 micron retentate contained 23% of the protein present in the starting extract. This filtration approach is scalable and commercializable.

In the garden pea, the vicilin fraction has a methionine content of 0.05% while in the broad bean it is 0.1%. However, the pea legumins have a methionine content of 0.5% while those of the broad bean have only 0.2%. A skilled artisan recognizes that a difficult facet of seed storage protein purification is the separation of legumins and vicilins. According to the literature, vicilins and legumins are present in equal abundance within the broad bean and thus methionine content of these combined fractions should be about 0.15%, which is within the desirable quantity for the present invention. Initial work in this area has suggested filtration similar to that described above for the garden pea should work with broad beans. However, the products of these preliminary investigations had higher than expected methionine content. Identification of specific broad bean legumins by mass spectroscopy and N-terminal sequencing has indicated the strain used in the current experiments appears to have one particular legumin of high methionine content. Thus, additional strains are screened by the methods described above to identify a more suitable candidate.

### Example 12

### Manufacture of Elemental Nutritional Composition

In some embodiments of the present invention, an elemental nutritional composition is generated. For example, initially a “base powder” is created which contains all the fat, carbohydrate, fiber, vitamins and minerals. Generally, a composite slurry of all of these components are spray-dried in a large drying tower. The resultant base powder is then put into a large blender where the amino acids (as pure crystalline powders) are individually dry-blended into the base powder, and this results in the final product (i.e. Hominex-2). In the Tumorex embodiment, some artificial sweeteners and flavoring agents were also dry-blended in as the final step of the processing.

### Example 13

### Involvement of Caspases in Mediating programmed Cell Death

Studies for the present example were undertaken to elucidate the molecular pathways leading to apoptosis induced by methionine restriction. In particular, activation of a mitochondria-dependent cell death pathway by methionine restriction was studied.

Growth of Hela human cervical carcinoma cells in culture in medium containing homocysteine in place of methionine resulted in apoptosis, as indicated by a typical nucleosomal ladder on agarose gel. Similar results were seen following methionine restriction of PC-3 cells (Lu and Epner, 2000). Cytochrome c release from mitochondria was next measured to determine whether apoptosis in response to methionine restriction is mitochondria-dependent. Methionine restriction increased cytosolic levels of cytochrome c in both Hela and PC-3 cells, implicating mitochondria-dependent pathways. Furthermore, transfection of either Bcl-2 or Bcl-XL, two molecules known to inhibit apoptosis by blocking cytochrome c release from mitochondria and activation of caspases (Reed, 1998), inhibited cell death in response to methionine restriction plus JNK1 overexpression.

It was next examined whether methionine restriction caused activation of caspases, including initiator caspases 8 and 9 and effector caspases 3 and 6. Methionine restriction had little if any effect on levels of these caspases in PC-3 cells. In contrast, caspase 3, 6, and 9 proenzyme levels decreased upon methionine restriction in a time-dependent fashion in Hela cells. Caspase 8 proenzyme levels were unaffected in both cell lines. Despite the fact that caspase 3, 6, and 9 proenzyme levels fell in Hela cells in response to methionine restriction, the active forms of all three caspases were undetectable by western blot analysis. Inability to detect cleaved, active caspases was not due to technical problems with the western blot procedure, since the active form of caspase 9 was detectable following treatment of Hela cells with staurosporine, a compound known to rapidly induce apoptosis. In a specific embodiment, active forms of caspases were not detectable as a result of their lability and rapid degradation relative to the very gradual pace of cell death induced by methionine restriction.

Consistent with the western blot analyses, caspase activities increased in Hela cells in response to methionine restriction. Cleavage of peptide Ac-LEHD-AFC, which is a substrate for caspases 4, 5, and 9, increased transiently after six days of methionine restriction. Although caspase 4 and 5 levels were not measured by western blot, the observed increase in Ac-LEHD-AFC cleavage after six days of methionine restriction was probably largely a reflection of caspase 9 activity, since caspase 9 proenzyme was degraded at the same time point. Cleavage of peptide Ac-DEVD-AFC, which is a substrate for caspases 3, 6, 7, 8, and 10, increased in a time-dependent manner in Hela cells in response to methionine restriction for up to 15 days. This increase is probably largely a reflection of caspase 6 activity, since caspase 6 proenzyme was almost entirely degraded in Hela cells with similar kinetics. However, caspases 3, 7, 8, and 10 may have also contributed. In contrast to the Hela cell data, caspase activity was not detected in PC-3 cells in response to methionine restriction. These data suggest that Hela cells undergo caspase-dependent cell death, whereas PC-3 cells undergo caspase-independent cell death in response to methionine restriction.

To determine the underlying mechanisms of crosstalk between the stress response and mitochondria-dependent cell death pathway, levels of BID and Bcl-2 were measured in response to methionine restriction. The cleaved, active form of BID is known to induce apoptosis by binding to mitochondria, thereby releasing cytochrome c. BID is also cleaved to its active form as a consequence of JNK1 activation (Tournier et al., 2000). Methionine restriction induced cleavage of BID in a time-dependent manner in Hela cells and, to a lesser extent, in PC-3 cells. Staurosporine-treated Hela cells were used as a positive control for BID cleavage (Tang et al., 2000). In addition, methionine restriction resulted in a slight decrease in Bcl-2 protein levels in PC-3 and Hela cells. However, Bcl-2 did not become phosphorylated (inactivated) in response to methionine restriction. In contrast, Bcl-2 was phosphorylated, as indicated by an upward shift on western blot, in taxol-treated PC-3 and Hela cells, which served as positive controls for Bcl-2 phosphorylation (Haldar et al., 1997; Haldar et al., 1995) (FIG. 5B, lower panel). These data suggest that activation of BID and decreased Bcl-2 abundance led to initiation of the mitochondria-dependent cell death pathway.

Experimental procedures for this example were as follows:

Reagents—Antibodies for caspases 3, 6, 8, and 9, and BID, were purchased from Santa Cruz Biotechnology, Inc. (Santa Cruz, CA). Antibody for cytochrome c was obtained from BD PharMingen (San Diego, Calif.).

Hypotonic lysis for cytochrome c western blot analysis—Determination of cytochrome c release from mitochondria upon activation of apoptosis was performed as described (15). Cells were trypsinized, pelleted at 1500 rpm for 5 minutes, washed twice with PBS, lysed in 300 μl hypotonic buffer (220 mM mannitol, 68 mM sucrose, 50 mM PIPES-KOH (pH 7.4), 50 mM KCl, 5 mM EDTA, 2 mM MgCl₂, 1 mM DTT, protease inhibitors), and incubated on ice for 45 minutes. The lysates were homogenized by trituration 20 times and centrifuged at 14000 rpm for 10 minutes. Supernatants were collected and subjected to protein quantitation, followed by western blot analysis using an anti-cytochrome c antibody.

Caspase Assay—Caspase assays were performed according to the manufacturer's protocol (BioRad Laboratories). Briefly, the assays were based on the ability of activated caspase to cleave a fluorogenic peptide containing caspase cleavage site, consequently releasing the fluorogenic molecule. Fluorogenic tetrapeptide Ac-LEHD-AFC is substrate for caspases 4, 5 and 9, whereas fluorogenic tetrapeptide Ac-DEVD-AFC is substrate for caspases 3, 6, 7, 8, and 10 (Calbiochem-Novabiochem Corporation, San Diego, Calif.).

### Example 14

### Methionine Restriction, Folate Metabolism, and Thymidylate Synthase

Methionine is the immediate precursor of S-adenosylmethionine (SAM), the major methyl donor for methylation of DNA, RNA, and other molecules. Methionine metabolism is also intimately related to folate metabolism, since folate-derived methyl groups are required for biosynthesis of methionine. Therefore, folate serves as a bridging molecule for nucleotide and methionine biosyntheses. To further characterize these mechanisms, the inventors undertook the current studies to test the hypothesis that deprivation of methionine (in the absence of folinic acid) reduces intracellular folate levels in methionine dependent prostate cancer cells, resulting in an imbalanced nucleotide pool. The effect of methionine restriction on levels or enzymatic activity of thymidylate synthase was also investigated.

Methionine Restriction Reduced Intracellular Folate Levels.

Intracellular 5,10-methylene-THF levels were first measured in prostate cancer cells in order to investigate whether methionine restriction diverted folate to methionine synthesis, as it does in normal liver (5) and kidney cells (4) as a result of the “methyl folate trap” (6). Methionine deprivation reduced the level of 5,10-methylene-THF by 75% within 24 hours. The effect was maintained for up to 72 hour. As a control, intracellular 5,10-methylene tetrahydrofolate levels were also measured in response to folate depletion for 24 h. As expected, 5,10-methylene tetrahydrofolate fell by 67% in PC3 cells cultured in folate-free medium. These results suggest that one mechanism by which methionine restriction induces prostate cancer cell cycle arrest and eventual apoptosis is by depleting 5,10-methylene tetrahydrofolate, which is a critical precursor for nucleotide biosynthesis. The data are consistent with those of Machover et al, who also showed that methionine restriction reduced 5,10-methylene tetrahydrofolate levels in cancer cells (18). Both studies suggest that synergy between 5FU and methionine restriction is due to folate depletion rather than accumulation, regardless of whether folinic acid is present.

Methionine Restriction Inhibited TS Activity.

It was next determined whether methionine restriction inhibited thymidylate synthase in prostate cancer cells, as it is known to in leukemia cells (18). It was found that methionine restriction inhibited TS activity in PC-3 cells by approximately 40% within 24 hours and by 80% in 48 hours. In contrast, TS activity in normal human prostate epithelial cells was unaffected by methionine restriction, which is a reflection of the greater methionine dependence of cancer cells relative to corresponding normal cells.

The observed fall in TS activity in prostate cancer cells in response to methionine restriction was accompanied by a commensurate fall in TS protein levels by 80% within 24 hours. TS was almost undetectable within 48 hours. These results suggest that inhibition of TS activity in response to methionine restriction was largely, if not entirely, due to reduced TS abundance rather than enzyme inactivation.

In contrast to the observed fall in TS abundance, global cellular protein synthesis, as measured by tritiated leucine incorporation, was not significantly affected by methionine restriction within the first 24 h. In fact, leucine incorporation during the first six hours of the experiment was actually greater in cells deprived of methionine than it was in control cells. The observed fall in TS protein abundance was therefore not simply due to global protein synthesis inhibition but rather to specific down-regulation of TS levels through mechanisms that we have not yet defined.

As a control, TS enzymatic activity was measured in PC3 cells in response to 5-FU. As expected, 5-FU inhibited TS activity by 95% within 8 hours, whereas it dramatically increased TS protein level as determined by western blot. The observed TS protein accumulation in response to 5-FU was largely abrogated by concurrent methionine restriction. These results suggest that synergy between methionine restriction and 5-FU may at least partially be due to blockage of TS up-regulation, which is a major mechanism by which cancer cells become resistant to 5-FU (26;27).

Methionine Restriction Disrupted Nucleotide Balance.

HPLC was used to measure the effect of methionine restriction on nucleotide levels in PC-3 cells, since TS plays a central role in nucleotide biosynthesis. The ratio of dUMP to dTMP rose from 0.48±0.07 pmole/million cells at baseline to 1.75±0.61 pmole/million cells after 24 h of methionine restriction and remained at about the same level for up to 48 h. 5-FU treatment was used as a positive control for TS inhibition, and, as expected, resulted in a dramatic increase in dUMP/dTTP ratio. These data suggest that one of the mechanisms by which methionine restriction induces prostate cancer cell cycle arrest and eventual apoptosis is by inhibiting TS and thereby disrupting nucleotide balance. We are currently exploring other potential mechanisms for the selective antitumor activity of methionine restriction.

Experimental procedures for the present example were as follows:

Cell Culture

Human prostate cancer PC3 cells (American Type Culture Collection, Rockville, Md.) were maintained in RPMI-1640 (Life Technologies, Inc., Gaithersburg, Md.) supplemented with 10% FBS (HyClone Laboratories, Logan, Utah) at 37° C. in 5% CO₂. Methionine restriction experiments were performed in methionine-free RPMI-1640 (Life Technologies, Inc.) supplemented with 10% FBS and 100 μM Homocysteine (Sigma, St. Louis). Folate restriction experiments were performed in folate-free RPMI-1640 medium (Life Technologies, Inc.) supplemented with 10% FBS. Primary culture prostate epithelial cells (PrEC) were purchased from BioWhittaker, Inc. and Clonetics Products (Walkersville, Md.). PrEC was maintained in the prostate epithelial basal medium and methionine restriction was performed in prostate epithelial cell labeling medium without methionine supplemented with 100 μM homocysteine, which were obtained from the company.

Reagents

Antibody for TS was obtained from Lab Vision Corporation (Fremont, Calif.). 5,10-methylene-THF was purchased from Schircks Laboratories (Jona, Switzerland). [5⁻³H]-dUMP was from Amersham (Amersham Co., Arlington Height, Ill.).

Western Blot Analysis

Aliquots of samples with 50 μg of protein, determined by the Bradford assay (BioRad, Hercules, Calif.), were mixed with loading buffer (final concentrations of 62.5 mM Tris-HCl (pH 6.8), 2.3% SDS, 100 mM dithiothreitol, and 0.005% bromophenol blue), boiled, fractionated in a 10% SDS-PAGE, and transferred onto a 0.45-μm nitrocellulose membrane by electroblotting (BioRad). The membranes were blocked with 2% fat-free milk in PBS, and then probed with first antibody (0.05 μg/ml IgG) in PBS containing 0.1% Tween 20 (PBST) and 1% fat-free milk. The membranes were then washed four times in PBST and incubated with horseradish peroxidase-conjugated F(ab′)₂ of secondary antibody (BioRad) in PBST containing 1% fat-free milk. After washing four times in PBST, the membranes were visualized using the ECL Western blotting detection system.

Thymidylate Synthase (TS) Assay

TS assay was performed as previously described (24). Briefly, 25 μl of cell extract containing 50 μg protein, 5 μl 6.5 mM 5,10-CH₂-THF, and 10 μl of Tris.HCl buffer were combined at room temperature. The assay was initiated by addition of 10 μl [5-3H]-dUMP 1 uM (1.0 mCi/ml, Amersham Pharmacia Biotech), incubated for 30 min at 37° C., and stopped by addition of 50 μl ice-cold 35% trichloroacetic acid and 250 μl of 10% neutral activated charcoal. After centrifugation, 150 μl of the supernatant were counted by liquid scintillation. TS activity was proportional to the amount of tritium released from [5⁻³H]-dUMP into solvent upon dTMP formation.

5,10-methylene Tetrahydrofolate Assay

Intracellular 5,10-methylene-THF was measured by the standard TS assay as described above except that the amount of commercial TS was kept constant at X and cellular folate extracts containing unknown amounts of 5,10-methylene-THF were added to the standard reaction mixture. Release of tritium into the solvent in this assay therefore reflected 5,10-methylene-THF levels rather than TS activity. Folate extraction was performed as previously described (18). Briefly, cells were suspended in cold buffer [50 mM Tris-HCl (pH 7.4), 50 mM sodium ascorbate, and 1 mM EDTA] to a density of 10 to 20×10⁶ cells/ml. Cells were lysed in a boiling water bath for 3 minutes and centrifuged at 14,000×g for 5 minutes at 4° C. The supernatant was used immediately for the assay or frozen at −70° C. until used.

Measurement of Total Cellular Protein Synthesis

2×10⁵ of PC3 cells were seeded per well in 6-well plates. The next day, 3 ml of either complete medium or methionine-free medium containing 1 μl of H³-leucine (1.0 mCi/ml, Amersham Pharmacis Biotech) were added into each well at 1, 3, 6, 24 hours before harvest of the total cellular protein. The cells were then washed with PBS twice and lysed in 100 μl of lysis buffer (20 mM Tris/HCl, pH 8.0; 137 mM NaCl; 10% w/v glycerol; 10 mM NaF; 1% Triton X-100; 1 mM Na₃VO₄; 2 mM EDTA; 1 mM PMSF; 20 μM leupeptin; and 0.15 units/mL aprotinin). The total cellular protein was then concentrated by TCA precipitation. The samples containing 10% TCA were incubated on ice for 30 min and spun at 14,000 g for 5 min. The precipitated protein was dissolved in 50 ml of 0.1 M NaOH. Radioactivity was determined by Liquid Scintilation Counter.

Preparation of Cellular dNTP Extract for HPLC Analysis (25)

Cells were washed with phosphate buffered saline (PBS), trypsinized, washed again with PBS, counted, and pelleted. 5×10⁵ cells from each sample were mixed with 10 μL 0.6 M trichloroacetic acid. The lysate was incubated at 4° C. for 30 minutes. After centrifugation, the acidic supernatant was transferred to a microcentrifuge tube. An equal volume of ice cold 80% 1,1,2-trichlorotrifluoroethane and 20% tri-n-octylamine was added to the lysate. The mixture was vortexed for 15 seconds and then centrifuged at 14000 g at 4° C. for 5 minutes. The aqueous supernatant was removed and centrifuged at 14000 g at 4° C. for 5 minutes. Samples were stored at −70° C. until use.used.

HPLC analysis

Clromatographic analyses were performed with a Waters 625 LC System (Waters Corporation, Milford, Mass., USA) consisting of a Waters 625 Fluid Handling Unit with a Rheodyne 9125-080 Manual Injector and 20 μL sample loop, 625E Powerline Controller, and 484 Tunable UV Detector. Component separation was achieved using a reversed phase SS Exsil ODS column (5 μM particle size, 4.6×250 mm, SGE Incorporated, Austin, Tex., USA). The column was maintained at ambient temperatures. The methodology of Cross, et al. with some modification was used to separate the nucleotides (25). Briefly, two buffers comprised the mobile phase—Buffer A consisting of 0.2 M (NH₄)H₂PO₄ in 1.0 M KCl at pH 5.35, and Buffer B consisting of 0.2 M (NH₄)H₂PO₄ in 1.25 M KCl and 10% methanol at pH 5.0. pH was adjusted with NaOH solution and Buffer B was titrated after the addition of methanol. UV detection was at 250 nm. Solvent flow rate was maintained at 0.8 ml/minute during the elution gradients. The elution gradients were as follows: 100% Buffer A for 8 minutes followed by a 13 minute linear gradient to 75% Buffer A and 25% Buffer B. At 22 minutes, a 2 minute linear gradient to 15% Buffer A and 85% Buffer B started. 15% Buffer A and 85% Buffer B was maintained until the end of the run at 40 minutes. Afterwards, the column was regenerated with 100% Buffer A at 1.0 ml/minute for 15 minutes.

A series of standards containing varying amounts of dUMP and dTTP ranging from 1.0˜0.02 mmoles was analyzed using the above methodology. The different quantities and their correlating absorption areas existed in a linear relationship. Using the least squares method, a linear equation was generated. This linear equation was used to calculate the quantity of dUMP or dTTP represented by the absorption peaks in the chromatograms generated from our experimental samples.

### Example 15

### Dietary Restriction on Prostate Cancer Progression in a Mouse Model

The effects of dietary methionine restriction on prostate cancer progression is studied in a transgenic mouse prostate model, the “TRAMP” model (Greenberg et al., 1995). Autochthonous transgenic adenocarcinoma mouse prostate (TRAMP) model is well known in the art to facilitate characterization of molecular mechanisms involved in the initiation and progression of prostate cancer. TRAMP mice display high-grade prostatic intraepithelial neoplasia or well-differentiated prostate cancer by 10-12 weeks of age.

In specific embodiments of the present invention, a diet as described elsewhere herein is administered to the transgenic mouse and compared to control mice for assessment of antitumor activity of the diet. Various parameters are adjusted to maximize effectiveness of the treatment, including length of time the diet is administerred, cyclic vs. chronic methionine restriction, diet and chemotherapy vs. diet alone vs. chemotherapy alone, and so forth. Once the parameters are optimized for the mouse model, the regimen will be administered to human patients. As with many therapeutics in medicine, adjustments may be made upon administration to human patients to maximize effectiveness of the therapy, and one of skill in the art is knowledgeable about such routine modifications.

### REFERENCES

The following references and others cited herein but not listed here, to the extent that they provide exemplary procedural or other details supplementary to those set forth herein, are specifically incorporated herein by reference.

### Patents

U.S. Pat. No. 5,208,039

U.S. Pat. No. 5,571,510

U.S. Pat. No. 5,658,895

U.S. Pat. No. 5,690,929

U.S. Pat. No. 5,817,695

U.S. Pat. No. 6,004,930

U.S. Pat. No. 6,017,962

### PUBLICATIONS

American Institute for Cancer Research. “Food, Nutrition and the Prevention of Cancer: A Global Perspective.” World Cancer Research Fund, American Institute for Cancer Research, 1997.

Aw T Y, Ookhtens M, Kaplowitz N: Inhibition of glutathione efflux from isolated rat hepatocytes by methionine. J Biol Chem 259: 9355-9358, 1984.

Aw T Y, Ookhtens M, Kaplowitz N: Mechanism of inhibition of glutathione efflux by methionine from isolated rat hepatocytes. Am J Physiol 251: G354-G361, 1986.

Awram P, Smit J: The Caulobacter crescentus paracrystalline S-layer protein is secrerted by an ABC transporter (type 1) system. J. Bacteriol. 180: 3062-3069, 1998.

Baylin S B, Herman J G, Graff J R, Vertino P M, Issa J P: Alterations in DNA methylation: a fundamental aspect of neoplasia. Adv. Cancer Res 72:141-96, 1998.

Beckman K B, Ames B N: Oxidative decay of DNA. J Biol Chem 272: 19633-19636, 1997.

Bewly J D, Black M (eds.), Seeds-Physiology of Development and Germination, pp. 17-33 (1994).

Bingle W H, Nomellini J F, Smit J: Secretion of the Caulobacter crescentus S-layer protein: Further localization of the C-terminal secretion sequence signal and its use for secretion of recombinant proteins. J. Bacteriol. 182: 3298-3301, 2000

Breillout F, Antoine E, Poupon MF: Methionine dependency of malignant tumors: a possible approach for therapy. J Natl Cancer Inst 82: 1628-1632, 1990.

Breillout F, Hadida F, Echinard-Garin P, Lascaux V, Poupon M F. Decreased rat rhabdomyosarcoma pulmonary metastases in response to a low methionine diet. Anticancer Res. 1987;7:861-867.

Breillout F, Poupon M F, Blanchard P, Lascaux V, Echinard-Garin P, Robert-Gero M: Association of SIBA treatment and a Met-depleted diet inhibits in vitro growth and in vivo metastatic spread of experimental tumor cell lines. Clin Exp Metastasis 6: 3-16, 1988.

Calvert P, Yao KS, Hamilton T C, O'Dwyer P J: Clinical studies of reversal of drug resistance based on glutathione. Chem Biol Interact 111-112: 213-224, 1998.

Carson, J. P., Kulik, G., and Weber, M. J. (1999) Cancer Res. 59, 1449-1453.

Casey R, Domoney C, Ellis N: Legume storage proteins and their genes. Oxford Surveys of Plant Molecular and Cell Biology 3:1-95, 1986.

Chan T A, Hermeking H, Lengauer C, Kinzler K W, Vogelstein B: 14-3-3Sigma is required to prevent mitotic catastrophe after DNA damage. Nature 401: 616-620, 1999.

Chen X, Carystinos G D, Batist G: Potential for selective modulation of glutathione in cancer chemotherapy. Chem Biol Interact 111-112: 263-275, 1998.

Chiang P K, Gordon R K, Tal J, Zeng G C, Doctor B P, Pardhasaradhi K, McCann, P P: S-Adenosylmethionine and methylation. FASEB J 10: 471-480, 1996.

Cross, D. R., Miller, B. J., and James, S. J. (1993) Cell Prolif. 26, 327-336.

Davey C, Pennings S, Allan J: CpG methylation remodels chromatin structure in vitro. J. Mol. Biol 267: 276-288, 1997.

Davies K J: Oxidative stress: the paradox of aerobic life. Biochem Soc Symp 61: 1-31, 1995.

Demopoulos H B: Effects of reducing the phenylalanine-tyrosine intake of patients with advanced malignant melanoma. Cancer 19: 657-664, 1966.

Djurhuus R, Svardal A M, Mansoor M A, Ueland P M: Modulation of glutathione content and the effect on methionine auxotrophy and cellular distribution of homocysteine and cysteine in mouse cell lines. Carcinogenesis 12: 241-247, 1991.

Drummond J C: A comparative study of tumor and normal tissue growth. Biochem J 11:825, 1917.

Du Vigneaud V, Chandler J P, Moyer A W, Keppel D M. The effect of choline on the ability of homocysteine to replace methionine in the diet. J. Biol. Chem. 1939;131:57-76.

Evans M: Purification of recombinant proteins expressed with polyhistidine tags in mammalian cells. Am. Biotech. Lab. July 1998: 16-17, 1998.

Fernandez-Checa J C, Maddatu T, Ookhtens M, Kaplowitz N: Inhibition of GSH efflux from rat liver by methionine: effects of GSH synthesis in cells and perfused organ. Am J Physiol 258: G967-G973, 1990.

Fiskerstrand T, Christensen B, Tysnes O B, Ueland P M, Refsum H: Development and reversion of methionine dependence in a human glioma cell line: relation to homocysteine remethylation and cobalamin status. Cancer Res 54: 4899-4906, 1994.

Fox M E, Lemmon M J, Mauchline M L, Davis T O, Giaccia A J, Minton N P, Brown J M. Anaerobic bacteria as a delivery system for cancer gene therapy: in vitro activation of 5-fluorocytosine by genetically engineered clostridia. Gene Ther. February 1996; 3(2): 173-8.

Fu YM, Yu ZX, Pelayo BA, Ferrans VJ, Meadows GG: Focal adhesion kinase-dependent apoptosis of melanoma induced by tyrosine and phenylalanine deficiency. Cancer Res. 59: 758-765, 1999.

Gatehouse J A, Evans I M, Bown D, Croy R R D, Boulter D: Control of storage-protein synthesis during seed development in pea (Pisum sativum L.) Biochem. J. 208:119-127, 1982

Goldenberg D M. New developments in monoclonal antibodies for cancer detection and therapy. CA Cancer J. Clin. January-February 1994; 44(1):43-64.

Goseki N, Endo M, Onodera T, Kosaki G: Anti-tumor effect of L-methionine-deprived total parenteral nutrition with 5-fluorouracil administration on Yoshida sarcoma-bearing rats. Ann Surg 214: 83-88, 1991.

Goseki N, Endo M: Thiol depletion and chemosensitization on nimustine hydrochloride by methionine-depleting total parenteral nutrition. Tohoku J Exp Med 161: 227-239, 1990.

Goseki N, Nagahama T, Maruyama M, Endo M: Enhanced anticancer effect of vincristine with methionine infusion after methionine-depleting total parenteral nutrition in tumor-bearing rats. Jpn J Cancer Res 87: 194-199, 1996.

Goseki N, Onodera T, Koike M, Kosaki G: Inhibitory effect of L-methionine-deprived amino acid imbalance using total parenteral nutrition on growth of ascites hepatoma in rats. Tohoku J Exp Med 151: 191-200, 1987.

Goseki N, Yamazaki S, Endo M, Onodera T, Kosaki G, Hibino Y, Kuwahata T: Antitumor effect of methionine-depleting total parenteral nutrition with doxorubicin administration on Yoshida sarcoma-bearing rats. Cancer 69: 1865-1872, 1992.

Goseki N, Yamazaki S, Shimojyu K, et al. Synergistic effect of methionine-depleting total parenteral nutrition with 5-fluorouracil on human gastric cancer: a randomized, prospective clinical trial. Jpn. J. Cancer Res. 1995;86:484-489.

Goseki N, Yamazaki S, Shimojyu K, Kando F, Maruyama M, Endo M, Koike M, Takahashi H: Synergistic effect of methionine-depleting total parenteral nutrition with 5-fluorouracil on human gastric cancer: a randomized, prospective clinical trial. Jpn J Cancer Res 86: 484-489, 1995.

Greenberg N M, DeMayo F, Finegold M J, Medina D, Tilley W D, Aspinall J O, Cunha G R, Donjacour A A, Matusik R J, Rosen J M. Prostate cancer in a transgenic mouse. Proc Natl Acad Sci USA. Apr. 11, 1995;92(8):3439-43.

Guo H, Lishko V K, Herrera H, Groce A, Kubota T, Hoffman RM. Therapeutic tumor-specific cell cycle block induced by methionine starvation in vivo. Cancer Research 1993;53 :5676-5679.

Guo H, LishkoVK, Herrera H, Groce A, Kubota T, Hoffman, R M: Therapeutic tumor-specific cell cycle block induced by methionine starvation in vivo. Cancer Research 53: 5676-5679, 1993.

Guo H, Tan Y, Kubota T, Moossa A R, Hoffman R M: Methionine depletion modulates the antitumor and antimetastatic efficacy of methionine. Anticancer Res 16: 2719-2723, 1996.

Guo H Y, Herrera H, Groce A, Hoffman RM. Expression of the biochemical defect of methionine dependence in fresh patient tumors in primary histoculture. Cancer Res. 1993;53 :2479-2483.

Guo H Y, Hoffman R M, Herrera H: Unchecked DNA synthesis and blocked cell division induced by methionine deprivation in a human prostate cancer cell line. In Vitro Cell Dev Biol Anim 29A: 359-361, 1993.

Haldar, S., Jena, N., and Croce, C. M. (1995) Proc. Natl. Acad. Sci. U.S.A. 92, 4507-Haldar, S., Basu, A., and Croce, C. M. (1997) Cancer Res. 57, 229-233

Halpern B C, Clark B R, Hardy D N, Halpern R M, Smith R A. The effect of replacement of methionine by homocystine on survival of malignant and normal adult mammalian cells in culture. Proc. Natl. Acad. Sci. U.S.A. 1974;71:1133-1136.

Hoffman R M, Erbe R W: High in vivo rates of methionine biosynthesis in transformed human and malignant rat cells auxotrophic for methionine. Proc Natl Acad Sci U.S.A. 73: 1523-1527, 1976.

Hoffman R M: Altered methionine metabolism and transmethylation in cancer. Anticancer Res 5: 1-30, 1985.

Hoffman R M: Altered methionine metabolism, DNA methylation and oncogene expression in carcinogenesis. A review and synthesis. Biochim Biophys Acta 738: 49-87, 1984.

Hoppe-Seyler F, Butz K. Peptide aptamers: powerful new tools for molecular medicine. J Mol Med. 2000;78(8):426-30.

Hoshiya Y, Guo H, KubotaT, Inada T, Asanuma F, Yamada Y, Koh J, Kitajima M, Hoffman RM: Human tumors are methionine dependent in vivo. Anticancer Res 15: 717-718, 1995.

Hoshiya Y, Kubota T, Inada T, Kitajima M, Hoffman RM: Methionine-depletion modulates the efficacy of 5-fluorouracil in human gastric cancer in nude mice. Anticancer Res 17: 4371-4375, 1997.

Hoshiya Y, Kubota T, Matsuzaki SW, Kitajima M, Hoffman RM: Methionine starvation modulates the efficacy of cisplatin on human breast cancer in nude mice. Anticancer Res 16: 3515-3517, 1996.

Johnston, P. G., Lenz, H. J., Leichman, C. G., Danenberg, K. D., Allegra, C. J., Danenberg, P. V., and Leichman, L. (1995) Cancer Res. 55, 1407-1412.

Judde J G, Ellis M, Frost P: Biochemical analysis of the role of transmethylation in the methionine dependence of tumor cells. Cancer Res 49: 4859-4865, 1989.

Kjeldahl, Anal. Chem. (1883) 22:366.

Koivunen E, Arap W, Rajotte D, Lahdenranta J, Pasqualini Identification of receptor ligands with phage display peptide libraries. J Nucl Med. 1999 May;40(5):883-8.

Kokkinakis D M, Hoffman R M, Frenkel E P, et al. Synergy between Methionine Stress and Chemotherapy in the Treatment of Brain Tumor Xenografts in Athymic Mice. Cancer Res. 2001;61:4017-4023.

Kokkinakis D M, Schold S C J, Hori H, Nobori T. Effect of long-term depletion of plasma methionine on the growth and survival of human brain tumor xenografts in athymic mice. Nutr. Cancer 1997;29:195-204.

Kokkinakis D M, Von W M, Vuong T H, Brent T P, Schold S C J: Regulation of O6-methylguanine-DNA methyltransferase by methionine in human tumour cells. Br J Cancer 75: 779-788, 1997.

Kreis W, Baker A, Ryan V, Bertasso A: Effect of nutritional and enzymatic methionine deprivation upon human normal and malignant cells in tissue culture. Cancer Res 40: 634-641, 1980.

Kreis W, Hession C. Biological effects of enzymatic deprivation of L-methionine in cell culture and an experimental tumor. Cancer Res. 1973;33:1866-1869.

Kreis W: Tumor therapy by deprivation of L-methionine: rationale and results. Cancer Treat Rep 63: 1069-1072, 1979.

Kutzbach, C. and Stokstad, E. L. (1967) Biochim. Biophys. Acta 139, 217-220.

Lawson D H, Stockton L H, Bleier J C, Acosta P B, Heymsfield S B, Nixon, D W: The effect of a phenylalanine and tyrosine restricted diet on elemental balance studies and plasma aminograms of patients with disseminated malignant melanoma. Am J Clin Nutr41: 73-84, 1985.

Lemmon M J, van Zij I P, Fox M E, Mauchline M L, Giaccia A J, Minton N P, Brown J M. Anaerobic bacteria as a gene delivery system that is controlled by the tumor microenvironment. Gene Ther. August 1997;4(8):791-6.

Lorincz A B, Kuttner R E, Brandt M B: Tumor response to phenylalanine-tyrosine-limited diets. J Am Diet Assoc 54: 198-205, 1969.

Lu S, Epner D E: Molecular mechanisms of cell cycle block by methionine restriction in human prostate cancer cells. (2000) Nutr. Cancer 38, 123-130.

Magera M J, Lacey J M, Casetta B, Rinaldo P. Method for the determination of total homocysteine in plasma and urine by stable isotope dilution and electrospray tandem mass spectrometry. Clin. Chem. 1999;45:1517-1522.

Malley A, Baecher L, Mackler B, Perlman F: The isolation of allergens from the green pea. J. Allergy Clin. Immunol. 56:282-290, 1975.

Mecham J O, Rowitch D, Wallace C D, Stem P H, Hoffman R M. The metabolic defect of methionine dependence occurs frequently in human tumor cell lines. Biochem. Biophys. Res. Commun. 1983; 117:429-434.

Meredith M J, Cusick C L, Soltaninassab S, Sekhar K S, Lu S, Freeman M L: Expression of Bcl-2 increases intracellular glutathione by inhibiting methionine-dependent GSH efflux. Biochem Biophys Res Commun 248: 458-463, 1998.

Miki K, Al-Refaie W, Xu M, Jiang P, Tan, Y, Bouvet M, Zhao M, Gupta A, Chishima T, Shimada H, Makuuchi M, Moossa A R, Hoffman R M: Methioninase gene therapy of human cancer cells is synergistic with recombinant methioninase treatment. Cancer Res 60: 2696-2702, 2000.

Miki K, Xu M, An Z, Wang X, Yang M, Al-Refaie W, Sun X, Baranov E, TanY, Chishima T, Shimada H, Moossa A R, Hoffman R M: Survival efficacy of the combination of the methioninase gene and methioninase in a lung cancer orthotopic model. Cancer Gene Ther 7: 332-338, 2000.

Millis R M, Diya C A, Reynolds M E, Dehkordi O, Bond V J: Growth inhibition of subcutaneously transplanted hepatomas without cachexia by alteration of the dietary arginine-methionine balance. Nutr Cancer 31: 49-55, 1998.

Millis R M, Diya C A, Reynolds ME: Growth inhibition of subcutaneously transplanted hepatomas by alterations of the dietary arginine-methionine balance. Nutr Cancer 25: 317-327, 1996.

Monzavi-Karbassi B, Kieber-Emmons T. Current concepts in cancer vaccine strategies. Biotechniques. January 2001;30(1):170-2, 174, 176 passim. Myers JN. The use of biological therapy in cancer of the head and neck. Curr Probl Cancer. May-June 1999;23(3): 106-34.

Nagahama T, Goseki N, Endo M: Doxorubicin and vincristine with methionine depletion contributed to survival in the Yoshida sarcoma bearing rats. Anticancer Res 18: 25-31, 1998.

Nan X, Campoy F J, Bird A: MeCP2 is a transcriptional repressor with abundant binding sites in genomic chromatin. Cell 88: 471-481, 1997.

Niu C, Yan H, Yu T, Sun H P, Liu J X, Li X S, Wu W, Zhang F Q, Chen Y, Zhou L, Li J M, Zeng X Y, Yang R R, Yuan M M, Ren M Y, Gu F Y, Cao Q, Gu B W, Su X Y, Chen G Q, Xiong S M, Zhang T, Waxman S, Wang Z Y, Chen S J: Studies on treatment of acute promyelocytic leukemia with arsenic trioxide: remission induction, follow-up, and molecular monitoring in 11 newly diagnosed and 47 relapsed acute promyelocytic leukemia patients. Blood 94: 3315-3324, 1999.

Osborne T B, Campbell G F: Proteids of the Pea. J. Am. Chem. Soc. 20: 348-362, 1898.

Parr, A. L., Drake, J. C., Gress, R. E., Schwartz, G., Steinberg, S. M., and Allegra, C. J. (1998) Biochem. Pharmacol. 56, 231-235.

Pelayo B A, Fu Y M, Meadows G G: Inhibition of B16BL6 melanoma invasion by tyrosine and phenylalanine deprivation is associated with decreased secretion of plasminogen activators and increased plasminogen activator inhibitors. Clin. Exp. Metastasis 17: 841-848, 1999.

Pogribny I P, Basnakian A G, Miller B J, Lopatina N G, Poirier L A, James S J: Breaks in genomic DNA and within the p53 gene are associated with hypomethylation in livers of folate/methyl-deficient rats. Cancer Res 55: 1894-1901, 1995.

Poirson-Bichat F, Goncalves R A, Miccoli L, Dutrillaux B, Poupon M F: Methionine depletion enhances the antitumoral efficacy of cytotoxic agents in drug-resistant human tumor xenografts. Clin Cancer Res 6: 643-653, 2000.

Poirson-Bichat F, Gonfalone G, Bras-Goncalves R A, Dutrillaux B, Poupon M F. Growth of methionine-dependent human prostate cancer (PC-3) is inhibited by ethionine combined with methionine starvation. Br. J. Cancer 1997;75:1605-1612.

Poirson-Bichat F, Lopez R, Bras G R, Miccoli L, Bourgeois Y, Demerseman P, Poisson M, Dutrillaux B, Poupon M F: Methionine deprivation and methionine analogs inhibit cell proliferation and growth of human xenografted gliomas. Life Sci 60: 919-931, 1997.

Polyamines in Cancer: Basic Mechanisms and Clinical Approaches. Austin, Tex.: R. G. Landes Company, 1996.

Porter C W, Sufrin J R, Keith D D: Growth inhibition by methionine analog inhibitors of S-adenosylmethionine biosynthesis in the absence of polyamine depletion. Biochem Biophys Res Commun 122: 350-357, 1984.

Porter C W, Sufrin J R: Interference with polyamine biosynthesis and/or function by analogs of polyamines or methionine as a potential anticancer chemotherapeutic strategy. Anticancer Res 6: 525-542, 1986.

Raguso C A, Pereira P, Young V R. A tracer investigation of obligatory oxidative amino acid losses in healthy, young adults. Am. J. Clin. Nutr. 1999;70:474-483.

Razin A: CpG methylation, chromatin structure and gene silencing-a three-way connection. EMBO J. 17: 4905-4908, 1998.

Reed, J. C. (1998) Oncogene 17, 3225-3236

Remington's Pharmaceutical Sciences, 18th Ed. Mack Printing Company, 1990.

Rodriguez-Vicente J, Vicente-Ortega V, Canteras-Jordana M: The effects of different antineoplastic agents and of pretreatment by modulators on three melanoma lines. Cancer 82: 495-502, 1998.

Rose W C. Amino acid requirements of man. Federation Proceedings 1949;8:546-552.

Salunke D K, Jadhav, S J, Kadam S S, Chavan J K: Chemical, biochemical, and biological significance of polyphenols in cereals and legumes. Critical Reviews in Food Science and Nutrition 17:277-305, 1982.

Santini V, Kantarjian H M, Issa J P. Changes in DNA methylation in neoplasia: pathophysiology and therapeutic implications. Ann. Intern. Med. 2001; 134:573-586.

Sasamura T, Matsuda A, Kokuba Y: Effects of D-methionine-containing solution on tumor cell growth in vitro. Arzneimittelforschung 49: 541-543, 1999.

Sasamura T, Matsuda A, Kokuba Y: Nutritional effects of a D-methionine-containing solution on AH109A hepatoma-bearing rats. Biosci Biotechnol Biochem 62: 2418-2420, 1998.

Schroeder H E: Quantitative studies on the cotyledonary proteins in the genus Pisum. J. Sci. Food Agric. 33:623-633, 1982.

Scott, J. M. and Weir, D. G. (1981) Lancet 2, 337-340.

Stem P H, Hoffman R M. Enhanced in vitro selective toxicity of chemotherapeutic agents for human cancer cells based on a metabolic defect. J. Natl. Cancer Inst. 1986;76:629-639.

Stem P H, Hoffman R M: Elevated overall rates of transmethylation in cell lines from diverse human tumors. In Vitro 20: 663-670, 1984.

Stem P H, Hoffman R M: Enhanced in vitro selective toxicity of chemotherapeutic agents for human cancer cells based on a metabolic defect. J Natl Cancer Inst 76: 629-639, 1986.

Stem P H, Wallace C D, Hoffman R M: Altered methionine metabolism occurs in all members of a set of diverse human tumor cell lines. J Cell Physiol 119: 29-34, 1984.

Story M D, Meyn R E: Modulation of apoptosis and enhancement of chemosensitivity by decreasing cellular thiols in a mouse B-cell lymphoma cell line that overexpresses bcl-2. Cancer Chemother Pharmacol 44: 362-366, 1999.

Suganuma M, Kawabe T, Hori H, Funabiki T, Okamoto T: Sensitization of cancer cells to DNA damage-induced cell death by specific cell cycle G2 checkpoint abrogation. Cancer Res 59: 5887-5891, 1999.

Sugimura T, Birnbaum S M, Winitz M, Greenstein J P: Quantitative nutritional studies with water-soluble, chemically defined diets. VIII. The forced feeding of diets each lacking in one essential amino acid. Arch Biochem Biophys 81: 448-455, 1959.

Tan Y, Sun X, Xu M, An Z, Tan X, Han Q, Miljkovic D A, Yang M, Hoffman R M: Polyethylene glycol conjugation of recombinant methioninase for cancer therapy. Protein Expr Purif 12: 45-52, 1998.

Tan Y, Sun X, Xu M, Tan X, Sasson A, Rashidi B, Han Q, Wang X, An Z, Sun F X, Hoffman R M: Efficacy of recombinant methioninase in combination with cisplatin on human colon tumors in nude mice. Clin Cancer Res 5: 2157-2163, 1999.

Tan Y, Xu M, Guo H, Sun X, Kubota T, Hoffman R M: Anticancer efficacy of methioninase in vivo. Anticancer Res 16: 3931-3936, 1996.

Tan Y, Zavala J S, Han Q, Xu M, Sun X, Tan X, Magana R, Geller J, Hoffman R M: Recombinant methioninase infusion reduces the biochemical endpoint of serum methionine with minimal toxicity in high-stage cancer patients. Anticancer Res 17: 3857-3860, 1997.

Tan Y, Zavala J S, Xu M, Zavala J J, Hoffman R M: Serum methionine depletion without side effects by methioninase in metastatic breast cancer patients. Anticancer Res 16: 3937-3942, 1996.

Tang, D., Lahti, J. M., and Kidd, V. J. (2000) J. Biol. Chem. 275, 9303-9307

Tisdale M, Eridani S. Methionine requirement of normal and leukaemic haemopoietic cells in short term cultures. Leuk. Res. 1981;5:385-394.

Tisdale M, Eridani S: Methionine requirement of normal and leukaemic haemopoietic cells in short term cultures. Leuk Res 5: 385-394, 1981.

Tisdale M J, Jack G W, Eridani S: Differential sensitivity of normal and leukaemic haemopoietic cells to methionine deprivation by L-methioninase. Leuk Res 7: 269-277, 1983.

Tisdale M J. Utilization of preformed and endogenously synthesized methionine by cells in tissue culture. Br. J. Cancer 1984;49:315-320.

Tisdale M J: Changes in tRNA methyltransferase activity and cellular S-adenosylmethionine content following methionine deprivation. Biochim Biophys Acta 609: 296-305, 1980.

Tisdale M J: Effect of methionine deprivation on methylation and synthesis of macromolecules. Br J Cancer 42: 121-128, 1980.

Tournier, C., Hess, P., Yang, D. D., Xu, J., Turner, T. K., Nimnual, A., Bar-Sagi, D., Jones, S. N., Flavell, R. A., and Davis, R. J. (2000) Science 288, 870-874

Van der Wilt, C. L., Pinedo, H. M., Smid, K., and Peters, G. J. (1992) Cancer Res. 52, 4922-4928.

Wang S T, Chen H W, Sheen L Y, Lii C K: Methionine and cysteine affect glutathione level, glutathione-related enzyme activities and the expression of glutathione S-transferase isozymes in rat hepatocytes. J Nutr 127: 2135-2141, 1997.

Weiner L M. Monoclonal antibody therapy of cancer. Semin Oncol. October 1999;26(5 Suppl 14):43-51.

Wolffe A: Chromatin Structure. In: “Chromatin: Structure and Function”, San Diego: Academic Press. pp 7-172, 1998.

Yang T-T, Nelson P S, Bush G L, Meyer D I, Kain S R: An immobilized Co2+ matrix for purification of 6xhistidine tagged proteins. Am. Biotech. Lab. January 1997, 12-14, 1997.

Yazawa K, Fujimori M, Amano J, Kano Y, Taniguchi S. Bifidobacterium longum as a delivery system for cancer gene therapy: selective localization and growth in hypoxic tumors. Cancer Gene Ther. 2000 Feb;7(2):269-74.

Yoshida S, Yamana H, Tanaka T, Ishibashi N, Toh U, Ishii H, Shirouzu Y, Shirouzu K: Effect of combination therapy with a methionine-mitomycin C conjugate and a methionine-deficient diet on tumor growth. In Vivo 12: 351-355, 1998.

Yoshioka T, Wada T, Uchida N, et al. Anticancer efficacy in vivo and in vitro, synergy with 5-fluorouracil, and safety of recombinant methioninase. Cancer Res. 1998;58:2583-2587.

Young V R, el-Khoury A E. Can amino acid requirements for nutritional maintenance in adult humans be approximated from the amino acid composition of body mixed proteins? Proc. Natl. Acad. Sci. U.S.A 1995;92:300-304.

One skilled in the art readily appreciates that the patent invention is well adapted to carry out the objectives and obtain the ends and advantages mentioned as well as those inherent therein. Methods, procedures, techniques, and kits described herein are presently representative of the preferred embodiments and are intended to be exemplary and are not intended as limitations of the scope. Changes therein and other uses will occur to those skilled in the art which are encompassed within the spirit of the invention or defined by the scope of the pending claims.

We claim:

1. A method of inducing apoptosis in a mammalian cancer cell, comprising the steps of:
inducing cell-cycle arrest by methionine depletion in the cell; and
abrogating the arrest by methionine repletion in the cell.

2. The method of claim 1, wherein the inducing and abrogating steps are repeated at least once.

3. A method of treating cancer in a mammal, comprising the steps of:
a) delivering to the mammal at least one methionine level-reducing agent for a sufficient time to induce cell cycle arrest in a cancer cell of said mammal; and
b) replenishing said methionine, wherein said replenishment results in induction of apoptosis in said cancer cell.

4. The method of claim 3, wherein said delivering and replenishing steps are repeated at least once.

5. The method of claim 3, wherein said methionine level-reducing agent is a methionine-restricted diet.

6. The method of claim 5, wherein said methionine-restricted diet comprises methionine levels no greater than about 2 mg/kg/day.

7. The method of claim 5, wherein the methionine restricted diet comprises an intact protein having less than about 0.2% (w/w) methionine.

8. The method of claim 7, wherein the intact protein is from a legume.

9. The method of claim 8, wherein the legume is broad bean or garden pea.

10. The method of claim 3, wherein the methionine level-reducing agent is an enzyme.

11. The method of claim 10, wherein the enzyme is methioninase.

12. The method of claim 3, wherein the delivery of the methionine level-reducing agent further comprises the steps of:
a) administering to the mammal a diet substantially lacking in methionine for a time t₁; and
b) administering to the mammal a methionine-restricted diet for a time t₂.

13. The method of claim 12, wherein the time t₁ is at least approximately 1 week.

14. The method of claim 12, wherein the time t₂ is at least approximately 1 week.

15. The method of claim 12, wherein the combination of the time t₁ and the time for t₂ is between about 3 weeks and about 15 weeks.

16. The method of claim 3, wherein the replenishing step further comprises administering to the mammal a methionine-replete diet for a time t₃.

17. The method of claim 16, wherein the time t₃ is about 1 week.

18. A method of treating cancer in a mammal, comprising the steps of:
a) administering to the mammal a diet substantially lacking in methionine for a time t₁;
b) administering to the mammal a methionine-restricted diet for a time t₂; and
c) administering to the mammal a diet replete with methionine for a time t₃.

19. The method of claim 18, wherein said methionine-restricted diet comprises methionine levels no greater than about 2 mg/kg/day.

20. The method of claim 18, wherein the methionine restricted diet comprises an intact protein having less than about 0.2% (w/w) methionine.

21. The method of claim 20, wherein the intact protein is from a legume.

22. The method of claim 21, wherein the legume is broad bean or garden pea.

23. The method of claim 18, wherein the time t₁ is at least approximately 1 week.

24. The method of claim 18, wherein the time t₂ is at least approximately 1 week.

25. The method of claim 18, wherein the combination of the time t, and the time t₂ is between about 3 weeks and about 15 weeks.

26. The method of claim 18, wherein the time t₃ is about 1 week.

27. The method of claim 18, wherein at least one step in the method comprises enteral administration of the diet to the individual.

28. The method of claim 18, wherein said cancer is prostate, lung, breast, colon, glioma, gastric, skin, esophagus, squamous cell carcinoma of head and neck region, pancreas, small intestine, bladder and urinary collecting system, kidney, testes, ovary, rectum, anus, liver, brain, soft tissue or osteogenic sarcoma, Hodgkin's lymphoma, non-Hodgkin's lymphoma, leukemia, or melanoma.

29. The method of claim 18, wherein said method is repeated at least once.

30. The method of claim 18, wherein said cancer is an advanced cancer.

31. The method of claim 18, further comprising the step of treating the cancer with chemotherapy, surgery, radiation, gene therapy, immunotherapy, biological therapy, differentiating agents, chemopreventive agents, or a combination thereof.

32. A method for improving efficacy of a cancer therapy for a mammal, comprising the steps of:
delivering to the mammal at least one methionine level-reducing agent for a time t₁; and
administering said cancer therapy.

33. The method of claim 32, wherein delivery of said methionine level reducing agent is prior to the administration of the cancer therapy.

34. The method of claim 32, wherein delivery of said methionine level reducing agent is during the administration of the cancer therapy.

35. The method of claim 32, wherein said methionine level-reducing agent is administered following the administration of the cancer therapy.

36. The method of claim 32, wherein said methionine level-reducing agent is a methionine restricted diet.

37. The method of claim 32, wherein said cancer therapy is chemotherapy.

38. The method of claim 32, wherein said cancer therapy is radiation.

39. The method of claim 32, wherein said cancer therapy is surgery.

40. The method of claim 32, wherein said cancer therapy is chemotherapy, radiation, surgery, or a combination thereof.

41. The method of claim 36, wherein said methionine-restricted diet comprises methionine levels no greater than about 2 mg/kg/day.

42. The method of claim 36, wherein the methionine restricted diet comprises an intact protein having less than about 0.2% (w/w) methionine.

43. The method of claim 42, wherein the intact protein is from a legume.

44. The method of claim 43, wherein the legume is broad bean or garden pea.

45. The method of claim 32, wherein the time t₁ is at least approximately 1 week.

46. A nutritional composition comprising an isolated intact protein having less than about 0.2% (w/w) methionine.

47. A nutritional composition for an individual with cancer, comprising an intact protein having less than about 0.2% (w/w) methionine.

48. The composition of claim 47, wherein said intact protein is from a legume.

49. The composition of claim 48, wherein said legume is garden pea or broad bean.

50. A nutritional composition for an individual with cancer, wherein said composition comprises:
a protein system comprising:
from zero to about 0.3% (w/w) methionine; and
no more than about 1.5% (w/w) cyst(e)ine.

51. The composition of claim 50, wherein said composition provides about 15-30% of calories from protein.

52. The nutritional composition of claim 50, wherein said composition substantially lacks homocysteine.

53. The nutritional composition of claim 50, wherein said composition further comprises choline, cobalamine, and folate.

54. A method of treating cancer in a human, consisting essentially of administering to the human a methionine-restricted diet.

55. A method of treating cancer in a human, consisting essentially of administering to the human a diet substantially lacking methionine.

56. The method of claim 54, further comprising administering to said human a cancer therapy.

57. The method of claim 55, further comprising administering to said human a cancer therapy.
